(19) United States
(12) Patent Application Publication
(10) Pub. No.: US 2025/0049887 A1
(43) Pub. Date: Feb. 13, 2025

(54) METHODS AND COMPOSITIONS FOR MODULATING THE MYELOID ARM OF THE IMMUNE SYSTEM

(71) Applicant: Fred Hutchinson Cancer Center, Seattle, WA (US)

(72) Inventors: Lev Silberstein, Seattle, WA (US); Hans-Peter Kiem, Seattle, WA (US); Stefan Radtke, Seattle, WA (US)

(73) Assignee: Fred Hutchinson Cancer Center, Seattle, WA (US)

(21) Appl. No.: 18/717,971

(22) PCT Filed: Dec. 9, 2022
(86) PCT No.: PCT/US2022/081317

§ 371 Date: Jun. 7, 2024

Related U.S. Application Data
(60) Provisional application No. 63/287,934, filed on Dec. 9, 2021.

(51) Int. Cl. A61K 38/17

(52) U.S. Cl. CPC A61K 38/1774

(57) ABSTRACT

Methods and compositions for modulating the myeloid arm of the immune system are described. The methods and compositions modulate the myeloid arm of the immune system by stimulating or inhibiting Sema4 and/or a PlexinD1 receptor. Stimulating Sema4 and/or a PlexinD1 receptor down-regulates or dampens the myeloid arm of the immune system while inhibiting Sema4 and/or a PlexinD1 receptor up-regulates or heightens the myeloid arm of the immune system. The ability to modulate the myeloid arm of the immune system provides many advances such as reducing immune-responsiveness to various forms of stress and enhancing immune responsiveness to physiological challenges.

### CROSS REFERENCE TO RELATED APPLICATION

This application is a U.S. National Phase based on International Patent Application No. PCT/US2022/081317, filed on Dec. 9, 2022, which claims priority to U.S. Provisional Patent Application No. 63/287,934 filed Dec. 9, 2021, the contents of both of which are incorporated by reference herein in their entirety.

### FIELD OF THE DISCLOSURE

The current disclosure provides methods and compositions for modulating the myeloid arm of the immune system. The methods and compositions modulate the myeloid arm of the immune system by stimulating or inhibiting Sema4 and/or a PlexinD1 receptor. Stimulating Sema4 and/or a PlexinD1 receptor down-regulates or dampens the myeloid arm of the immune system while inhibiting Sema4 and/or a PlexinD1 receptor up-regulates or heightens the myeloid arm of the immune system. The ability to modulate the myeloid arm of the immune system provides many advances, such as in reducing immune-responsiveness to various forms of stress.

### BACKGROUND OF THE DISCLOSURE

Stem cells are unspecialized cells that are able to renew themselves through cell division for long periods. Stem cells can be subdivided and classified on the basis of their potency. A totipotent stem cell is produced from fusion between an egg and a sperm. Stem cells produced by the first few divisions of the fertilized egg cell are also totipotent stem cells. These cells can grow into any type of cell that makes up the developing organism, extra-embryonic tissues (e.g., yolk sac and placenta) and embryo. Pluripotent stem cells are descendants of totipotent cells that can differentiate into any cell type except for placental cells. Multipotent stem cells are descendants of pluripotent stem cells that can produce only cells of a closely related family of cells.

Hematopoietic stem cells (HSC) are pluripotent stem cells that ultimately give rise to all types of terminally differentiated blood cells. HSC can self-renew, or can differentiate into more committed progenitor cells, which progenitor cells are irreversibly determined to be ancestors of only a few types of blood cell. For instance, HSC can differentiate into (i) myeloid progenitor cells, which myeloid progenitor cells ultimately give rise to monocytes and macrophages, neutrophils, basophils, eosinophils, erythrocytes, megakaryocytes/platelets, dendritic cells, or (ii) lymphoid progenitor cells, which lymphoid progenitor cells ultimately give rise to T-cells, B-cells, and lymphocyte-like cells called natural killer cells (NK-cells). Once the stem cell differentiates into a myeloid progenitor cell, its progeny cannot give rise to cells of the lymphoid lineage, and, similarly, lymphoid progenitor cells cannot give rise to cells of the myeloid lineage.

HSC can also be classified as myeloid-biased HSC (myHSC) or balanced HSC (balHSC). myHSC preferentially give rise to myeloid lineage cells through myeloid committed progenitors while balHSC make substantially equal contributions to both myeloid and lymphoid lineages.

### SUMMARY OF THE DISCLOSURE

The current disclosure provides methods and compositions for modulating the myeloid arm of the immune system. The methods and compositions modulate the myeloid arm of the immune system by stimulating or inhibiting Sema4 and/or a PlexinD1 receptor. Stimulating Sema4 and/or a PlexinD1 receptor down-regulates or dampens the myeloid arm of the immune system while inhibiting Sema4 and/or a PlexinD1 receptor up-regulates or heightens the myeloid arm of the immune system. The ability to modulate the myeloid arm of the immune system provides many advances, such as reducing immune-responsiveness to various forms of stress.

In certain examples, disclosed methods and compositions maintain myeloid-biased hematopoietic stem cells (myHSC) in the presence of inflammatory insults or induce proliferation of myHSC in response to a physiological challenge. Additional uses are described in more detail elsewhere herein.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIGS. 1A-1G. (1A) Expression of Sema4a in single proximal and distal osteolineage cells (OLCs). The joint posteriors (black lines) describe the overall estimation of likely expression levels within the proximal (top) and distal (bottom) OLCs and are used to estimate the posterior of the expression fold difference (middle plot). The shaded area under the fold-difference posterior shows 95% confidence region. FPM, fragments per million. (1B) Ex vivo expansion of mouse lin−kit+Sca1+ (LKS) cells 24 hours after addition of mouse Sema4a-Fc/lgG1 control protein (n=5). (1C) Ex vivo proliferation of human CD34+ cells 24 hours after addition of human Sema4a-Fc/lgG1 protein (n=5). Estimated number of divided cells and representative carboxyfluorescein succinimidyl ester (CSFE) fluorescence histograms are shown. (1D) Immunophenotypic analysis of the bone marrow from young wild type (WT) and Sema4a knockout (Sema4aKO) mice (n=3-6). (1E) Hematopoietic stem cell (HSC) cell cycle analysis using DAPI/Ki-67 (4′,6-diamidino-2-phenylindole/Ki-67) staining in young WT and Sema4aKO mice (n=6). (1F) Gene set enrichment analysis (GSEA) of single cell RNA-Seq data for the HSC cluster (FIG. 2K) from young WT and Sema4aKO mice. False discover rate (FDR)<0.01, top five enriched pathways are shown. (1G) GSEA plots for the pathways that were enriched in young Sema4aKO HSC.

FIGS. 2A-2L. (2A) Sema4a mRNA expression in mouse niche cell subsets by bulk RNA-Seq (n=3). (2B) Sema4a mRNA expression in mouse niche cells by single cell RNA-Seq based on the data by Baccin et al. (2C) Sema4a mRNA expression in niche cell subsets from young vs aged WT mice (n=3). (2D) Human Sema4a mRNA expression in bone-lining cells (left panel) and endothelium (right panel) by single-molecule RNA fluorescence in situ hybridization (FISH) (RNAScope®, Advanced Cell Diagnostics, Inc., Newark, CA). (2E) Number of hematopoietic colonies (colony forming units, CFU) in the presence of mouse Sema4a-Fc/lgG1 control protein (n=6). (2F) Example of inhibitory effect of human Sema4a-Fc protein on ex vivo proliferation of human CD34+ cells (Donor 2) (n=3). (2G) Complete blood count data from young WT/Sema4a KO mice (n=6). (2H) Gating strategy for flow cytometric analysis of hematopoietic stem and progenitor cells (HSPCs) and isolation of myeloid-biased HSC (myHSC) and “balanced” HSC (balHSC) by cell sorting. (2I) Representative flow cytometry plots for cell cycle analysis of HSC from young WT/Sema4a mice. (2J) Short-term 5-ethynyl-2′-deoxyuridine (EdU) incorporation (cumulative data and representative plots) by HSC from WT and Sema4aKO mice (n=5). (2K, 2L): Uniform Manifold Approximation and Projection (UMAP) of 13417 WT (n=1) and Sema4aKO (n=2) lin-kit+ bone marrow cells with Leiden clustering. Distribution of WT and Sema4aKO cells within UMAP are shown. Cluster 1, highlighted with an asterisk, is determined to be HSC due to expression of HSC markers Ly6a and Hlf shown in FIG. 2L, a mean expression dotplot of 3 markers per cluster. Data are presented as mean±standard deviation (SD) *p<0.05; **p<0.01; ***p<0.001.

FIGS. 3A-3L. (3A) Experimental schema for the acute inflammatory stress model. (3B) Cell cycle analysis of myHSC 24 hours after injection with polyinosinic:polycytidilic acid (Poly(I:C) (n=5). (3C) Cell cycle analysis of balHSC 24 hours after injection with Poly(I:C) (n=5). (3D) WT vs Sema4a KO GSEA of myHSC from Poly(I:C) injected mice, FDR<0.01. (3E) GSEA plots and top differentially expressed genes for the pathways that were enriched in Sema4aKO myHSC after Poly(I:C) injection. (3F) Experimental schema for the transplant studies shown in (FIG. 3F) and (FIG. 3G). (3G) Donor chimerism in the recipients of myHSC from young WT and Sema4aKO mice (n=5). (3H) Donor chimerism in the recipients of balHSC from young WT and Sema4aKO mice (n=4). (3I) Published HSC gene expression data (Cabezas-Wallscheid et al., 2014, Cell Stem Cell, 15, 507-522) showing expression of known Sema4a receptors. (3J) Representative histograms (left panel) and quantification of mean fluorescence intensity of green fluorescent protein (GFP) expression (right panel) in myHSC and baIHSC from PlexinD1 (PlxnD1)-GFP reporter mice (n=3). In the left figure, CD150 high represent myHSC and CD150 low represents baIHSC. (3K) Donor chimerism in the recipients of myHSC from PlxnD1^{fl/fl}Cre⁺ and PlxnD1^{fl/fl}Cre⁻ mice (n=5). (3L) Donor chimerism in the recipients of baIHSC from PlxnD1^{fl/fl}Cre⁺ and PlxnD1^{fl/fl}Cre⁻ mice (n=5). Data are presented as mean±SD *p<0.05; **p<0.01; ***p<0.001.

FIGS. 4A-4Q. (4A) Gating strategy for Poly (1:C) injection experiments. (4B) Representative myHSC cell cycle plots from Poly (1:C)-injected WT and Sema4aKO mice (n=5). (4C) Representative baIHSC cell cycle plots from Poly (1:C)-injected mice WT and Sema4aKO mice (n=5). (4D) Bar graph and representative myHSC cell cycle plots from phosphate buffered saline (PBS)-injected WT and Sema4aKO mice (n=3). (4E) Bar graph and representative baIHSC cell cycle plots from PBS-injected WT and Sema4aKO (n=3). (4F) GSEA of balHSC from WT and Sema4aKO Poly(I:C) injected mice, FDR<0.01. (4G) Gating strategy for post-transplant chimerism analysis. (4H) Lineage composition of donor-derived cells from WT mice transplanted with WT and Sema4aKO myHSC (n=5). (4I) Lineage composition of donor-derived cells from WT mice transplanted with WT and Sema4aKO balHSC (n=4). (4J) PlexinD1 expression in human CD34+CD90+ cells. (4K) PlxnD1 excision validation by PCR analysis of genomic DNA from LKS cells as per indicated genotypes. The ladder on the left is a 1kb Plus DNA Ladder. (4L) PlxnD1 quantitative polymerase chain reaction (Q-PCR) analysis of mRNA from LKS cells as per indicated genotypes. (4M) Immunophenotypic analysis of the bone marrow from PlxnD1^{fl/fl} Mx1Cre⁺ and PlxnD^{fl/fl} Mx1Cre⁻ mice (n=4-7). (4N) HSC cell cycle analysis in PlxnD1^{fl/fl} Mx1Cre⁺ and PlxnD^{fl/fl} Mx1Cre⁻ mice (n=4-7). (4O) Experimental schema for the transplant studies shown in FIG. 3K and FIG. 3L. (4P) Lineage composition of donor-derived cells in the recipients of myHSC from PlxnD1^{fl/fl} Mx1Cre⁺ and PlxnD^{fl/fl} Mx1Cre⁻ mice (n=5). (4Q) Lineage composition of donor-derived cells in the recipients of balHSC from PlxnD1^{fl/fl} Mx1Cre⁺ and PlxnD^{fl/fl} Mx1Cre⁻ mice (n=5). Data are presented as mean±SD *p<0.05; **p<0.01; ***p<0.001.

FIGS. 5A-5N. (5A) Peripheral blood counts of WT and Sema4aKO mice during aging (n=4-9). (5B) Immunophenotypic analysis of the bone marrow from aged WT and Sema4aKO mice (n=4/5). (5C) Frequency of myHSC and balHSC in aged WT and Sema4aKO mice (representative flow cytometry plots shown on the right) (n=4/5). (5D) Experimental schema for transplant studies shown in FIG. 5E and FIG. 5F. (5E) Donor chimerism in the recipients of myHSC from aged WT and Sema4aKO mice (n=4/5). (5F) Donor chimerism in the recipients of balHSC from aged WT and Sema4aKO mice (n=5). (5G) UMAP representation of 162 myHSC from aged WT and Sema4aKO mice. (5H) UMAP representation of 165 balHSC cells from WT and Sema4aKO mice. (5I) Distribution of pairwise Spearman's correlation distances between aged WT and Sema4aKOmyHSC (left) and balHSC (right). The two distributions are statistically significantly different according to a Wilcoxon rank-sum test (p-value=3.1e-85). (5J) GSEA of myHSC from aged WT and Sema4aKO mice, FDR<0.01. (5K) GSEA plots and top differentially expressed genes for pathways enriched in Sema4aKO myHSC. (5L) Percentage of myHSC and balHSC cells from aged WT and Sema4aKO in the G2M phase of the cell cycle as estimated from their transcriptome using Cyclone. (5M, 5N) Distributions of diffusion pseudotime values of myHSC (5M) and balHSC (5N) from aged WT/Sema4aKO. The P-values shown at the bottom were computed with a Wilcoxon-rank sum test. Data are presented as mean±SD *p<0.05; **p<0.01; ***p<0.001.

FIGS. 6A-6O. (6A) Plasma level of pro-inflammatory cytokines in aged WT and Sema4aKO mice (n=5). (6B) Absolute number of HSPC and mature cells from aged WT and Sema4aKO mice (n=4). (6C) Absolute number of myHSC and balHSC in aged WT and Sema4aKO mice (n=4). (6D) Lineage composition of donor-derived cells in the recipients of myHSC from aged WT and Sema4aKO mice (n=4/5). (6E) Lineage composition of donor-derived cells in the recipients of balHSC from aged WT and Sema4aKO mice (n=5). (6F) Scaled mean expression of HSC cell cycle/self-renewal genes in WT balHSC vs WT myHSC from aged mice. (6G) Single cell expression (and corresponding FDR values) of HSC cell cycle/self-renewal genes which were significantly downregulated in aged Sema4aKO myHSC. (6H) GSEA plot and top differentially expressed genes for the “core aging signature” in aged WT and Sema4aKO myHSC. (6I) GSEA of balHSC from aged WT and Sema4aKO mice, FDR<0.01. (6J) UMAP of 8531 WT (N=2) Kit+ bone marrow cells with Leiden clustering. (6K) Dotplots of mean expression for selected markers. HSC (cluster 2) and multipotent progenitors (MPP) (cluster 0) are highlighted. (6L) Direct lineage relationship between cluster 2 and cluster 4, as predicted by graph abstraction (PAGA). (6M) Diffusion map representing HSC differentiation trajectory. Cells are colored based on diffusion pseudotime (DPT) coordinates. (6N) Expression of HSC/MPP marker genes along the DPT trajectory. Cells are colored based on their normalized expression levels for each the gene indicated at the top. (6O) Distributions of diffusion pseudotime values of WT aged myHSC and balHSC. The P-value is estimated with a Wilcoxon-rank sum test. Data are presented as mean±SD *p<0.05; **p<0.01; ***p<0.001.

FIGS. 7A-7U. (7A) Sema4a excision validation by PCR analysis of genomic DNA from LKS cells as per indicated genotypes. (7B) Sema4a Q-PCR analysis of mRNA from LKS cells as per indicated genotypes. (7C) Immunophenotypic analysis of the bone marrow from Sema4a^{fl/fl} Mx1Cre⁺ and Sema4a^{fl/fl}′ Mx1Cre⁻ mice (n=4-6). (7D) HSC cell cycle analysis in Sema4a^{fl/fl} Mx1Cre⁺ and Sema4a^{fl/fl}′ Mx1Cre⁻ mice (n=4). (7E) Experimental schema for the transplantation experiment shown in FIGS. 7F and 7G. (7F) Lineage composition of donor-derived cells in the recipients of myHSC from Sema4a^{fl/fl} Mx1Cre⁺ and Sema4^{fl/fl} Mx1Cre⁻ mice (n=4-5). (7G) Lineage composition of donor-derived cells in the recipients of baIHSC from Sema4a^{fl/fl} Mx1Cre⁺ and Sema4a^{fl/fl} Mx1Cre⁻ mice (n=4-5). (7H, 7I) Post-transplant lymphocyte and platelet counts for WT and Sema4aKO recipients of myHSC (panel H) and baIHSC (panel I) (n=9-11). (7J) Immunophenotypic analysis of the bone marrow from Sema4a^{fl/fl} Osx Cre⁺ and Sema4a^{fl/fl} Osx Cre⁻ mice (n=4). (7K) HSC cell cycle analysis in Sema4a^{fl/fl} Osx Cre⁺ and Sema4a^{fl/fl} Osx Cre⁻ mice (n=4). (7L) Immunophenotypic analysis of the bone marrow from Sema4a^{fl/fl}′ vascular endothelial (VE)-cadherin Cre ERT2 (+) and Sema4a^{fl/fl} VE-cadherin Cre ERT2 (−) mice (n=5). (7M) HSC cell cycle analysis in Sema4a^{fl/fl} VE-cadherin Cre ERT2 (+) and Sema4a^{fl/fl} VE-cadherin Cre ERT2 (−) mice (n=5). (7N) Experimental schema for the transplantation experiment using Sema4a^{fl/fl} Osx Cre⁺ and Sema4a^{fl/fl} Osx Cre⁻ recipient mice. (7O, 7P) Survival curves from the experiments depicted in FIG. 7N for myHSC (7O) and balHSC (7P) (n=7-10 and n=7/8). (7Q) Experimental schema for the transplantation experiment using Sema4a^{fl/fl} VE-cadherin Cre ERT2 (+) and Sema4a^{fl/fl} VE-cadherin Cre ERT2 (−) recipient mice. (7R, 7S) Post-transplant blood counts from experiments depicted in FIG. 7R for myHSC (7R) and balHSC (7S) (n=7 and n=5-7). (7T) Representative images of single cells and clusters from intra-vital imaging of calvarial bone marrow. (7U) Number of clusters 18-24 hours after transplantation of WT myHSC or balHSC into WT and Sema4aKO recipients, as assessed by two-photon intravital imaging of the calvarial bone marrow (n=4-6). Data are presented as mean±SD *p<0.05; **p<0.01; ***p<0.001.

FIGS. 8A-8I. (8A) Donor chimerism in the recipients of myHSC from Sema4a^{fl/fl}Cre⁺ and Sema 4a^{fl/fl} Cre⁻ mice (n=4-5). (8B) Donor chimerism in the recipients of balHSC from Sema4a^{fl/fl}Cre⁺ and Sema 4a^{fl/fl} Cre⁻ mice (n=4-5). (8C) Experimental schema for non-competitive transplant experiments shown in FIGS. 8D and 8E. (8D) Survival curve and peripheral blood counts in WT and Sema4aKO recipients of myHSC (n=9-11). (8E) Survival curve and peripheral blood counts in WT and Sema4aKO recipients of balHSC (n=9-11). (8F) Number of total cells 18-24 hours after transplantation of WT myHSC or balHSC into WT and Sema4aKO recipients, as assessed by two-photon intravital imaging of the calvarial bone marrow (n=4-6). (8G) Quantification of 3D distances between individual transplanted cells and the nearest endosteal surface (n=85, 331, 72, and 122 cells per group for WT myHSCs, Sema4aKO myHSCs, WT balHSCs, and Sema4aKO balHSCs, respectively). (8H, 8I) Cell displacement over time determined by two-photon time-lapse microscopy for WT myHSCs transplanted into WT (8H) and Sema4aKO (8I) recipients, respectively. These 4 myHSCs in Sema4aKO recipients were the only cells found to be motile over all imaging experiments for all mice. Due to high motility, all 4 of these cells migrated out of the field of view and were not tracked over the full 1.5 hrs of time-lapse microscopy. The 4 myHSCs in WT recipients are representative non-motile cells and had a total displacement <2 μm over 1.5 hrs of imaging. Data are presented as mean±SD *p<0.05; **p<0.01; ***p<0.001.

FIGS. 9A-9C. RNA-Seq analysis of myHSC and balHSC from WT/Sema4AKO mice. (9A) Volcano plots demonstrating that transcriptional changes occur predominantly in Sema4AKO myHSC. (9B) Proliferation-related genes which are upregulated in Sema4AKO myHSC. (9C) Gene set enrichment analysis demonstrating that Sema4AKO myHSC are transcriptionally activated.

FIGS. 10A-10B. (10A) Cell cycle analysis of myHSC 72 hours after a single injection of lipopolysaccharide (LPS) demonstrating exaggerated proliferative response of myHSC in the absence of Sema4A. (10B) MyHSC cell cycle and EdU uptake analysis following 30-day exposure to LPS. In the absence of Sema4A, myHSC undergo premature exhaustion.

### DETAILED DESCRIPTION

Stem cells are unspecialized cells that are able to renew themselves through cell division for long periods. Stem cells can be subdivided and classified on the basis of their potency. A totipotent stem cell is produced from fusion between an egg and a sperm. Stem cells produced by the first few divisions of the fertilized egg cell are also totipotent stem cells. These cells can grow into any type of cell that makes up the developing organism, extra-embryonic tissues (e.g., yolk sac and placenta) and embryo. Pluripotent stem cells are descendants of totipotent cells that can differentiate into any cell type except for placental cells. Multipotent stem cells are descendants of pluripotent stem cells that can produce only cells of a closely related family of cells.

Hematopoietic stem cells (HSC) are pluripotent stem cells that ultimately give rise to all types of terminally differentiated blood cells. HSC can self-renew, or can differentiate into more committed progenitor cells, which progenitor cells are irreversibly determined to be ancestors of only a few types of blood cell. For instance, HSC can differentiate into (i) myeloid progenitor cells, which myeloid progenitor cells ultimately give rise to monocytes and macrophages, neutrophils, basophils, eosinophils, erythrocytes, megakaryocytes/platelets, dendritic cells, or (ii) lymphoid progenitor cells, which lymphoid progenitor cells ultimately give rise to T-cells, B-cells, and lymphocyte-like cells called natural killer cells (NK-cells).

As indicated, the hematopoietic lineage is divided into two main branches: the myeloid arm and the lymphoid arm. The Common Myeloid Progenitor (CMP) gives rise to the myeloid arm, which can give rise to all myeloid cells. The Common Lymphoid Progenitor (CLP) gives rise to the lymphoid arm, which can give rise to all lymphoid cells. Once the stem cell differentiates into a myeloid progenitor cell, its progeny cannot give rise to cells of the lymphoid lineage, and, similarly, lymphoid progenitor cells cannot give rise to cells of the myeloid lineage. For a general discussion of hematopoiesis and hematopoietic stem cell differentiation, see Chapter 17, Differentiated Cells and the Maintenance of Tissues, Alberts et al., 1989, Molecular Biology of the Cell, 2nd Ed., Garland Publishing, New York, NY; Chapter 2 of Regenerative Medicine, Department of Health and Human Services, August 2006 (http://stemcells.nih.gov/info/scireport/2006report.htm), and Chapter 5 of Hematopoietic Stem Cells, 2009, Stem Cell Information, Department of Health and Human Services (http://stemcells.nih.gov/info/scireport/chapter5.asp).

HSC can also be classified as myeloid-biased HSC (myHSC) or balanced HSC (balHSC). myHSC preferentially give rise to myeloid lineage cells through myeloid committed progenitors while balHSC make substantially equal contributions to both myeloid and lymphoid lineages. myHSC are also endowed with a higher self-renewal potential and possess a superior ability to enter the cell cycle in response to inflammatory stress as compared to balHSC (Mann et al., 2018, Cell Rep 25, 2992-3005 e5; Mitroulis et al., 2018, Cell 172, 147-161 e12; and Matatall et al., 2014, Stem Cells 32, 3023-30). myHSC and balHSC can additionally be distinguished based on cell surface markers (myHSC (LKS CD34-CD48-CD150high) and balHSC (LKS CD34-CD48-CD150low) (Beerman et al., 2010, Proc Natl Acad Sci USA 107, 5465-70)), diffusion pseudotime (DPT) analysis, and other gene expression profiles (e.g., vWF, Mp1, Fdg5, Ctnnal1, Procr, Ctsg, and Cbpa).

The current disclosure provides methods and compositions for modulating the myeloid arm of the immune system. The methods and compositions modulate the myeloid arm of the immune system by stimulating or inhibiting Sema4 and/or a PlexinD1 receptor.

Semaphorins are a family of proteins that are divided into seven subclasses. The 7 classes of semaphorins include both transmembrane and secreted proteins and are evolutionarily conserved, structurally and in many cases functionally, from invertebrates to vertebrates (Semaphorin Nomenclature Committee, 1999). The semaphorin family is divided into subclasses based on functional domains and sequence similarity. Rice, D., et al., Severe Retinal Degeneration Associated with Disruption of Semaphorin 4A, Investigative Ophthalmology & Visual Science, Vol. 45, No. 8 (2004). Sema4A is a class IV semaphorin.

Semaphorins were initially identified as axonal guidance cues in the developing nervous system. However, it is now clear that in addition to their central role in the nervous system, semaphorins are implicated in a range of processes, including vascular patterning, tissue morphogenesis, tumor formation, and that they play an important role in the mature immune system. The current disclosure describes a role of semaphorins and particularly Sema4a and Sema3e in regulating the development and activity of the myeloid arm of the immune system.

Reference sequences for Sema4a precursors and proteins can be found at Accession Nos. NP_001357496.1, NP_001180230.1, NP_001180229.1, NP_071762.2, NP_001357500.1, NP_001357498.1, NP_001357497.1, NP_001180231.1, XP_011508174.1, XP_011508173.1, XP_011508180.1, XP_011508177.1, and XP_011508176.1. Recombinant human Sema4a is commercially available, from, for example, Acrobiosystems (Newark, DE) and Sinobiological (Wayne, PA). A reference sequences for Sema3e includes UniProtKB-015041.

Plexins are a family of transmembrane receptors including nine members in vertebrates, divided into four subfamilies: PlexinA (1-4), PlexinB (1-3), PlexinC1 and PlexinD1. Plexins are characterized by an N-terminal 500-amino-acid sema domain that is essential for protein function. The structure of the sema domain is a seven-blade p-propeller fold that has overall structural similarity to the extracellular domain of a-integrins. Next to the sema domain, plexins contain two to three repeats of the plexin-semaphorin-integrin (PSI) domain, a cysteine-rich motif also referred as a MET-related sequence (MRS), and three IPT (Ig-like fold shared by plexins and transcription factors) domains. The cytoplasmic domain of plexins is highly conserved between all members of the family and contains homology to GTPase-activating proteins (GAPs), which catalyze the inactivation of R-Ras GTPase. PlexinD1 is the most divergent plexin family member that includes an atypical feature in its extracellular domain: the third IPT motif in PlexinDI contains only six of the eight conserved cysteins normally encountered in a IPT domain. References sequences for the PlexinD1 receptor include UniProtKB Q9Y4D7 (human) and Q3UH93 (mouse).

Embodiments of the current disclosure encompass methods for modulating the activation state of the myeloid arm of immune system including stimulating or inhibiting Sema4 (e.g. Sema4a) and/or a PlexinD1 receptor (e.g., with Sema4a and/or Sema3e and/or other compounds described herein). Stimulating Sema4 and/or a PlexinD1 receptor down-regulates or dampens the myeloid arm of the immune system while inhibiting Sema4 and/or a PlexinD1 receptor up-regulates or heightens the myeloid arm of the immune system.

As used herein, “down-regulating” or “up-regulating” the activation state of the myeloid arm of the immune system refers to changing the current activation state of the myeloid arm of the immune system in the indicated direction. “Dampening” or “heightening” the myeloid arm of the immune system refers to decreasing or enhancing the responsiveness of the myeloid arm of the immune system to a future event.

Down-regulating or dampening results in more myHSC remaining in a quiescent state whereas during up-regulating or heightening results in more myHSC leaving the quiescent state (i.e, leaving the G0 phase of the cell cycle). Up-regulating or heightening can also lead to more differentiation of myHSC.

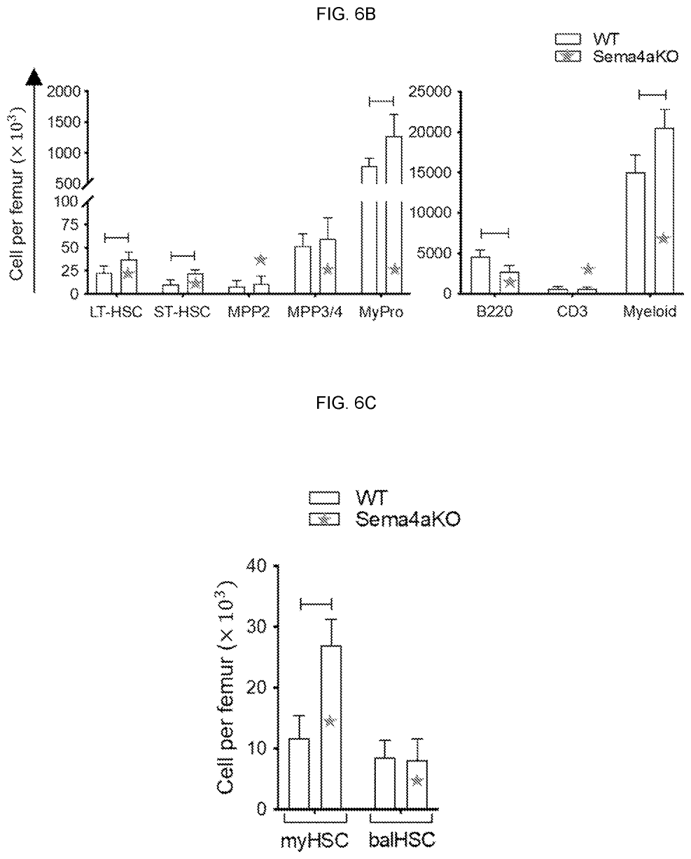

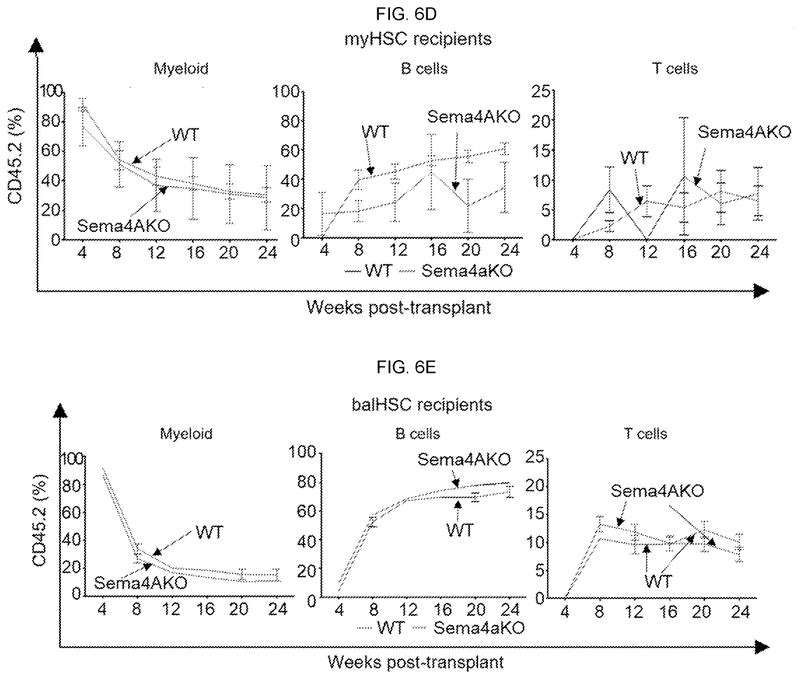

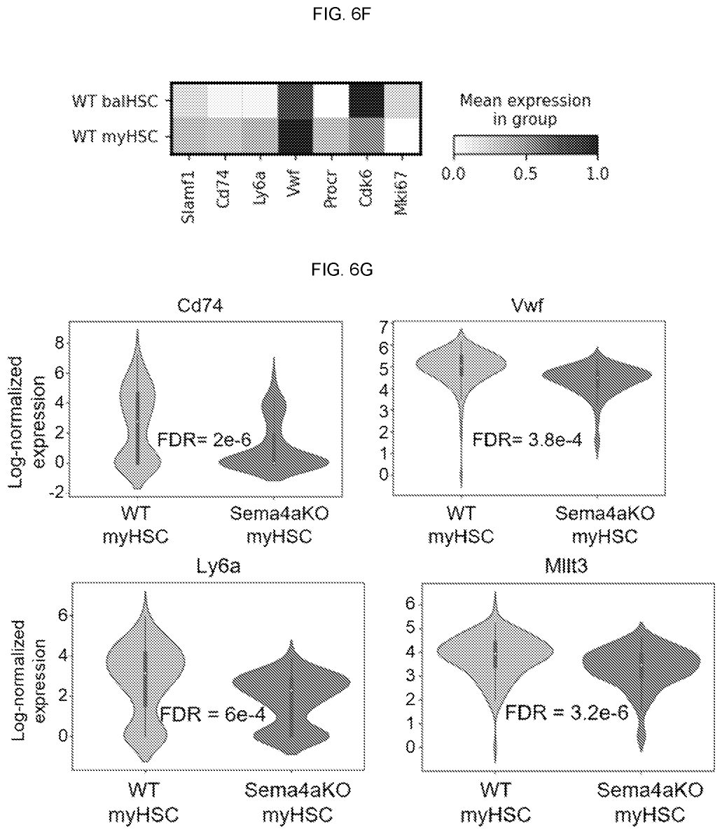

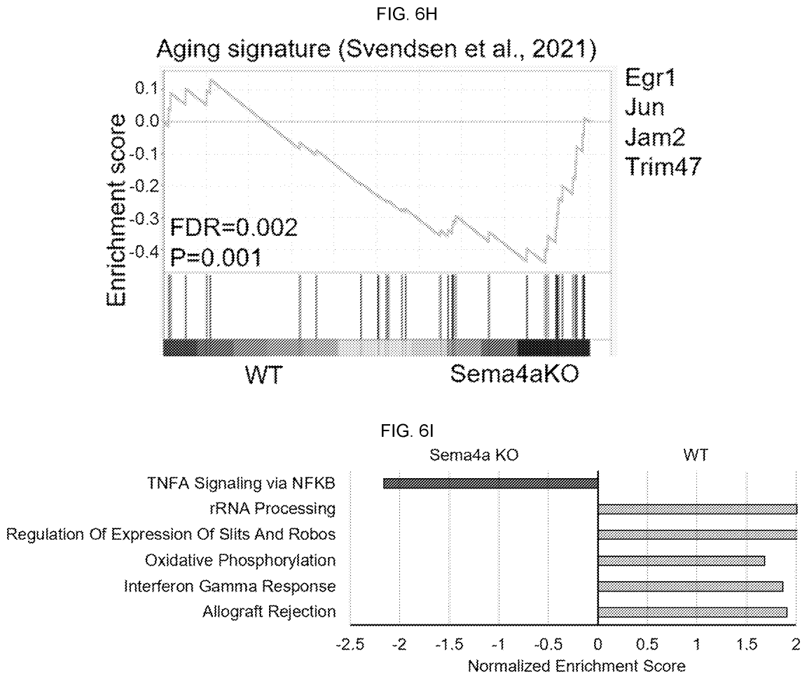

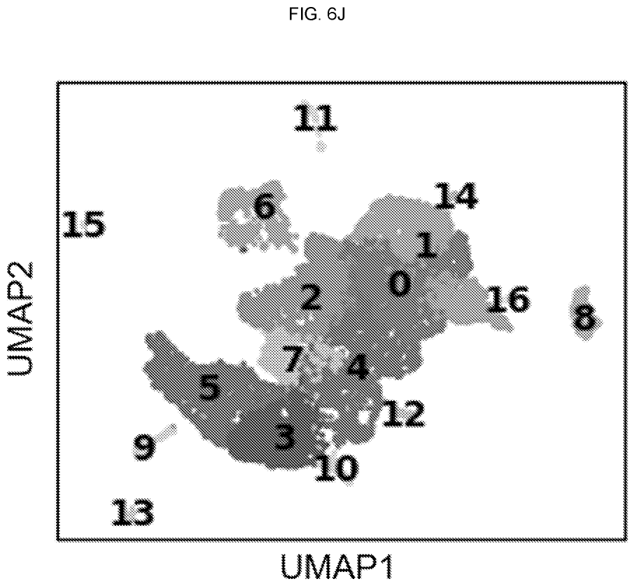

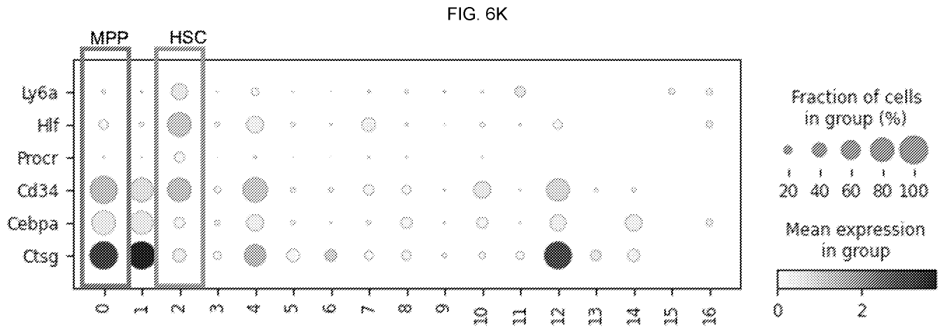

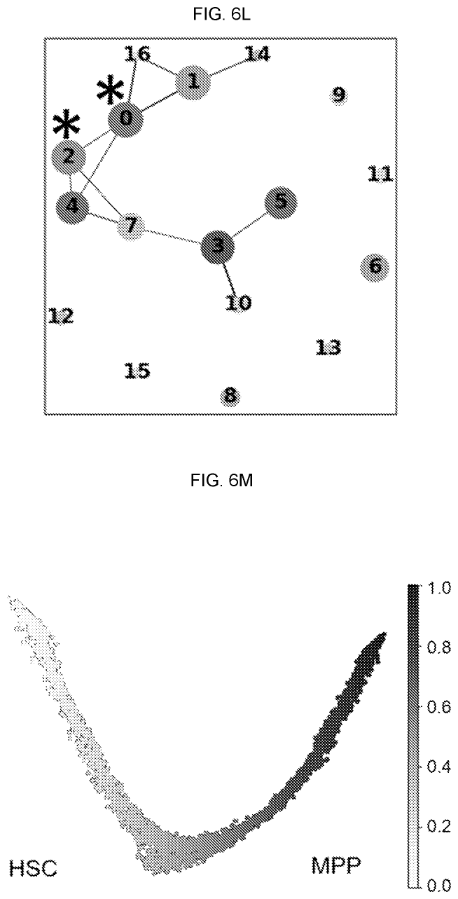

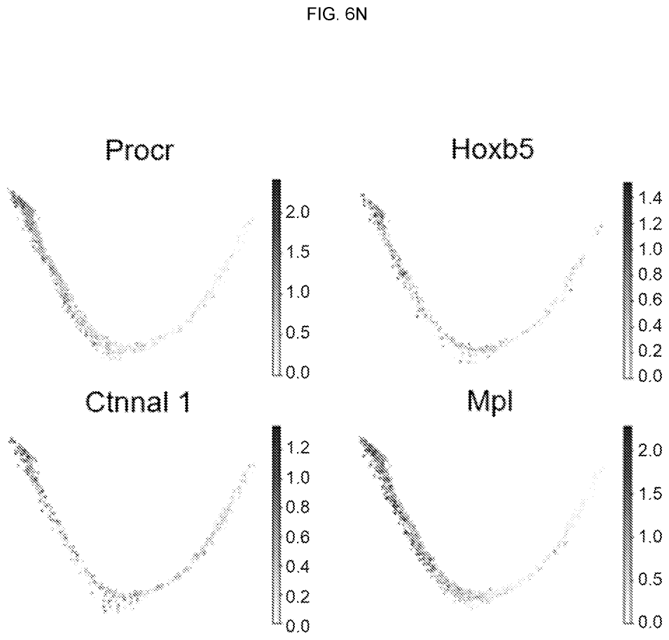

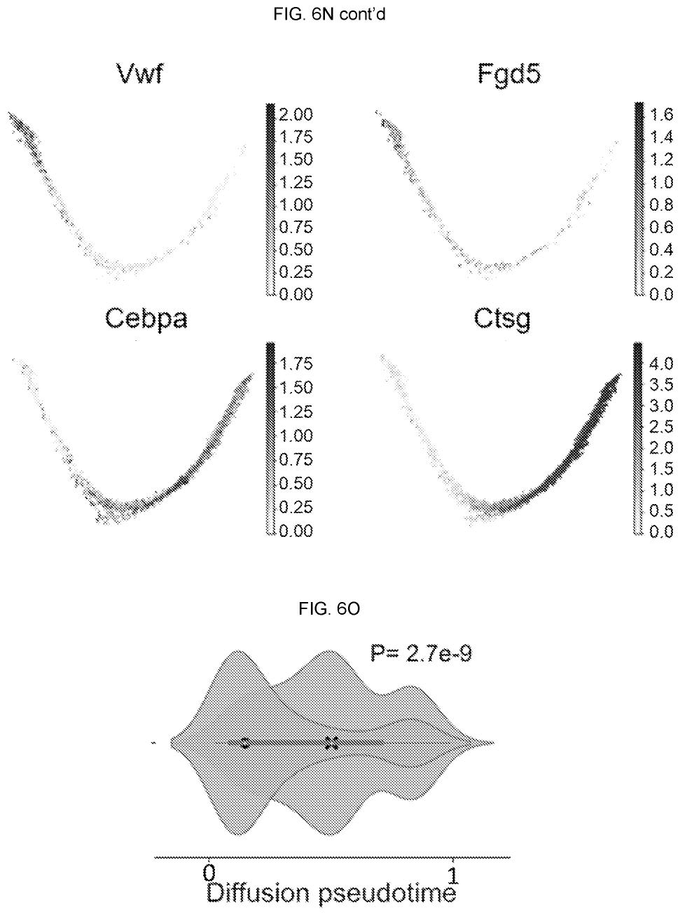

The activation state or responsiveness of the myeloid arm of the immune system to a future event can be measured using a variety of outputs related to HSC quiescence or entry into the cell cycle, indicative of activation and cellular proliferation. For example, gene signatures that constrain the HSC pool and promote self-renewal include CD74, vWF, Ly6a, and Mllt3 (see, e.g., FIG. 6G).

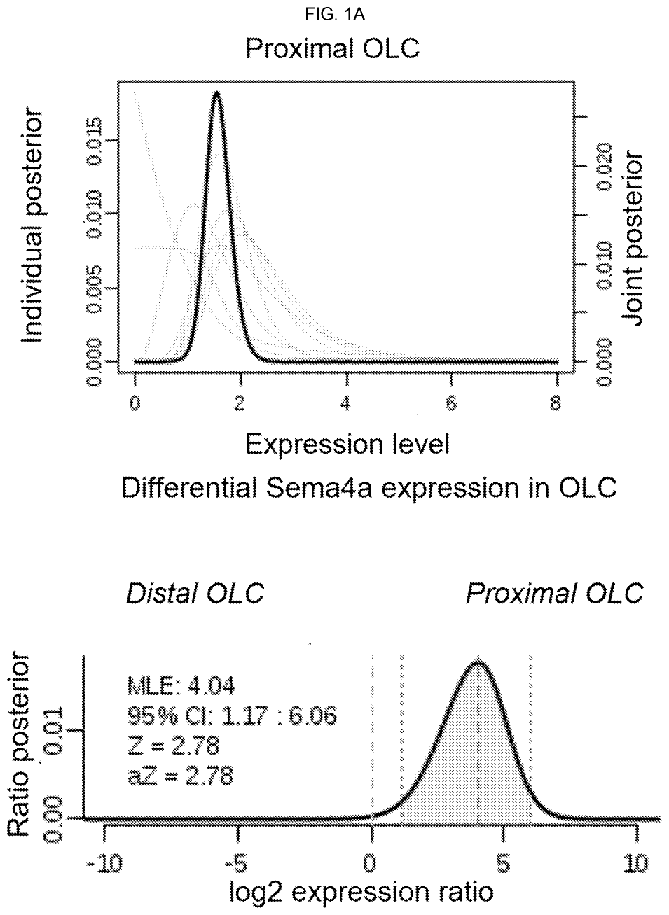

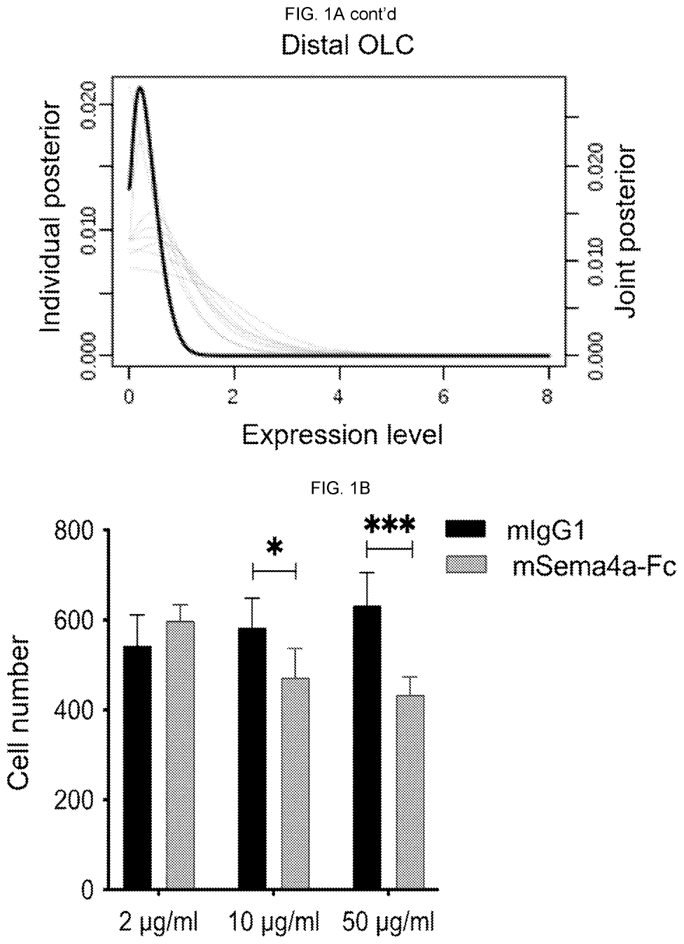

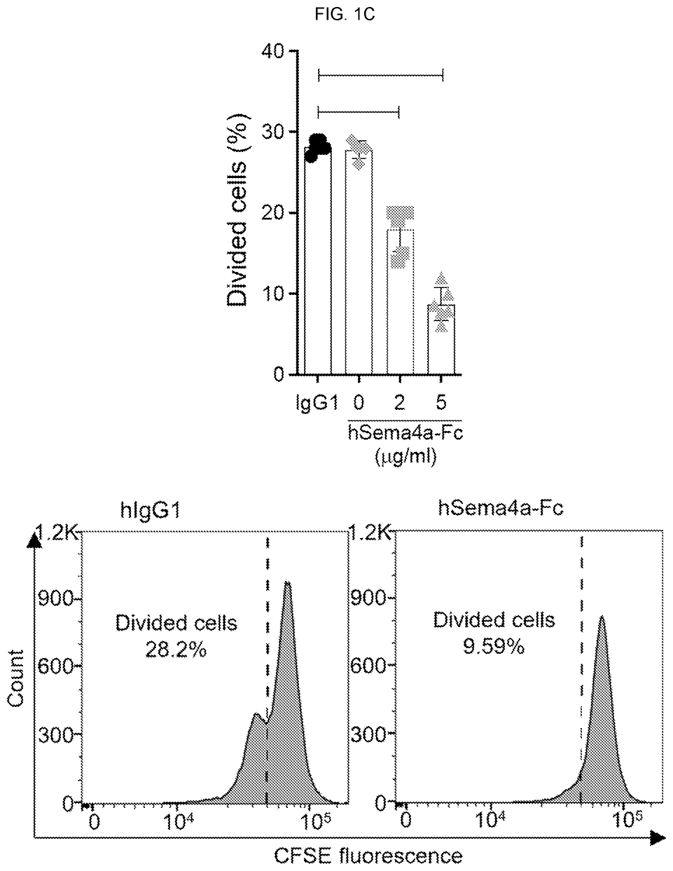

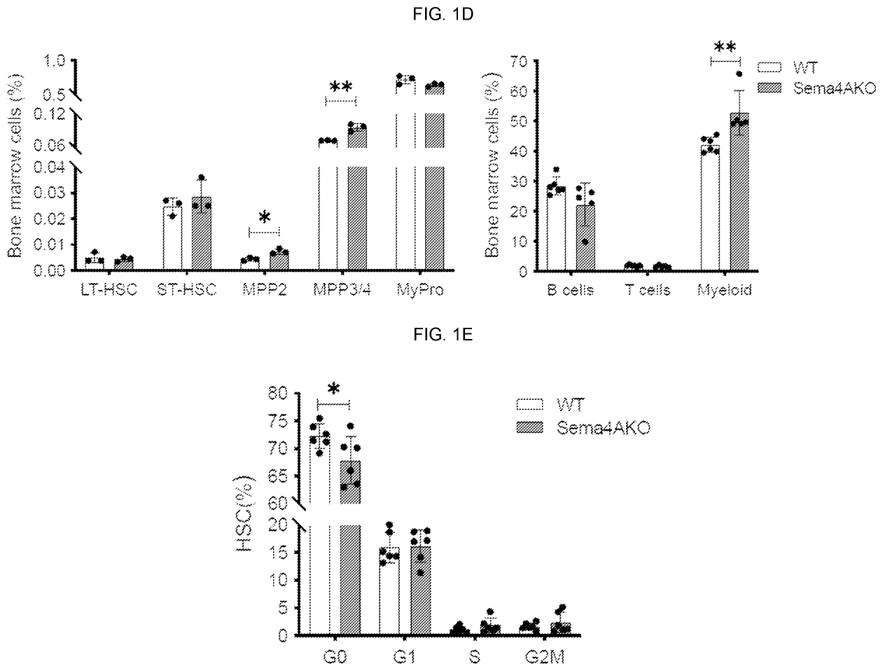

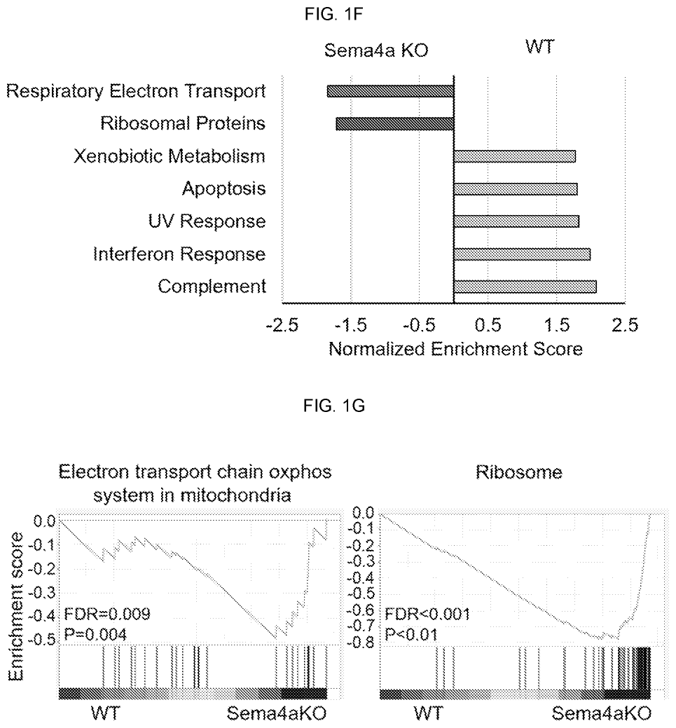

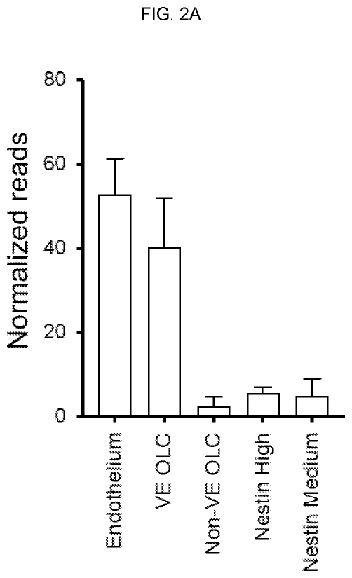

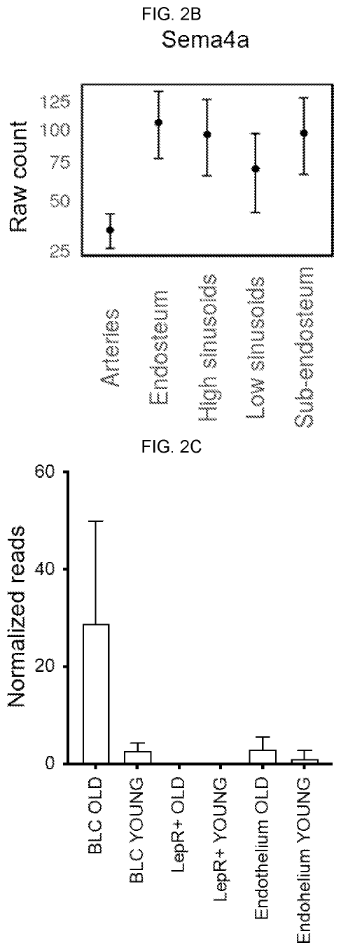

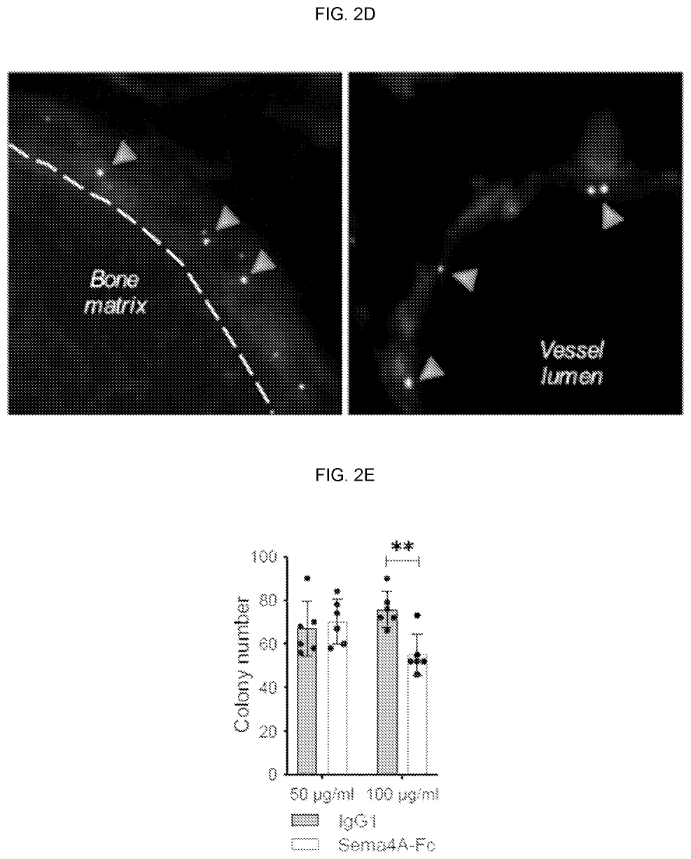

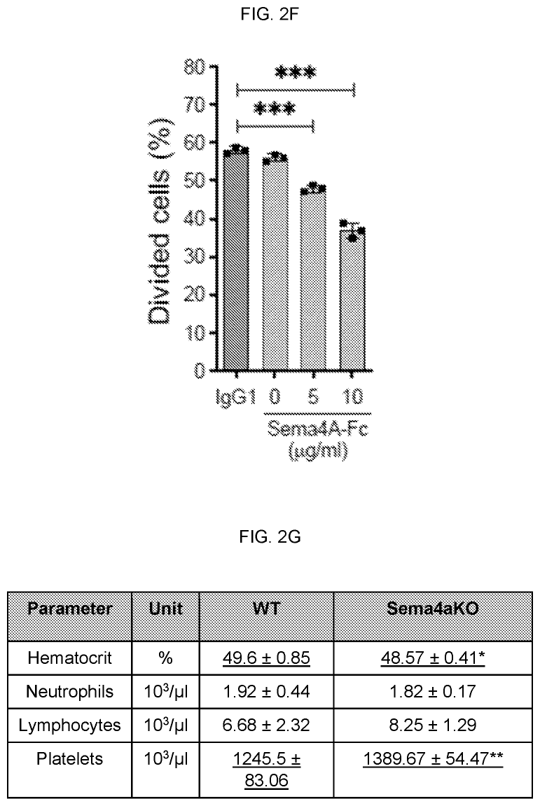

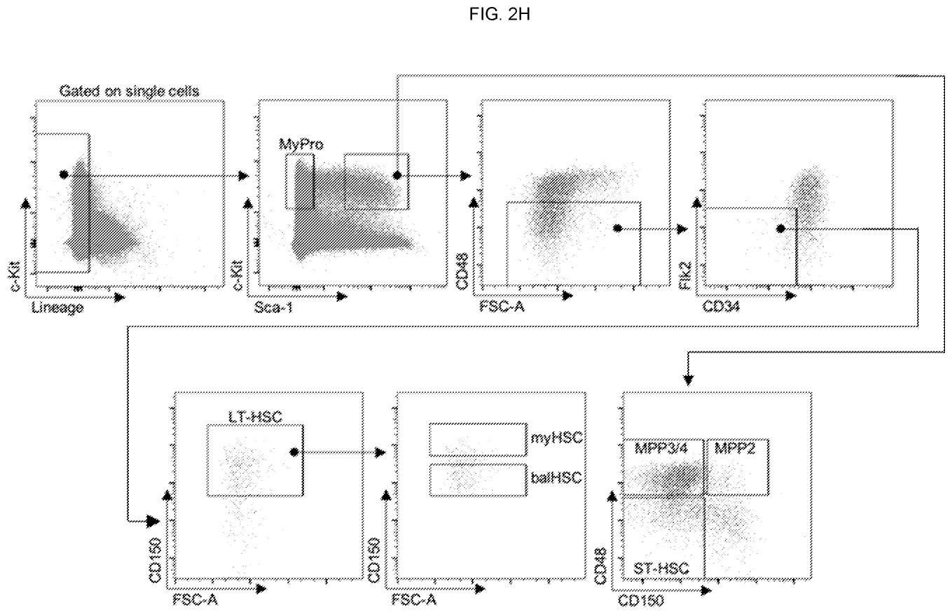

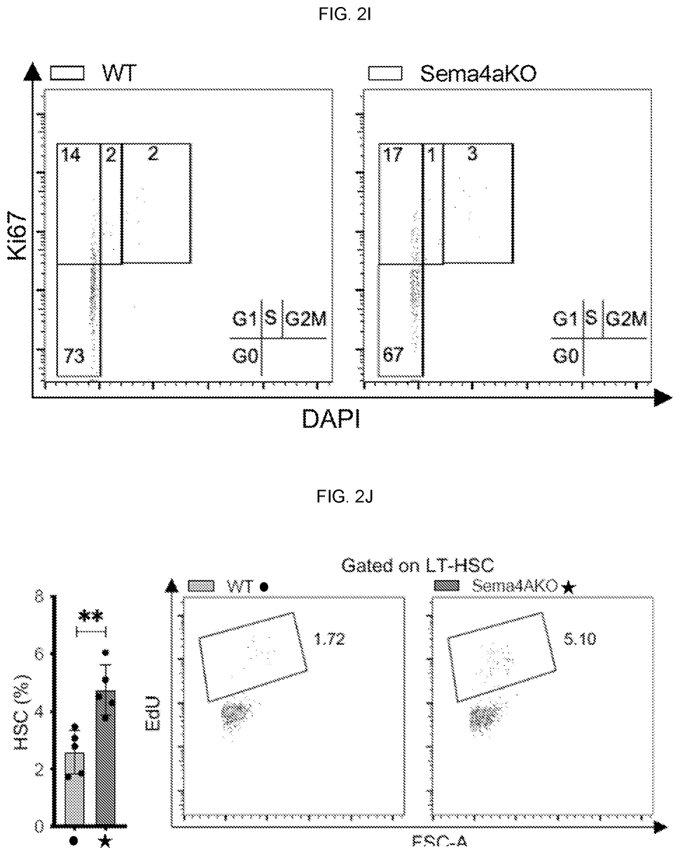

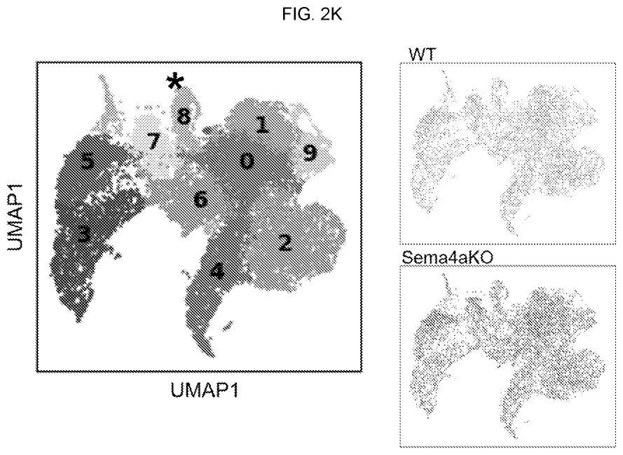

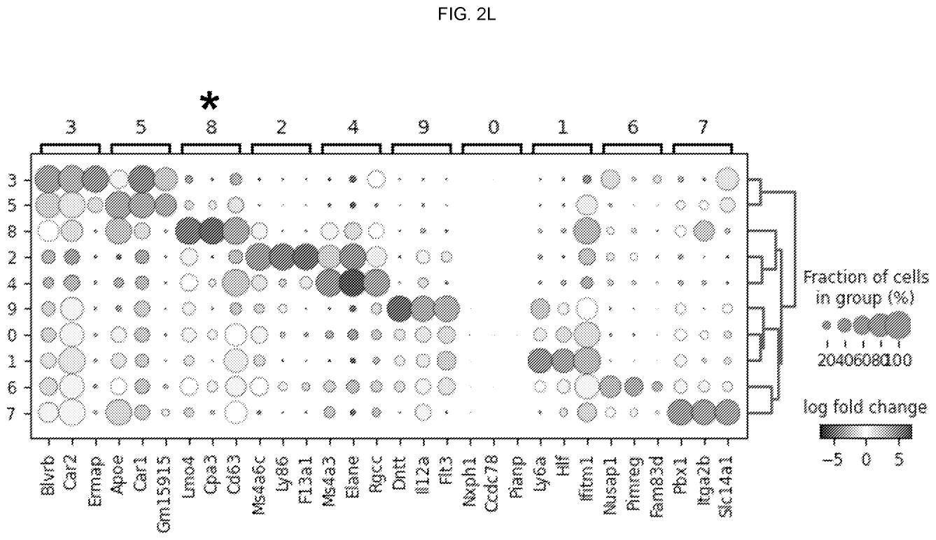

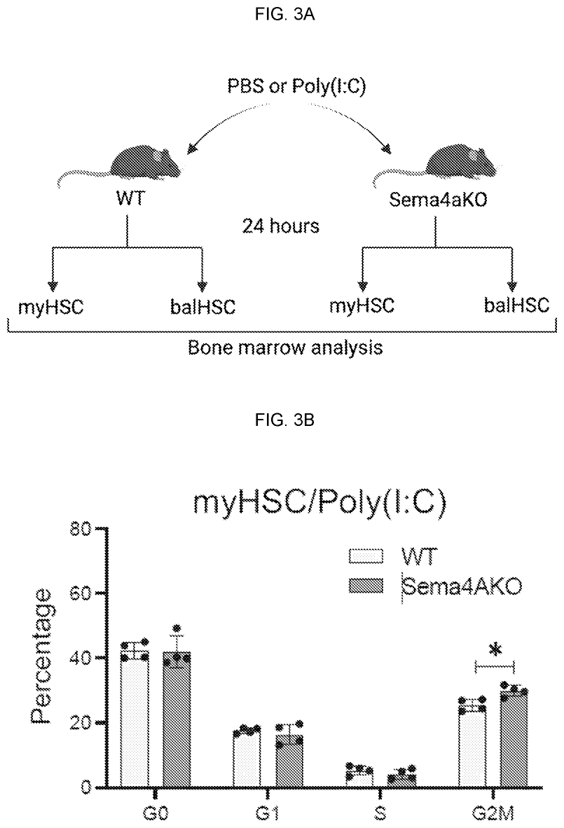

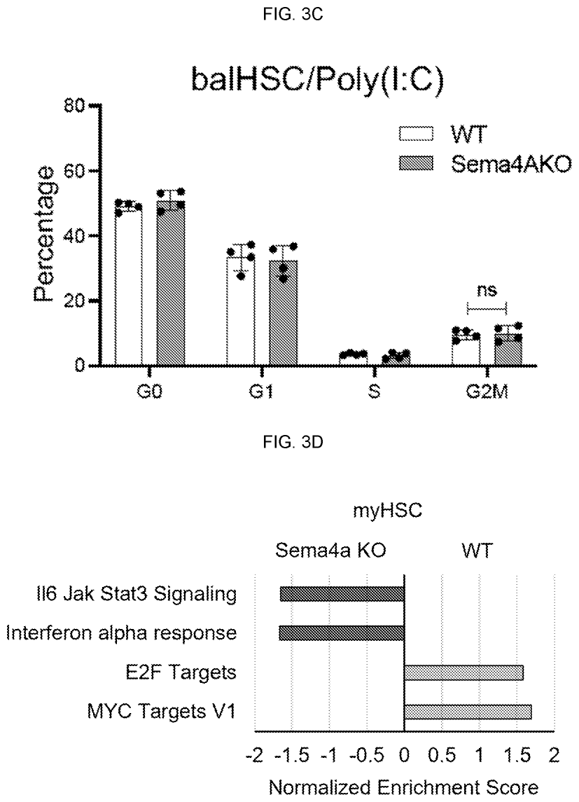

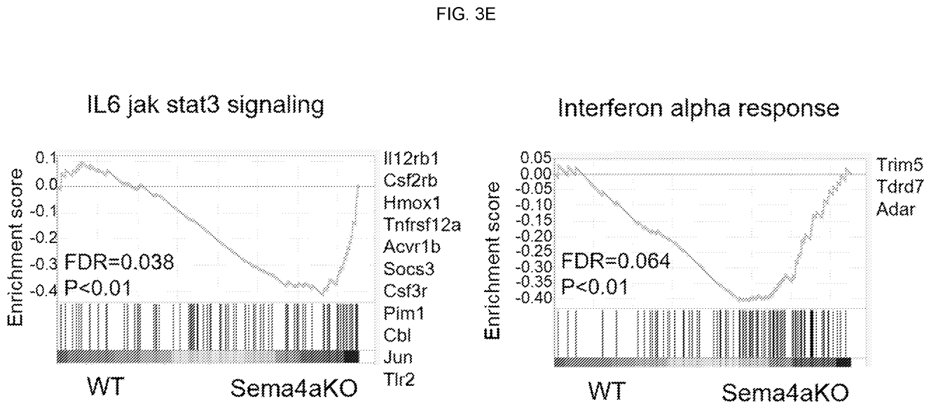

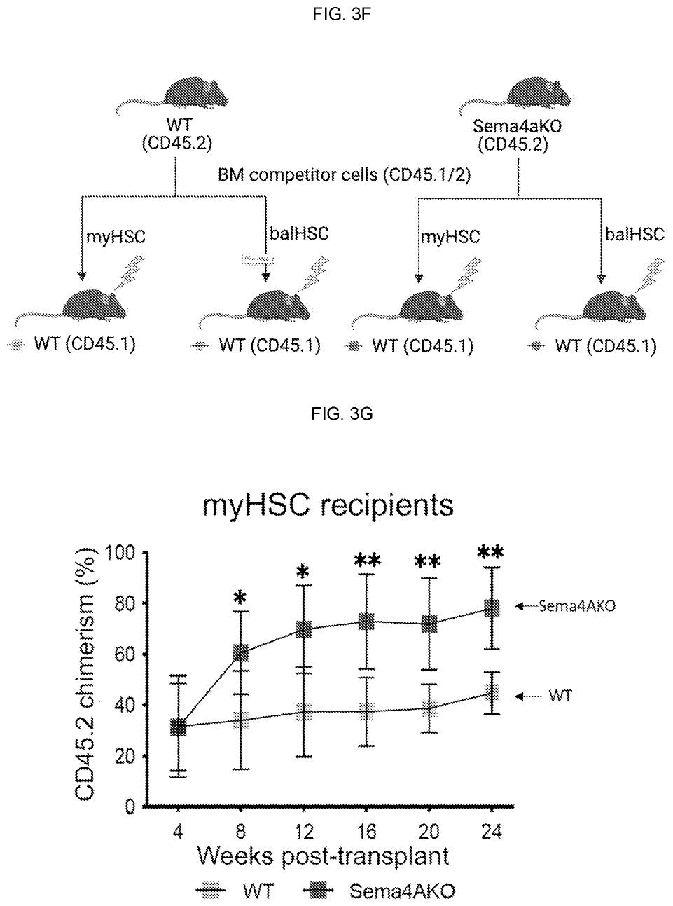

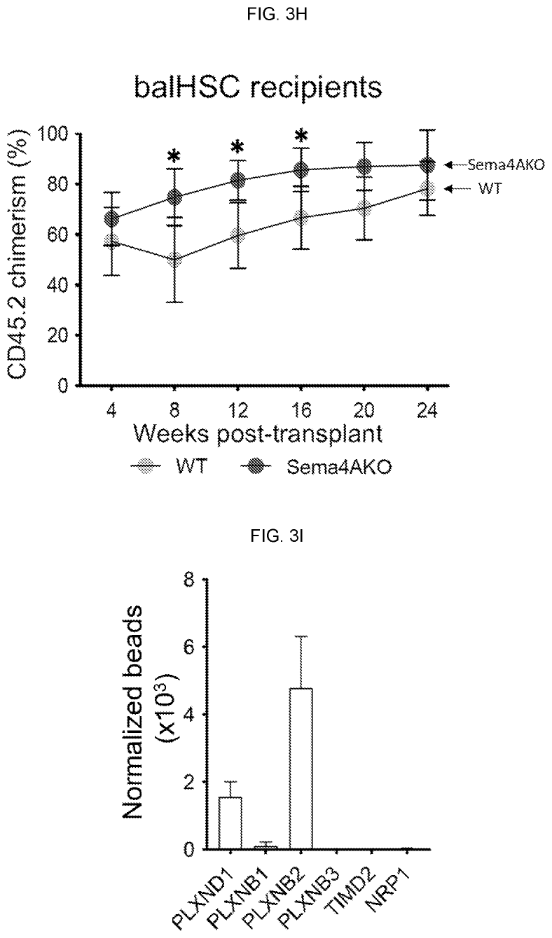

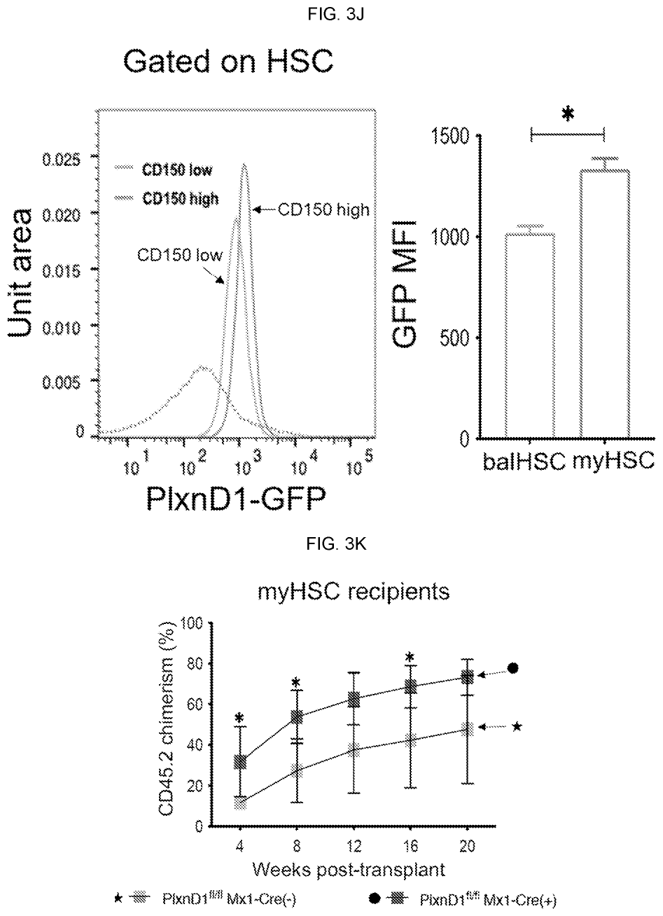

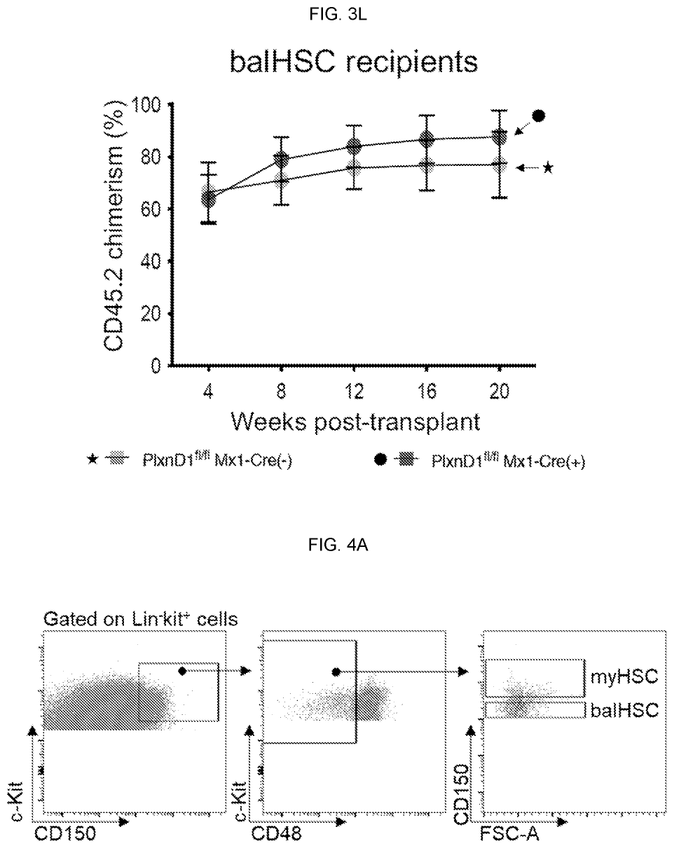

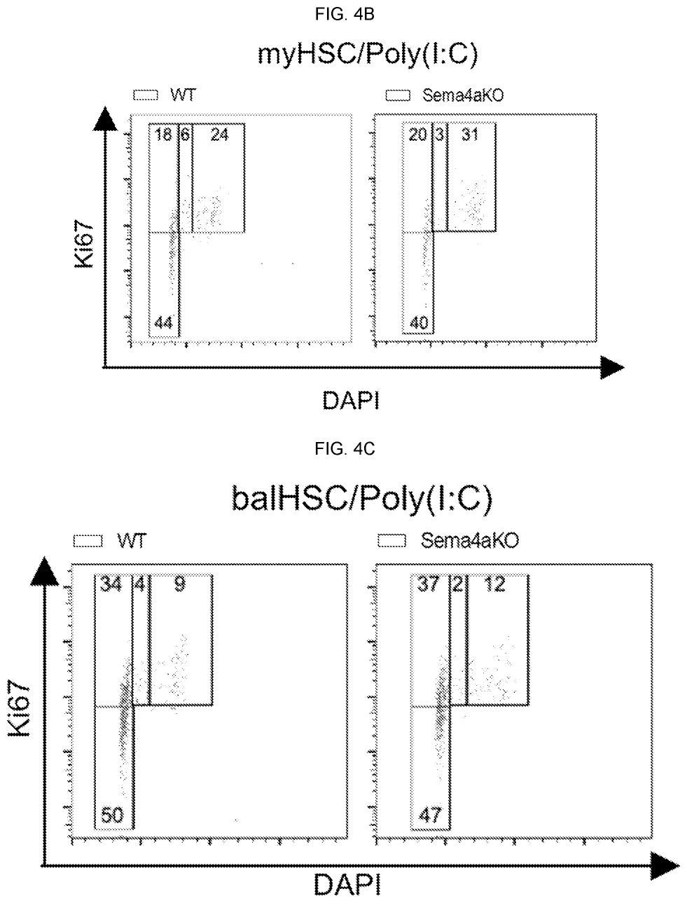

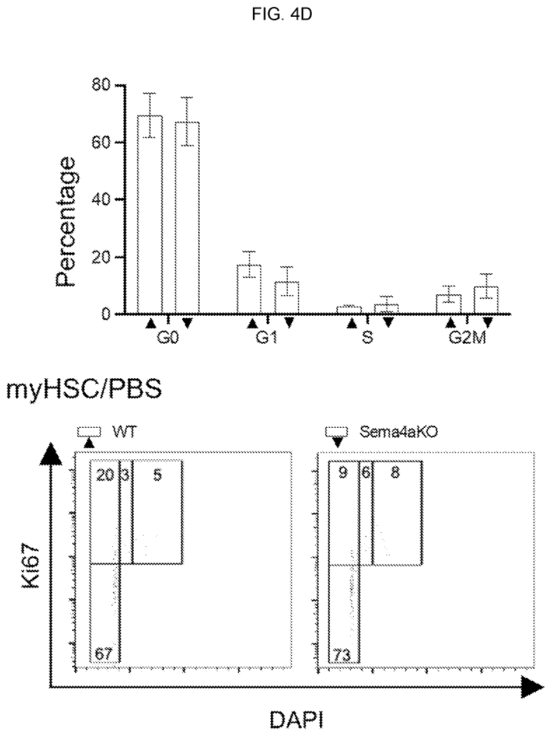

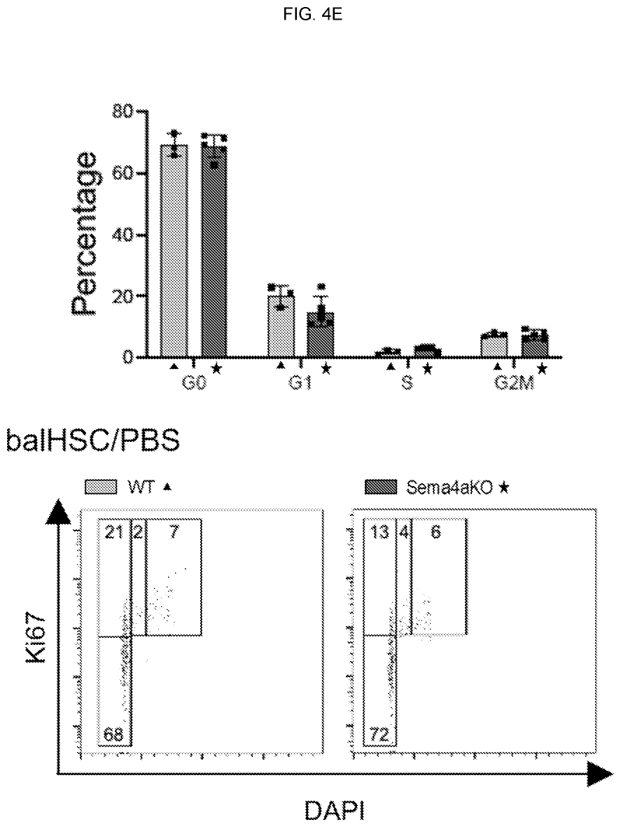

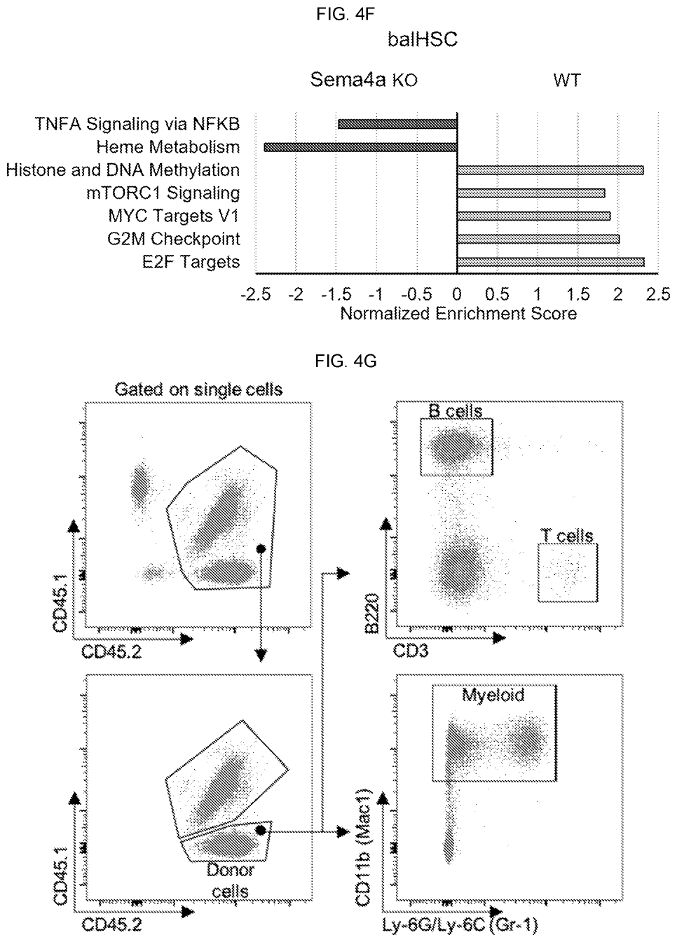

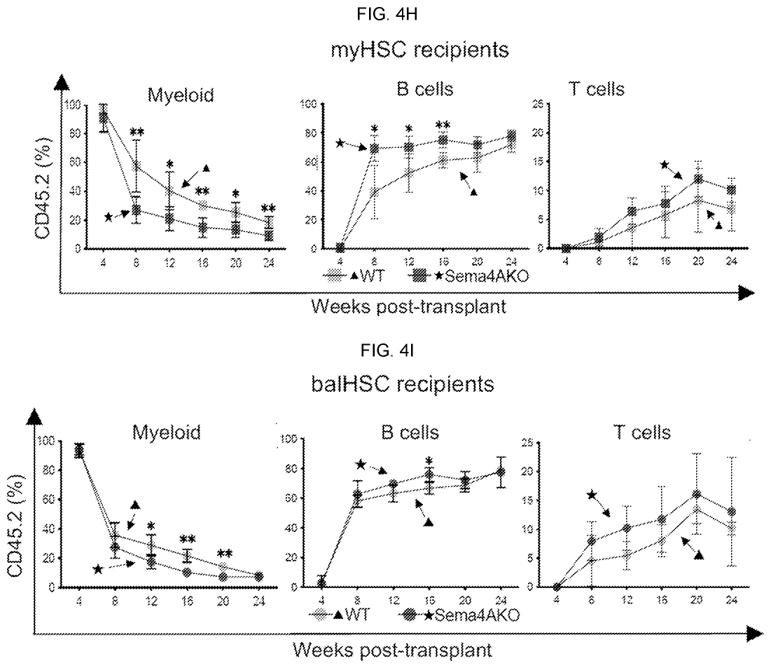

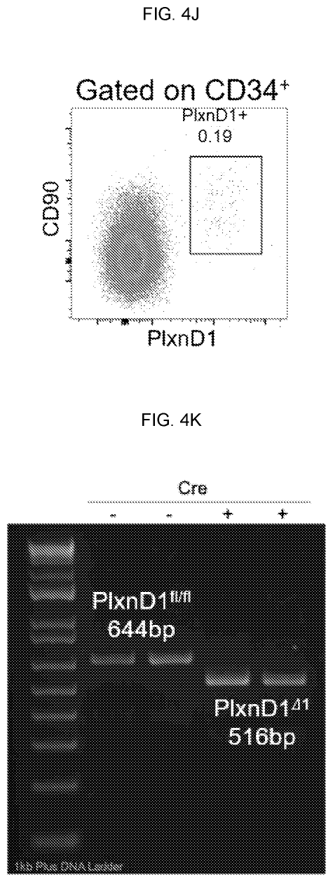

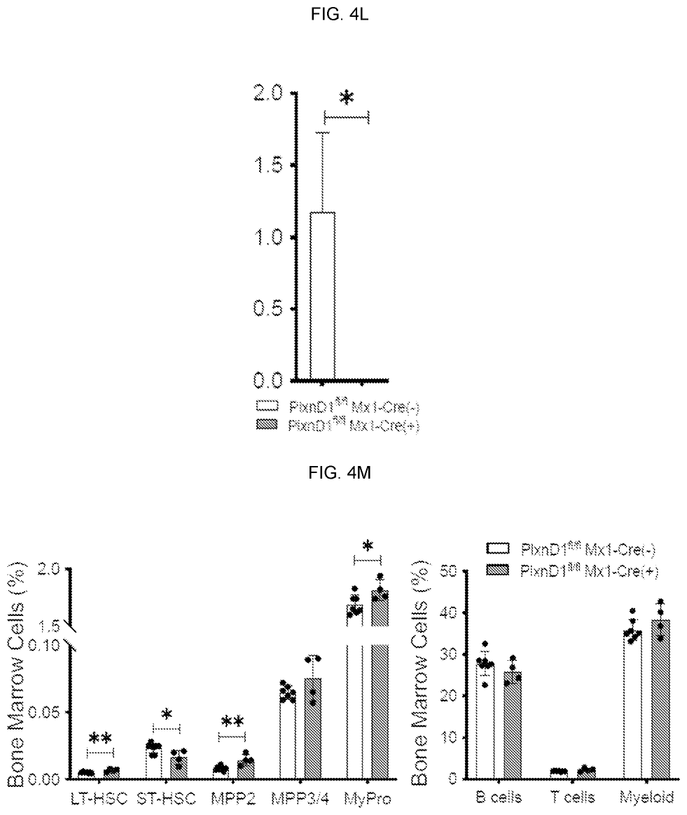

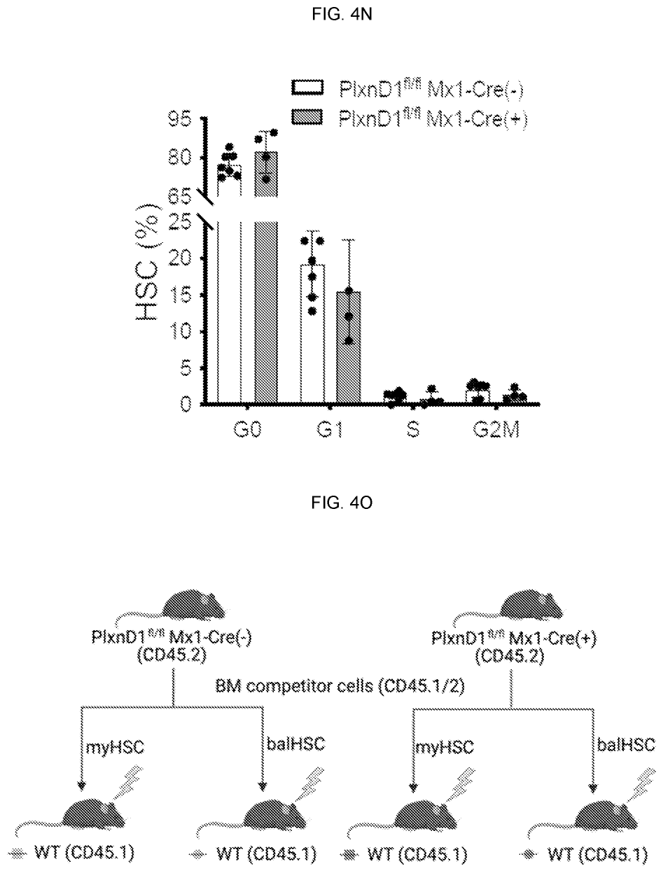

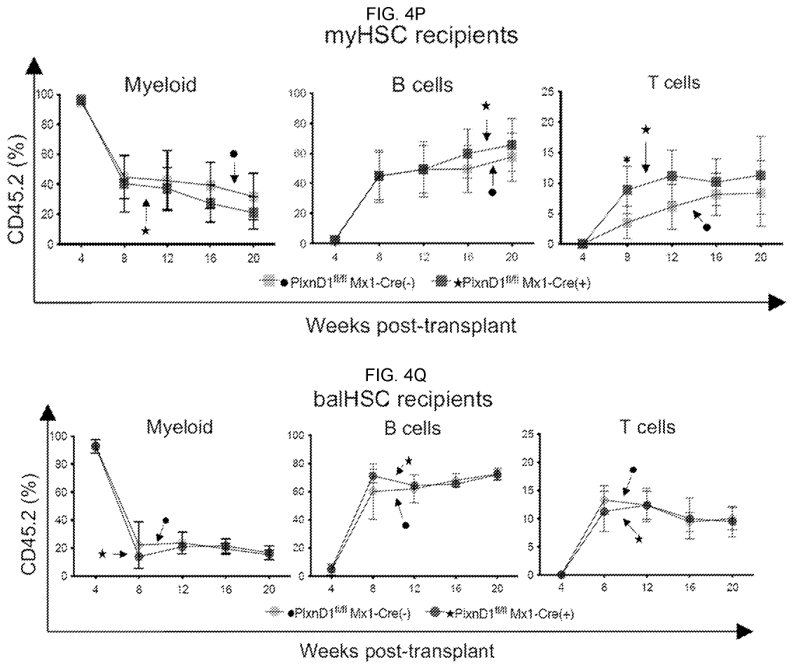

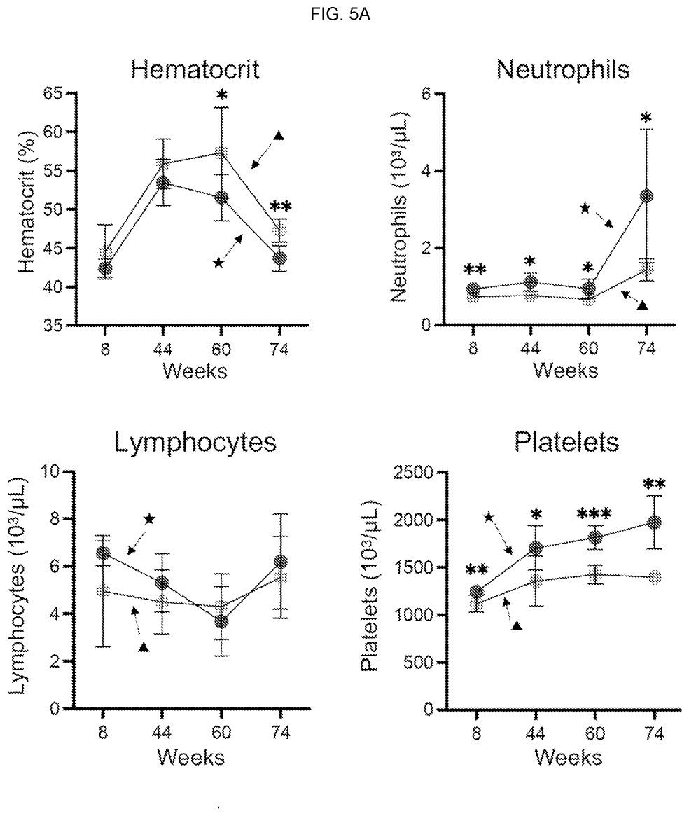

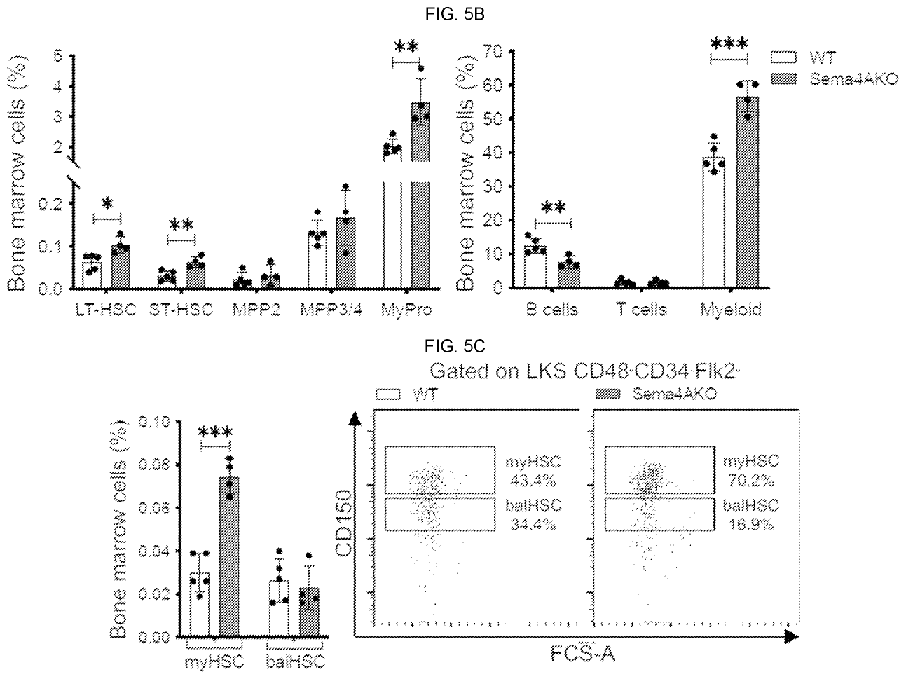

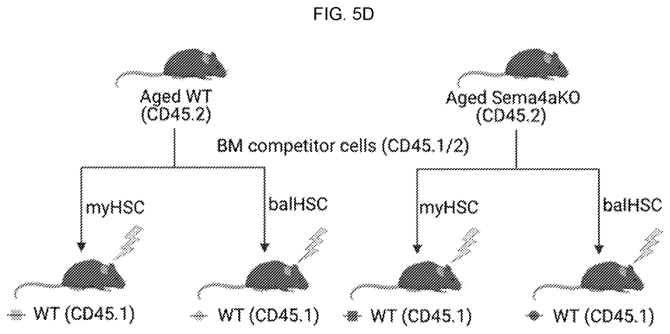

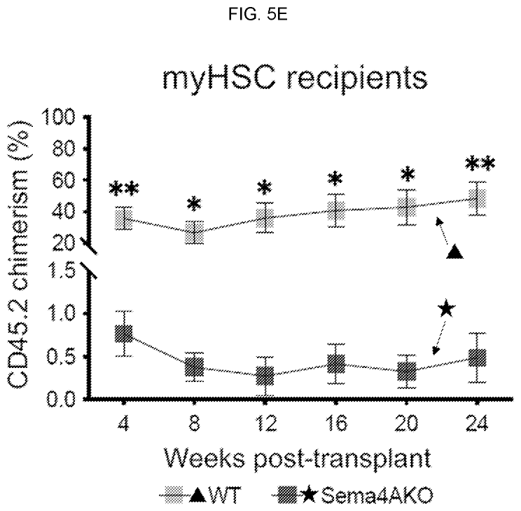

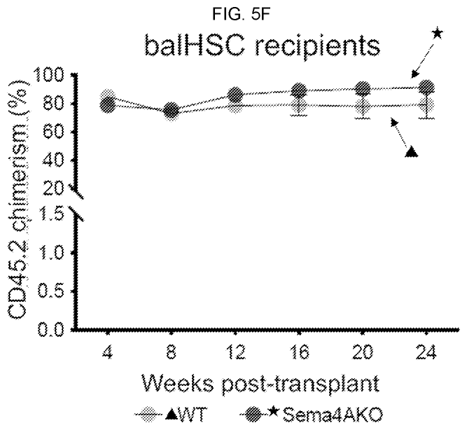

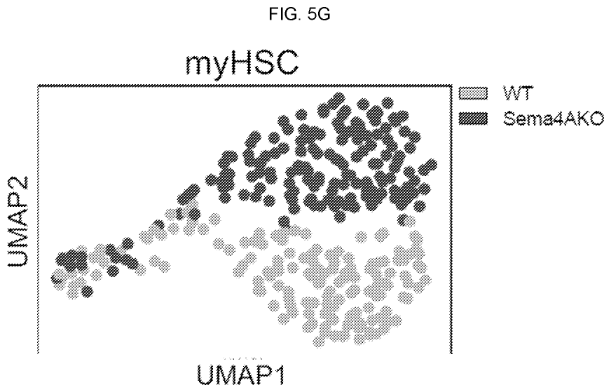

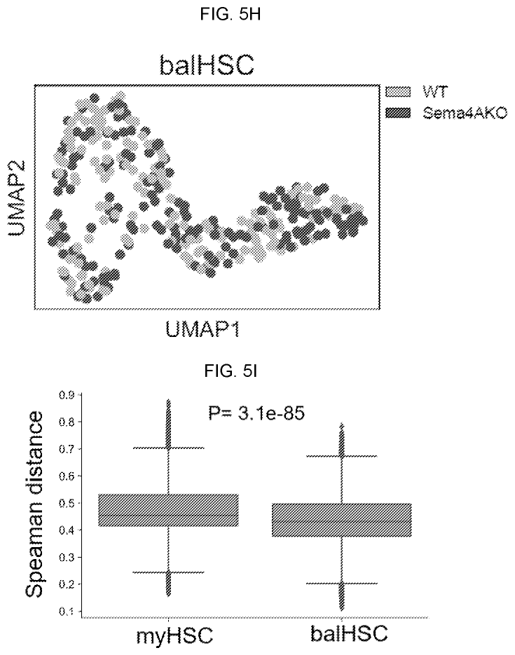

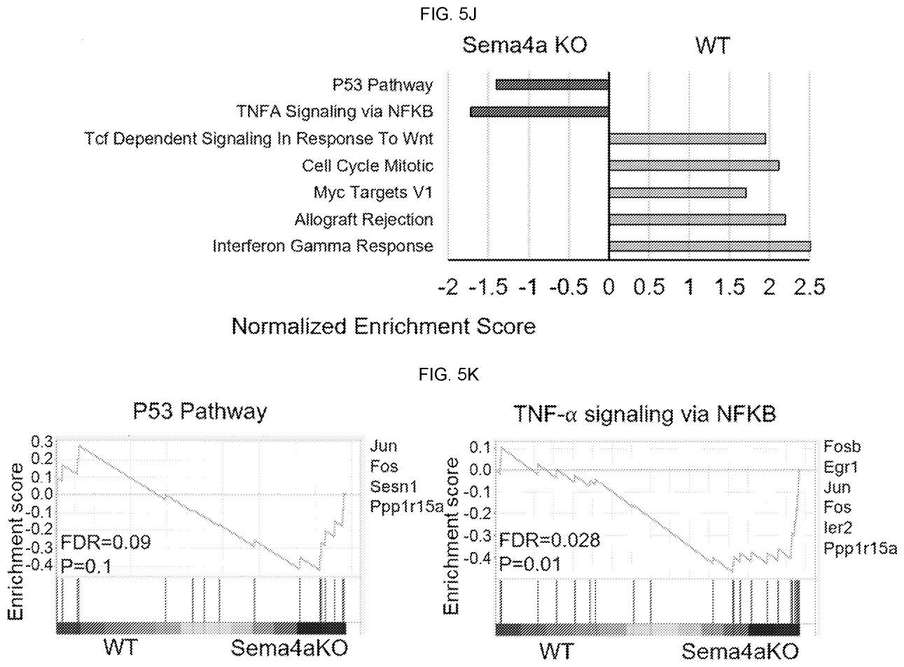

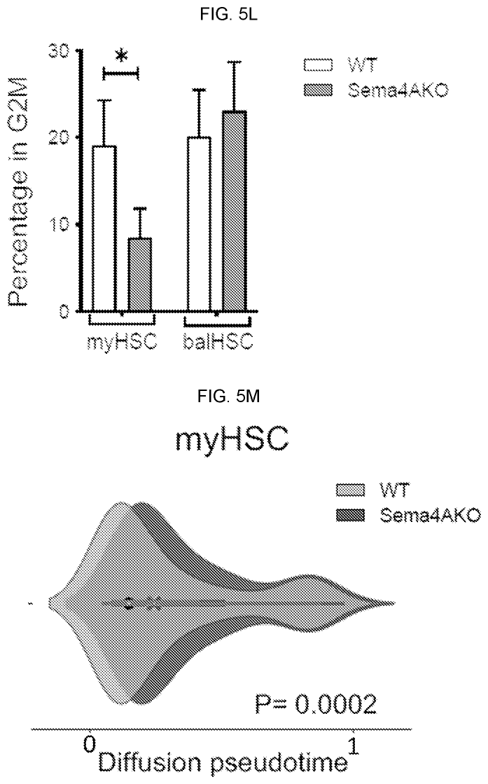

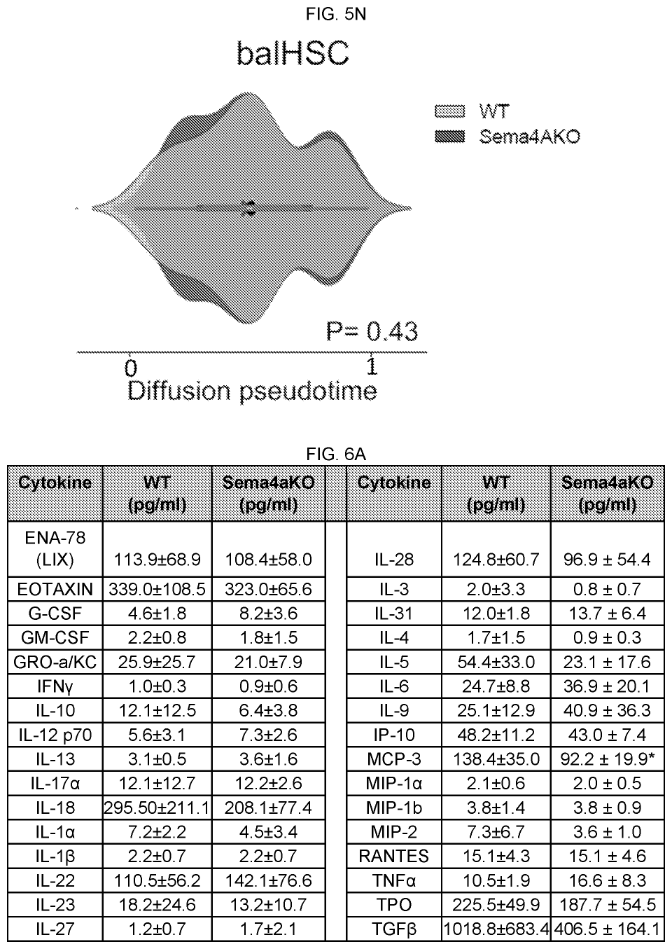

Promotion of quiescence or enhanced or reduced proliferation of HSC can be measured by assessing hematopoietic colony formation (see, e.g., FIGS. 1B, 2E), CFSE dilution assays (see, e.g., FIGS. 1C, 2F), Ki-67/DAPI staining and EdU incorporation (see, e.g., FIGS. 1E, 2I, 2J), percent of myHSC in the G2M phase of the cell cycle (see, e.g., FIGS. 3B, 4B); in silico cell cycle analysis as described in Scialdone et al., 2015 Methods 85, 54-61 (see, e.g., FIG. 5L), and/or by performing RNA-seq analysis for myHSC entry into the cell cycle (enrichment for terms “IL-6-JAK STAT3 signaling” and “interferon response”; see, e.g., FIGS. 3D, 3E, 4F).

Gene signatures associated with a loss of self-renewal capacity and a movement towards differentiation include a down-regulation of vWF, Mpl, Fdg5, Ctnnal1 and Procr and an upregulation of Ctsg and Cbpa (see, e.g., FIG. 6N). Differentiation status can also be assessed using a diffusion pseudotime analysis (DPT) (with an increased DPT score indicative of more differentiation) or by conducting a gene set enrichment analysis (GSEA; see, e.g., FIGS. 5J, 5K, 6H). A loss of self-renewal and subsequent differentiation can be evidence by enrichment for “p53 pathways” (top genes: Jun, Fos, Sesn1), “TNFα/NFKβ signaling” (top genes: Fosb, Egr1, Jun), and the “core aging HSC signature”.

A skew towards the myeloid lineage can be evidence by an increased frequency of MPP2 and mature myeloid cells (see, e.g., FIGS. 1D, 2H).

Methods and compositions disclosed herein also include strategies to modulate the balance of immune cells in the bone marrow and peripheral circulation including stimulating or inhibiting Sema4 (e.g. Sema4a) and/or a PlexinD1 receptor (e.g., with Sema4a and/or Sema3e and/or other compounds described herein). In these examples, stimulating Sema4 and/or a PlexinD1 receptor increases the percent of lymphoid cells and decreases the percent of myeloid cells as compared to before the stimulating. Inhibiting Sema4 and/or a PlexinD1 receptor increases the percent of myeloid cells and decreases the percent of lymphoid cells as compared to before the inhibiting.

As will be well understood by those of ordinary skill in the art, increases and decreases are in relation to relevant control conditions not exposed to a condition as disclosed herein.

Various compounds can be used to stimulate or inhibit Sema4 and/or a PlexinD1 receptor. An exemplary Sema4a activator (or agonist) includes Sema4a-Fc (e.g. recombinant mouse semaphorin 4a Fc chimera protein 4694-S4). An example Sema3e activator (or agonist) includes a Sema3e-Fc (e.g. recombinant mouse semaphorin 3E Fc chimera protein 3238-S3). Recombinant Sema4 and other variants thereof that bind and activate a PlexinD1 receptor can be used. Recombinant protein production methods are well known to those of ordinary skill in the art.

Exemplary Sema4a antagonists include anti-Sema4A antibodies including ab70178, SEMA4A Monoclonal Antibody (5E3) (46-9753-41), SEMA4A Monoclonal Antibody (4E2) (H00064218-M02), SEMA4A Monoclonal Antibody (KL-1 (aka SK31)) (Catalog #14-1002-82), SEMA4A Monoclonal Antibody (4) (Catalog #MA5-30849). Example Sema3e antagonists include anti-Sema3e antibodies including anti-Semaphorin 3E antibody (ab272603), anti-Semaphorin 3E antibody (ab112886), SEMA3E Polyclonal Antibody (Catalog #PA5-56140), Examples of PlexinD1 (PLXDC1) antagonists include PlexinD1 inhibitors, shRNA targeting PlexinD1, or siRNA targeting PlexinD1 (e.g. PLXDC1 small interfering siRNA-incorporated chitosan nanoparticle).

In certain examples, the modulating down-regulates the activation state of the immune system by stimulating Sema4 and/or a PlexinD1 receptor. In certain examples, the modulating dampens the response of the immune system to a proliferative challenge by stimulating Sema4 and/or a PlexinD1 receptor.

The down-regulating or dampening of the immune system response to a proliferative challenge has numerous benefits. For example, the down-regulating and/or dampening can maintain myHSC in a quiescent state, thereby constraining myHSC proliferation, reducing myHSC attrition, reducing myHSC dysfunction, maintaining proliferative function, and/or reducing the risk of malignant transformation. [0041](i) Down-Regulating or Dampening the Myeloid Arm of the Immune System During Ex Vivo Cell Manufacturing. In certain examples, the down-regulating and/or dampening maintains myHSC in a quiescent state during ex vivo cell manufacturing to create a cell population with higher engraftment potential.

Stem cell sources for ex vivo cell manufacturing include umbilical cord blood, placental blood, bone marrow and peripheral blood (see U.S. Pat. Nos. 5,004,681; 7,399,633; and 7,147,626; Craddock et al., 1997; Jin et al., 2008; Pelus, 2008; Papayannopoulou et al., 1998; Tricot et al., 2008; and Weaver et al., 2001), and induced pluripotent stem cells (iPSCs) that can be differentiated into HSCs. Mobilized peripheral blood can also be used. Methods regarding collection, anti-coagulation and processing, etc. of blood and tissue samples are well known in the art. All collected stem cell sources of HSC can be screened for undesirable components and discarded, treated, or used according to accepted current standards at the time.

HSC can be collected and isolated from a sample using any appropriate technique.

Appropriate collection and isolation procedures include magnetic separation; fluorescence activated cell sorting (FACS; Williams et al., 1985; Lu et al., 1986); nanosorting based on fluorophore expression; affinity chromatography; etc. In particular embodiments, it is important to remove contaminating cell populations that would interfere with isolation of HSC, in particular red blood cells.

Culture conditions are selected to maintain HSC with reduced expansion at a time during the culture process. Stimulating Sema4 (e.g. Sema4a) and/or a PlexinD1 receptor (e.g., with Sema4a and/or Sema3e and/or other compounds described herein) can occur at the start of a culture period, after transfection is complete, and/or at a specified time period before administration to a patient. For example, stimulating Sema4 and/or a PlexinD1 receptor can occur 1, 2, 3, 4, or 5 days after transfection is deemed complete. Stimulating Sema4 and/or a PlexinD1 receptor can occur 1, 2, 3, 4, or 5 days before administration to a patient. In certain examples, the culture conditions are those currently used in the art (e.g., with a mix of growth factors and cytokines) with the addition of one or more molecules that stimulates Sema4 and/or a PlexinD1 receptor.

In particular embodiments, disclosed ex vivo manufactured cell populations can provide short- and long-term engraftment for a therapeutic purpose, for example, in the context of cord blood transplants, HSC transplants typically used after radio-ablation of a patient, and other uses.

In particular embodiments, disclosed ex vivo manufactured cell populations can undergo genetic modification for a desired research or therapeutic purpose. “Genetic modification or gene-modifying” means gene disruption, gene editing, gene transfer and any combination thereof. In particular embodiments, gene modification or gene-modifying by gene transfer can be accomplished using any number of DNA or RNA viral vector based or non-viral vector based gene transfer technologies. Examples of viral-mediated gene transfer include lentiviral vectors, foamy viral vectors, adenoviral vectors, adeno-associated viral vectors, alpharetroviral vectors or gammaretroviral vectors; non-viral methods include transposon-mediated, plasmid DNA, nanoparticle delivery, or mRNA delivery using transfection, electroporation or nucleofection.

In particular embodiments, the nucleic acid is stably integrated into the genome of a cell. In particular embodiments, the nucleic acid is stably maintained in a cell as a separate, episomal segment.

Additional gene-editing agents that may be used include transcription activator-like effector nucleases (TALENs), MegaTALs, zinc finger nucleases (ZFNs), and CRISPR-Cas components.

In these embodiments, ex vivo manufactured cell populations can be formulated into cell-based compositions for administration to a subject. A cell-based composition includes ex vivo manufactured cells within a pharmaceutically acceptable carrier. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum or other species serum components.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, cell-based compositions can include a local anesthetic such as lidocaine to ease pain at a site of injection.

Therapeutically effective amounts of cells within ex vivo manufactured cell-based compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In ex vivo manufactured cell-based compositions disclosed herein, cells are generally in a volume of a liter or less, 500 mls or less, 250 mls or less or 100 mls or less. Hence the density of administered cells is typically greater than 10⁴ cells/ml, 10⁷ cells/ml or 10⁸ cells/ml.

The cell-based compositions disclosed herein can be prepared for administration by, for example, injection, infusion, perfusion, or lavage.

Further considerations and procedures for the manipulation, cryopreservation, and long term storage of cells, can be found in the following exemplary references: U.S. Pat. Nos. 4,199,022; 3,753,357; and 4,559,298; Gorin, 1986; Bone-Marrow Conservation, Culture and Transplantation, Proceedings of a Panel, Moscow, Jul. 22-26, 1968, International Atomic Energy Agency, Vienna, pp. 107-186; Livesey and Linner, 1987; Linner et al., 1986; Simione, 1992).

To evaluate engraftment, competitive reconstitution experiments can be performed in the myeloablative, autologous non-human primate NHP stem cell transplant model. Chimerism studies as depicted in relation to FIG. 4G may also be performed.

(ii) Down-Regulating or Dampening the Myeloid Arm of the Immune System to Protect Against Proliferative Challenges. The methods and compositions of the current disclosure can protect against proliferative challenges, such as inflammatory insults, acute inflammatory stress, injury, aging, transplant, and/or the administration of chemotherapy or irradiation. In certain examples, the proliferative challenge is chemotherapy and the down-regulating and/or dampening reduces mutagenesis and risk of development therapy-related myeloid neoplasm (tr-MN).

In certain examples, the methods and compositions of the current disclosure can protect against bone marrow injury, such as age-related clonal hematopoiesis (ARCH). In the face of ARCH, the methods and compositions of the disclosure can slow clonal expansion. In certain examples, the ARCH is clonal hematopoiesis of intermediate potential (CHIP).

The methods and compositions of the current disclosure can also protect against chronic myeloid neoplasms such as myelodysplastic syndrome and myeloproliferative disorders. These disorders are characterized by excessive myeloid expansion which could originate from myHSC.

In certain examples, the proliferative challenge is an adult respiratory distress syndrome. Adult respiratory distress syndromes can be caused by, for example, an infection (e.g., a bacterial, viral (e.g. SARS-CoV-2), fungal, or yeast infection). The down-regulating and/or dampening of the myeloid arm of the immune system can reduce the occurrence of septic shock.

Other hyper-inflammation syndromes characterized by pathological myeloid activation include hemophagocytic syndrome and cytokine release syndrome. Down-regulating or dampening the response of the myeloid arm of the immune system can also serve to protect against these syndromes. [0064](iii) Upregulating or Heightening the Myeloid Arm of the Immune System. While down-regulating or dampening the response of the myeloid arm of the immune system has numerous uses and benefits, in some situations, it is beneficial to up-regulate or heighten the response of the myeloid arm of the immune system. In certain examples, the response of the myeloid arm of the immune system is up-regulated by inhibiting Sema4 (e.g. Sema4a) and/or a PlexinD1 receptor (e.g., with Sema4a and/or Sema3e and/or other compounds described herein). In certain examples, the response of the myeloid arm of the immune system is heightened to a physiological challenge by inhibiting Sema4 and/or a PlexinD1 receptor.

In certain examples, the physiological challenge can include cancer, an infection, or a vaccine. The cancer can be leukemia (e.g., acute myeloid leukemia (AML)). The infection can be bacterial, viral (e.g. SARS-CoV-2), fungal, or yeast. Up-regulating and/or heightening the responsiveness of the myeloid arm of the immune system can increase the clearances of cancers and infections and enhance immune responses to vaccines, as myeloid cells are part of vaccine response.

In particular embodiments, the up-regulating and/or heightening of the myeloid arm of the immune system results in an exit of myHSC from a quiescent state, leaving the G0 phase of the cell cycle.

The current disclosure also provides methods of treating leukemia (e.g., AML) including administering a Sema4a antagonist, Sema3e antagonist, and/or a PlexinD1 receptor antagonist to a subject in need thereof. [0068](iv) Targeting PlxnD1-Expressing Cancer Cells for Cell Killing. Particular examples include selective targeting of PlxnD1-expressing cancer cells (e.g., leukemia cells) using, for example, an anti-PlexinD1 receptor binding domain conjugate. An anti-PlexinD1 receptor binding domain conjugate includes anti-PlexinD1 immunotoxins, anti-PlexinD-drug conjugates (ADCs), anti-PlexinD1-fluorophore conjugates, and anti-PlexinD1 radioisotope conjugates.

Anti-PlexinD1 immunotoxins include an anti-PlexinD1 binding domain conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Antibody-drug conjugates (ADC) allow for the targeted delivery of a drug moiety to a PlexinD1-expressing cell, and, in particular embodiments intracellular accumulation therein, where systemic administration of unconjugated drugs may result in unacceptable levels of toxicity to normal cells (Polakis P. (2005) Current Opinion in Pharmacology 5:382-387).

In particular embodiments, ADC refer to targeted chemotherapeutic molecules which combine properties of both antibodies and cytotoxic drugs by targeting potent cytotoxic drugs to antigen-expressing cancer cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & lyer (Pharm Res. 32(11): 3470-3479, 2015), which describes considerations for the development of ADCs.

The drug moiety (D) of the ADC may include any compound, moiety or group that has a cytotoxic or cytostatic effect. Drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, and stereoisomers, isosteres, analogs, and derivatives thereof that have cytotoxic activity.

The drug may be obtained from essentially any source; it may be synthetic or a natural product isolated from a selected source, e.g., a plant, bacterial, insect, mammalian or fungal source. The drug may also be a synthetically modified natural product or an analogue of a natural product.

Anti-PlexinD1 fluorophore conjugates include a PlexinD1 binding domain linked to a fluorescent label. Fluorescent labels can include any suitable label or detectable group detectable by, for example, optical, spectroscopic, photochemical, biochemical, immunochemical, electrical, optical or chemical means. Exemplary fluorescent labels include blue fluorescent proteins (e.g. eBFP, eBFP2, Azurite); cyan fluorescent proteins (e.g. eCFP, Cerulean, CyPet); green fluorescent proteins (e.g. GFP, GFP-2, EGFP, Emerald) orange fluorescent proteins (mOrange, mKO, Kusabira-Orange, Monomeric); red fluorescent proteins (mKate, mKate2, mPlum, DsRed monomer, mCherry); and yellow fluorescent proteins (e.g., YFP, eYFP, Citrine, SYFP2, Venus, YPet, PhiYFP, ZsYellowl).

Anti-PlexinD1-radioisotope conjugates include a PlexinD1 binding domain linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to PlexinD1 binding domains include iodine-131, arsenic-72, arsenic-74, iodine-131, indium-111, yttrium-90, and lutetium-177, as well as alpha-emitting radionuclides such as astatine-211, actinium-225, bismuth-212 or bismuth-213. Methods for preparing radioimmunoconjugates are established in the art. Anti-PlexinD1 binding domains can also be formed as multi-specific antibodies that bind PlexinD1 and an immune cell activating epitope on an immune cell. Anti-PlexinD1 bispecific antibodies bind at least two epitopes wherein at least one of the epitopes is located on PlexinD1. Anti-PlexinD1 trispecific antibodies bind at least 3 epitopes, wherein at least one of the epitopes is located on PlexinD1, and so on.

In particular embodiments, a bispecific antibody can be in the form of a Bispecific T-cell Engaging (BiTE®) antibody. In particular embodiments, Anti-PlexinD1 binding domains can be used to create bi-, tri-, (or more) specific immune cell engaging antibody constructs wherein the immune cell engaging constructs engages and activates, for example, T-cells, B cells, natural killer (NK) cells, NK-T cells, or monocytes/macrophages to destroy the PlexinD1-expressing leukemia cell.

(v) Compositions for Administration. Compositions for delivery of active compounds can be formulated for administration to a subject in one or more pharmaceutically acceptable carriers. As indicated, salts and/or precursors and other appropriate forms of active compounds can also be used.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A precursor includes an active ingredient which is converted to a therapeutically active compound after administration, such as by cleavage or by hydrolysis of a biologically labile group. In certain examples, a prodrug is a Sema4a precursor.

The formulations disclosed herein can be formulated for administration by, for example, injection. For injection, formulations can be formulated as aqueous solutions, such as in buffers including Hanks' solution, Ringer's solution, saline, buffered saline or physiological saline, or in culture media, such as Iscove's Modified Dulbecco's Medium (IMDM). The aqueous solutions can include formulatory agents such as suspending, stabilizing, and/or dispersing agents.

Examples of suitable non-aqueous carriers which may be employed in the injectable formulations include polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of selected particle size in the case of dispersions, and by the use of surfactants.

Injectable formulations may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like in the compositions.

Compositions can be provided in lyophilized form and/or provided in powder form for constitution with a suitable vehicle, e.g., sterile pyrogen-free water, before use. Lyophilized compositions can include less than 5% water content; less than 4.0% water content; or less than 3.5% water content.

In particular embodiments, the composition can be in a unit dosage form, such as in a suitable diluent in sterile, hermetically sealed ampoules or sterile syringes.

For oral administration, the compositions can be formulated as capsules, dragees, edibles, elixirs, emulsions, gels, gelcaps, granules, gums, juices, liquids, oils, pastes, pellets, pills, powders, rapidly-dissolving tablets, sachets, semi-solids, slurries, sprays, solutions, suspensions, syrups, and tablets. For oral solid formulations such as powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Additionally, compositions can be formulated as sustained-release systems utilizing semipermeable matrices of solid polymers including active ingredients as described herein.

Various sustained-release materials have been established and are well known by those of ordinary skill in the art. Sustained-release systems may, depending on their chemical nature, release active ingredients following administration for a few weeks up to over 100 days. Sustained-release systems can be administered by injection; parenteral injection; instillation; or implantation into soft tissues, a body cavity, or occasionally into a blood vessel with injection through fine needles.

Sustained-release systems can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Therapeutically effective amounts of active ingredients within a composition can include at least 0.1% w/v or w/w active ingredient; at least 1% w/v or w/w active ingredient; at least 10% w/v or w/w active ingredient; at least 20% w/v or w/w active ingredient; at least 30% w/v or w/w active ingredient; at least 40% w/v or w/w active ingredient; at least 50% w/v or w/w active ingredient; at least 60% w/v or w/w active ingredient; at least 70% w/v or w/w active ingredient; at least 80% w/v or w/w active ingredient; at least 90% w/v or w/w active ingredient; at least 95% w/v or w/w active ingredient; or at least 99% w/v or w/w active ingredient.

Certain examples use pharmacological augmentation of Sema4a/PlxnD1 signaling by administering Sema4 (e.g., Sema4a) or Sema3e. PlexinD1 receptor agonists as well as genetic engineering techniques to upregulate expression of Sema4 and/or PlexinD1 can also be used.

Genetic engineering techniques can be based on any appropriate technique including delivery through viral vectors or targeted systems, such as CRISPR, zinc finger nucleases, TALENs, and megaTALs.

Compositions described herein can be packaged into kits for commercial sale. [0096](vi) Additional Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.), for example, with cell-based formulations or compositions, such as those disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide a myeloid-inhibiting effect or a myeloid-potentiating effect. Effective amounts are often administered for research purposes. Effective amounts disclosed herein can cause a statistically-significant effect in an animal model or in vitro assay relevant to the assessment of the functioning of the myeloid arm of the immune system.

A “prophylactic treatment” includes a treatment administered to a subject who does not display signs or symptoms of a condition or displays only early signs or symptoms of the condition such that treatment is administered for the purpose of diminishing or decreasing the risk of developing the condition further. Thus, a prophylactic treatment functions as a preventative treatment against the condition. In particular embodiments, prophylactic treatments reduce, delay, or prevent a cancer or infection from occurring. In particular embodiments, prophylactic treatments reduce, delay, or prevent an adverse effect of a cancer or infection from occurring. Adverse effects can include, for example, therapy-related myeloid neoplasms (tr-MN) and/or respiratory distress syndromes.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

In particular embodiments, therapeutically effective amounts maintain myHSC in a quiescent, G0 phase of the cell cycle. The terms “quiescent” and “G0 phase” as used in the present disclosure refer to specific phases of the cell cycle. The cell cycle is divided into two main parts: interphase and mitosis. During interphase, the cell grows and replicates its chromosomes. Interphase accounts for all but an hour or two of a typical 24-hour cell cycle, and is subdivided into three phases: gap phase 1 (G1), synthesis (S), and gap phase 2 (G2). Interphase is followed by mitosis (known as nuclear division) and cytokinesis (commonly referred to as cell division). This relatively brief part of the cell cycle includes some of the most dramatic events in cell biology. The G1 begins at the completion of mitosis and cytokinesis and lasts until the beginning of S phase. This phase is generally the longest of the four cell cycle phases and is quite variable in length. During this phase, the cell chooses either to replicate its deoxyribonucleic acid or to exit the cell cycle and enter a quiescent state known as the G0 phase. Nonproliferative cells in multicellular eukaryotes generally enter the quiescent G0 state from G1 and may remain quiescent for long periods of time, possibly indefinitely, a common scenario for cells that are fully differentiated. However, also undifferentiated cells such as, interalia, stem cells, progenitor cells or monocytes can reside in the G0 phase of the cell cycle. If such cells are subjected to the right stimulus, for example because they have migrated into a specific part of the body and get in contact with specific cytokines or cells, they may leave the G0 phase to further differentiate or proliferate. During the G0 phase, the cell cycle machinery is dismantled and cyclins and cyclin-dependent kinases disappear. Moreover, quiescent cells reveal low nucleotide levels which accounts for the obstacles a retrovirus has to overcome in order to integrate its genomic information into the genome of a target cell residing in the G0 phase.

In particular embodiments, therapeutically effective amounts provide anti-cancer effects. Anti-cancer effects include a decrease in the number of cancer cells, decrease in the number of metastases, prevented or reduced metastases, a decrease in tumor volume, inhibited tumor growth, an increase in life expectancy, prolonged subject life, induced chemo- or radiosensitivity in cancer cells, inhibited cancer cell proliferation, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, therapeutically effects amounts induce an immune response. The immune response can be against a cancer cell.

Examples of PlexinD1-related disorders include hematological cancers such as leukemias and other myeloproliferative disorders.

Exemplary leukemias include acute lymphoblastic leukemia (ALL), acute myeloid leukemia (AML), chronic myelogenous leukemia (CML), chronic myelomonocytic leukemia (CML), mast cell leukemia, myelodysplastic syndrome (MDS), B-cell acute lymphoblastic leukemia (B-ALL), T-cell acute lymphoblastic leukemia (T-ALL), and megakaryocytic leukemia.

Exemplary sub-types of AML include: acute basophilic leukemia, acute erythroid leukemia (AML-M6), acute megakaryoblastic leukemia (AML-M7), acute monoblastic leukemia (AML-M5a), acute monocytic leukemia (AML-M5b), acute myeloblasts leukemia with granulocytic maturation, acute myeloblasts leukemia without maturation, acute myelomonocytic leukemia (AML-M4), acute panmyelosis with myelofibrosis, acute promyelocytic leukemia (APL), erythroleukemia (AML-M6a), minimally differentiated acute myeloblasts leukemia, myelomonocytic leukemia with bone marrow eosinophilia, and pure erythroid leukemia (AML-M6b).

Certain therapeutically effective amounts disclosed herein provide anti-pathogen effects. In particular embodiments, therapeutically effective amounts provide anti-pathogen effects. Anti-pathogen effects can include anti-infection effects. Anti-infection effects can include a decrease in the occurrence of infections, a decrease in the severity of infections, a decrease in the duration of infections, a decrease in the number of infected cells, a decrease in volume of infected tissue, an increase in life expectancy, induced sensitivity of infected cells to immune clearance, reduced infection-associated pain, and/or reduction or elimination of a symptom associated with the treated infection.

In particular embodiments, therapeutically effective amounts provide anti-inflammatory effects. Anti-inflammatory effects can include reduced inflammation-associated pain, heat, redness, swelling and/or loss of function. Anti-inflammatory effects can also reduce the occurrence of septic shock and/or treat a hyper-inflammation syndrome characterized by pathological myeloid activation. Examples of hyper-inflammation syndrome characterized by pathological myeloid activation include hemophagocytic syndrome and cytokine release syndrome (CRS).

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian, or researcher taking into account parameters such as physical, physiological and psychological factors including target, body weight, stage of condition, type of condition, previous or concurrent therapeutic interventions, idiopathy of the subject, and route of administration.

The compound(s) of the present invention can be administered in any form by any effective route, including, e.g., intra-arterial, oral, parenteral, enteral, intraperitoneal, topical, transdermal, nasally, aerosal, spray, inhalation, subcutaneous, intravenous, intramuscular, intrathecal, intratumoral, etc.

Exemplary doses of active ingredients can include 0.05 mg/kg to 5.0 mg/kg. For certain indications, the total daily dose can be 0.05 mg/kg to 30.0 mg/kg active ingredients administered to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of administration forms of active ingredients using 60-minute oral, intravenous or other dosing. In one particular example, doses can be administered once a day or twice a day (quaque die (QD) or bis in die (BID), respectively) to a subject with, e.g., total daily doses of 1.5 mg/kg, 3.0 mg/kg, or 4.0 mg/kg of a composition with up to 92-98% wt/v of active compound.

Additional useful doses can often range from 0.1 to 5 μg/kg or from 0.5 to 1 μg/kg. In other examples, a dose can include 1 μg/kg, 5 μg/kg, 10 μg/kg, 20 μg/kg, 40 μg/kg, 80 μg/kg, 150 μg/kg, 350 μg/kg, 500 μg/kg, 0.1 to 5 mg/kg or from 0.5 to 1 mg/kg. In other examples, a dose can include 1 mg/kg, 5 mg/kg, 10 mg/kg, 20 mg/kg, 40 mg/kg, 80 mg/kg, 150 mg/kg, 350 mg/kg, 500 mg/kg, 550 mg/kg, 1000 mg/kg, or more.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly). [0115](vii) Control Conditions & Reference Levels. As indicated previously, down-regulation, dampening, up-regulating and heightening can be assessed in relation to relevant control conditions not exposed to a condition resulting in the down-regulation, dampening, up-regulating or heightening. The control condition is identical to conditions resulting in the down-regulation, dampening, up-regulating or heightening, but for exposure to the condition, and with allowance for scientifically reasonable fluctuation in subjects, environmental events, and conditions.

In certain examples, down-regulation, dampening, up-regulating and heightening can be assessed in relation to a reference level. Reference levels can include “normal” or “control” levels or values, defined according to, e.g., discrimination limits or risk defining thresholds, in order to define cut-off points. Other terms for “reference levels” include “index,” “baseline,” “standard,” “healthy,” “pre-disease,” etc. Reference levels can be derived from, e.g., a control subject or population whose myeloid immune activation status is known to be “normal”, “down-regulated”, “dampened”, “up-regulated”, or “heightened”. In some embodiments, the reference value can be derived from one or more subjects who have been exposed to a treatment for, or from subjects who have shown improvements in a condition as a result of exposure to a treatment. In some embodiments the reference level can be derived from one or more subjects who have not been exposed to a treatment.

As used herein, a comparable subject in need thereof is a subject in a similar physiological state as a subject in need thereof, as determined by a treating researcher, veterinarian, or physician. For example, a comparable subject may a share a common diagnosis with a subject and have a common racial and ethnic background. In certain examples, a comparable subject has a common medical history with a subject and is within a common age range with the subject (e.g., previous exposure to chemotherapeutic treatment and within 10 years of age of each other, within 5 years of age of each other, or within 2 years of each other). In other examples a comparable subject and a subject are both enrolled or not enrolled in a clinical trial based on the trial's inclusion and exclusion requirements.

### (viii) EXEMPLARY EMBODIMENTS

1. A method of protecting myeloid-biased hematopoietic stem cells (myHSC) from a proliferative challenge, the method including administering a therapeutically effective amount of a PlexinD1 receptor agonist or a Sema4 (e.g., Sema4a, Sema4b, Sema4c, and/or Sema4d) agonist to the myHSC.
2. The method of embodiment 1, including administering the therapeutically effective amount of the PlexinD1 receptor agonist to the myHSC.
3. The method of embodiment 2, wherein the PlexinD1 receptor agonist includes Sema4 (e.g., Sema4a, Sema4b, Sema4c, and/or Sema4d).
4. The method of embodiment 3, wherein the Sema4 is recombinantly-produced.
5. The method of any of embodiments 1-4, wherein the proliferative challenge includes acute inflammatory stress, injury, aging, transplant, chemotherapy, infection, or irradiation.
6. The method of embodiment 5, wherein the infection includes a bacterial infection, a viral infection, a fungal infection, or a yeast infection.
7. The method of any of embodiments 1-6, wherein the protecting is evidenced by reduced entry of myHSC into the cell cycle as compared to myHSC exposed to the same proliferative challenge in the absence of the therapeutically effective amount of the PlexinD1 receptor agonist or the Sema4 (e.g., Sema4a, Sema4b, Sema4c, and/or Sema4d) agonist.
8. The method of any of embodiments 1-7, wherein the protecting is evidenced by reduced differentiation of the myHSC as compared to myHSC exposed to the same proliferative challenge in the absence of the therapeutically effective amount of the PlexinD1 receptor agonist or the Sema4 (e.g., Sema4a, Sema4b, Sema4c, and/or Sema4d) agonist.
9. A method for maintaining myeloid-biased hematopoietic stem cells (myHSC) in a subject in need thereof including administering a therapeutically effective amount of a composition that signals the Sema4/PlexinD1 receptor signaling pathway in the subject, thereby maintaining the myHSC in the subject in need thereof.
10. The method of embodiment 9, wherein the composition includes Sema4 (e.g., Sema4a, Sema4b, Sema4c, and/or Sema4d).
11. The method of embodiment 10, wherein the Sema4 is recombinantly-produced.
12. The method of any of embodiments 9-11, wherein the subject is in need thereof due to the presence of a proliferative challenge to the myHSC.
13. The method of embodiments 12, wherein the proliferative challenge to the myHSC includes acute inflammatory stress, injury, aging, transplant, infection, chemotherapy, or irradiation.
14. The method of embodiment 13, wherein the infection includes a bacterial infection, a viral infection, a fungal infection, or a yeast infection.
15. The method of any of embodiments 9-14, wherein the maintained myHSC are evidenced by reduced myHSC entry of the myHSC into the cell cycle as compared to myHSC in a comparable subject in need thereof that was not administered the composition.
16. The method of any of embodiments 9-15, wherein the maintained myHSC are evidenced by reduced differentiation of the myHSC as compared to myHSC in a comparable subject in need thereof that was not administered the composition.
17. The method of embodiment 16, wherein the comparable subject in need thereof has a common condition with the subject in need thereof.
18. The method of embodiment 17, wherein the common condition is acute inflammatory stress, injury, transplant, infection, chemotherapy, or irradiation.
19. The method of embodiment 18, wherein the infection includes a bacterial infection, a viral infection, a fungal infection, or a yeast infection
20. The method of any of embodiments 16-19, wherein the comparable subject in need thereof is within an age range of the subject in need thereof.
21. The method of embodiment 20, wherein the age range is within 2 years.
22. A method for modulating the balance of immune cells in the bone marrow of a subject, the method including stimulating Sema4 (e.g., Sema4a, Sema4b, Sema4c, and/or Sema4d) and/or a PlexinD1 receptor in the subject.
23. The method of embodiment 22, wherein the stimulating is through administering a PlexinD1 receptor agonist.
24. The method of embodiment 23, wherein PlexinD1 receptor agonist includes Sema4 (e.g., Sema4a, Sema4b, Sema4c, and/or Sema4d).
25. The method of embodiment 24, wherein the Sema4 is recombinantly-produced.
26. The method of any of embodiments 22-25, wherein the stimulating increases the percentage of lymphoid cells and decreases the percentage of myeloid cells in the bone marrow as compared to before the stimulating.
27. A method of modulating the activation state of the myeloid arm of immune system including stimulating or inhibiting Sema4 (e.g., Sema4a, Sema4b, Sema4c, and/or Sema4d) and/or a PlexinD1 receptor.
28. The method of embodiment 27, wherein the modulating down-regulates or dampens the activation state of the myeloid arm of the immune system by stimulating Sema4 (e.g., Sema4a, Sema4b, Sema4c, and/or Sema4d) and/or a PlexinD1 receptor.
29. The method of embodiment 28, wherein the stimulating is through administering a PlexinD1 receptor agonist.
30. The method of embodiment 29, wherein PlexinD1 receptor agonist includes Sema4 (e.g., Sema4a, Sema4b, Sema4c, and/or Sema4d).
31. The method of embodiment 30, wherein the Sema4 is recombinantly-produced.
32. The method of any of embodiments 28-31, wherein the down-regulating and/or dampening reduces myHSC proliferation and differentiation, as compared to myHSC in the absence of the stimulating.
33. The method of any of embodiments 28-32, wherein the down-regulating and/or dampening reduces myHSC proliferation and differentiation during ex vivo cell manufacturing.
34. The method of any of embodiments 27-33, wherein the modulating dampens a response to a proliferative challenge.
35. The method of embodiment 34, wherein the proliferative challenge includes acute inflammatory stress, injury, aging, transplant, infection, chemotherapy, or irradiation.
36. The method of embodiment 35, wherein the infection includes a bacterial infection, a viral infection, a fungal infection, or a yeast infection.
37. The method of embodiment 27, wherein the modulating up-regulates or heightens the activation state of the immune system by inhibiting Sema4 (e.g., Sema4a, Sema4b, Sema4c, and/or Sema4d) and/or a PlexinD1 receptor.
38. The method of embodiment 37, wherein the heightening increases the response of myHSC to a physiological challenge.
39. The method of embodiment 38, wherein the physiological challenge includes cancer, an infection, or a vaccine.
40. The method of embodiment 39, wherein the cancer is leukemia.
41. The method of embodiment 40, wherein the leukemia is acute myeloid leukemia (AML).
42. The method of embodiment 39, wherein the infection includes a bacterial infection, a viral infection, a fungal infection, or a yeast infection.
43. The method of any of embodiments 37-42, wherein the up-regulating and/or heightening results in increased entry myHSC into the cell cycle as compared to myHSC not exposed to the inhibiting.
44. The method of any of embodiments 37-43, wherein the up-regulating and/or heightening results in increased differentiation of myHSC as compared to myHSC not exposed to the inhibiting.
45. A composition including a therapeutically effective amount of Sema4 (e.g., Sema4a, Sema4b, Sema4c, and/or Sema4d) and a pharmaceutically acceptable carrier.
46. The composition of embodiment 45, wherein the Sema4 is recombinantly-produced.

### (ix) EXPERIMENTAL EXAMPLES

Example 1. A subset of hematopoietic stem cells with inherent myeloid and platelet bias (myHSC) is positioned at the top of hematopoietic hierarchy and considered the most primitive. Notably, lower proliferative output of myHSC correlates with a greater capacity for self-renewal indicating that quiescence is essential for their function. Hence, excessive myHSC expansion following inflammatory challenge is likely to put them at a higher risk of proliferation-induced damage. Given that inflammatory stress is unavoidable throughout life, it was hypothesized that myHSC may uniquely depend on quiescence-inducing signals for their protection and long-term persistence. However, the nature of these signals remains largely unexplored.

Proximity-based analysis of the bone marrow niche to identify novel regulators of HSC quiescence was previously performed and described in Silberstein et al., Cell Stem Cell 2016. Briefly, the transcriptional profiles of osteolineage cells which were located in closer proximity to a transplanted HSC (proximal cells) were defined. Secreted factors with higher expression level in proximal cells as putative regulators of HSC quiescence were also designated. Work described in the current disclosure focused on Semaphorin4a (Sema4a)—a known regulator of neural development, angiogenesis and immune response with no previously documented role in hematopoiesis. Sema4a's impact on myHSC function during inflammatory stress was examined.

As described in this disclosure, Sema4a is expressed in the niche cells (endothelium and osteoprogenitors) in mice and humans. Recombinant Sema4a reduced proliferation of mouse and human hematopoietic stem and progenitor cells ex vivo. Baseline analysis of young Sema4aKO mice revealed mild anemia, thrombocytosis, myeloid bias and a slight reduction in the proportion of HSC in the G0 phase suggesting that Sema4a regulates HSC quiescence and differentiation in vivo. Upon Poly(I:C) injection, Sema4aKO myHSC (Lin⁻Kit⁺Sca⁺CD48⁻CD34⁻CD150^{high}) displayed markedly increased cycling and upregulation of alpha-interferon and JAK-STAT signaling while “balanced” HSC (Lin⁻Kit⁺Sca⁺CD48⁻CD34⁻CD150^{low}) were unaffected. Similar exaggerated proliferative response in Sema4aKO myHSC was observed upon injection with IL-1β.

The long-term impact of inflammation-induced loss of myHSC quiescence was also investigated. Aged Sema4aKO mice developed anemia, thrombocytosis, and neutrophilia. Most significantly, a two-fold expansion of phenotypic myHSC (but not balanced HSC) which displayed proliferative senescence, increased cellular stress and premature differentiation by scRNA-Seq, and a complete loss of reconstitution upon transplantation was observed. In contrast, young Sema4aKO HSC showed a higher level of post-transplant chimerism consistent with their prior “pre-activated” state. Thus, loss of myHSC quiescence leads to increased sensitivity to inflammatory stressors and enhanced myHSC response but eventual collapse of regenerative function.

In order to determine if the microenvironment served as a critical source of Sema4a for myHSC, WT myHSC were transplanted into lethally irradiated WT and Sema4aKO hosts. Strikingly, the majority of Sema4aKO recipients died while all WT recipients survived. Intra-vital imaging at 24 hours revealed a greater number of cells and clusters in Sema4aKO recipients suggesting that excessive early myHSC proliferation led to impaired self-renewal and engraftment failure. Finally, it was found that Plexin D1 acts as a functional receptor for Sema4a on myHSC, since Plexin D1-deficient myHSC recapitulated the post-transplant phenotype of young Sema4aKO myHSC described above.

Taken together, data described herein demonstrates that under the conditions of increased myeloid demand, protection from proliferative stress is critical for preserving myHSC function. This indicates a critical but previously unrecognized role for the Sema4a-PlxnD1 axis in this process. Described studies indicate that therapeutic augmentation of myHSC quiescence can alleviate the negative impact in inflammatory signaling, serving to improve marrow function in inflammatory diseases, and reduce, delay, or prevent development of myeloid malignancy.

This work is described in additional experimental detail in Example 2.

Example 2. Myeloid-biased HSC require Semaphorin 4a from the bone marrow niche for self-renewal under stress and life-long persistence.

Abstract. Tissue stem cells are hierarchically organized. Those that are most primitive serve as key drivers of regenerative response but the signals that selectively preserve their functional integrity are largely unknown. Here, a secreted factor, Semaphorin 4a (Sema4a), is identified as a specific regulator of myeloid-biased hematopoietic stem cells (myHSC), which are positioned at the top of the HSC hierarchy. Lack of Sema4a leads to exaggerated myHSC (but not downstream “balanced” HSC (balHSC)) proliferation after acute inflammatory stress, indicating that Sema4a enforces myHSC quiescence. Strikingly, aged Sema4a knock-out myHSC expand but completely lose reconstitution capacity. The effect of Sema4a is non cell-autonomous, since upon transplantation into Sema4a-deficient environment, wild-type myHSC excessively proliferate but fail to engraft long-term. Sema4a constrains inflammatory signaling in myHSC and acts via a surface receptor PlexinD1. The data presented in this example support the model whereby the most primitive tissue stem cells critically rely on a dedicated signal from the niche for self-renewal and life-long persistence.

Introduction. In multiple tissues, stem cells are hierarchically organized and contain distinct, functionally specialized subsets. For example, in skeletal muscle (Scaramozza et al., 2019, Cell Stem Cell 24, 944-957 e5), cornea (Altshuler et al., 2021, Cell Stem Cell 28, 1248-1261 e8; and Farrelly et al., 2021, Cell Stem Cell 28, 1233-1247 e4), and skin (Rompolas et al., 2013, Nature 502, 513-8; and Hsu et al., 2011, Cell 144, 92-105), the most primitive stem cells (which are also largely quiescent) become activated by injury or stress, whereas their “downstream”, more differentiated counterparts are more proliferative and mainly engaged in on-going tissue repair. Although this two-tiered organization of the stem cell compartment is essential for life-long tissue maintenance, the mechanisms that ensure functional preservation and persistence of individual stem cell subsets within a tissue stem cell hierarchy remain largely unknown.

In the bone marrow, the stem cell hierarchy is exemplified by the myeloid-biased and “balanced” HSC subsets, in which the former is considered the most primitive (Sanjuan-Pla et al., 2013, Nature 502, 232-6; Morita et al., 2010, J Exp Med 207, 1173-82; and Challen et al., 2010, Cell Stem Cell 6, 265-78). Compared to balanced HSC (balHSC), myeloid-biased HSC (myHSC) are inherently skewed towards myeloid differentiation, endowed with a higher self-renewal potential and possess a superior ability to enter cell cycle in response to inflammatory stress (Mann et al., 2018, Cell Rep 25, 2992-3005 e5; Mitroulis et al., 2018, Cell 172, 147-161 e12; and Matatall et al., 2014, Stem Cells 32, 3023-30). Although these properties are beneficial for powerful and timely host defense response, they likely account for myHSC expansion during inflammation and aging, which is associated with profound and irreversible functional loss (Grover et al., 2016, Nat Commun 7, 11075; Pang et al., 2011, Proc Natl Acad Sci USA 108, 20012-7; Esplin et al., 2011, J Immunol 186, 5367-75; and Beerman et al., 2010, Proc Natl Acad Sci USA 107, 5465-70). Thus, despite being positioned at the top of the HSC hierarchy, myHSC appears most vulnerable to stress-induced damage.

In the current example, a membrane-bound and secreted protein Semaphorin 4a (Sema4a) is identified as a myHSC-protective factor. Under stress conditions, such as aging and transplantation, the absence of Sema4a results in excessive expansion and functional attrition of myHSC. Surprisingly, baIHSC are only minimally affected, suggesting that the effect of Sema4a is myHSC-specific. Sema4a from the bone marrow niche is further demonstrated to be essential for myHSC self-renewal and identify PlexinD1 as a functional receptor. These results reveal that by selectively preserving functional myHSC pool, Sema4a plays the key role in long-term maintenance of the HSC hierarchy.

Results. Sema4a regulates quiescence of mouse and human HSPC. Proximity-based analysis has previously been established as a platform for niche factor discovery (Silberstein et al., 2016, Cell Stem Cell 19, 530-543). Single cell RNA-Seq signatures of osteolineage cells (OLC) that were located in close proximity to a single transplanted HSC (proximal OLC) and further away (distal OLC) were compared and several secreted factors with a higher expression in proximal OLC (Angiogenin, IL18, Embigin, VEGF-C) were functionally validated as non cell-autonomous regulators of hematopoietic stem cell/progenitor quiescence (Silberstein et al., 2016, Cell Stem Cell 19, 530-543; Fang et al., 2020, Blood 136, 1871-1883; and Goncalves et al., 2016, Cell 166, 894-906). Because Sema4a displayed a similar expression difference by being significantly more abundant in proximal OLC, as shown in FIG. 1A, it was hypothesized that it would also act as a niche-derived HSC quiescence regulator.

Semaphorins are a large family of membrane-bound and/or secreted proteins which mediate cell-cell communications in neural development, angiogenesis, immune response and cancer (Fard and Tamagnone, 2021, Cytokine Growth Factor Rev 57, 55-63; and Kolodkin et al., 1993, Cell 75, 1389-99). In keeping with this hypothesis for a possible non cell-autonomous regulatory role of Sema4a in hematopoiesis, it was found that Sema4a transcripts were detectable in niche cell subsets, such as CD31+ endothelial cells and niche factor-enriched VCAM1^{high}Embigin⁺ OLC fraction, as supported by the published data (Baccin et al., 2020, Nat Cell Biol 22, 38-48) (FIGS. 2A and 2B). Of note, expression of Sema4a in CD45-Ter119-ALCAM⁺ in bone-lining cells (PMID: 32796847) was significantly increased in aged mice (FIG. 2C). In human bone marrow, Sema4a mRNA was also present in bone-lining and endothelial cells, as demonstrated by single-molecule fluorescent in situ hybridization (FIG. 2D).

In order to gain initial functional insights, the effect of Sema4a on proliferation of mouse and human HSPC in vitro was tested. Consistent with this hypothesis, mouse recombinant Sema4a-Fc protein reduced the number of lin-kit*Sca1+(LKS) after 24 hours of culture and suppressed hematopoietic colony formation in a dose-dependent manner (FIGS. 1B and 2E). Similarly, human recombinant Sema4a-Fc inhibited in vitro proliferation of human bone marrow CD34+ cells from several donors, as measured by Carboxyfluorescein succinimidyl ester (CFSE) dilution assay (Takizawa et al., 2011, J Exp Med 208, 273-84) (FIGS. 1C and 2F).

Taken together, these data indicate that Sema4a non cell-autonomously promotes HSPC quiescence, and that this property is conserved between mice and humans.

Next, the effect of Sema4a on steady-state hematopoiesis was examined using a germline knock-out model. Sema4a knock-out (Sema4aKO) mice are viable and have a normal life span (Kumanogoh et al., 2005, Immunity 22, 305-16). However, baseline analysis of young Sema4aKO animals revealed subtle but reproducible anemia and thrombocytosis (FIG. 2G). Moreover, in the bone marrow, while the number and frequency of long-term HSC (defined as LKS CD34-CD48-CD150+) was similar between the genotypes, the differentiation was skewed towards the myeloid lineage, as evidenced by increased frequency of myeloid-committed progenitor MPP2 and mature myeloid cells (FIG. 1D, see FIG. 2H for gating strategy). Importantly, young Sema4aKO HSC displayed more active cycling, as assessed by Ki-67/DAPI staining and EdU incorporation (FIGS. 1E, 2I and 2J). Consistent with this finding, single-cell RNA-Seq of HSPC from young WT and Sema4aKO mice, while showing no difference in cluster distribution (FIG. 2K), revealed positive enrichment for the terms “Kegg ribosome” and “Electron transport chain oxphos system in mitochondria” in Sema4aKO cells within the HSC cluster by Gene Set Enrichment analysis (GSEA) (FIGS. 1F, 1G, and 2L). Taken together, these results indicate that loss of Sema4a leads to increased HSC proliferation and myeloid-biased differentiation.

Sema4a/PlxnD1 signaling constrains myeloid-biased HSC response to proliferative stress. Given recent evidence suggesting that lineage-restricted HSC subsets are differentially regulated by niche-derived signals, it was hypothesized that myeloid bias in the Sema4aKO model is due to the lack of quiescence-inducing effect of Sema4a specifically on myHSC. While baseline analysis demonstrated no appreciable difference in cell cycle status between Sema4aKO myHSC (LKS CD34⁻CD48⁻CD150^{high}) and baIHSC (LKS CD34⁻CD48⁻CD150^{high}) (Beerman et al., 2010, Proc Natl Acad Sci USA 107, 5465-70) [data not shown], exposure to acute inflammatory stress revealed important HSC subset-specific differences.

In particular, twenty-four hours after injection with polyinosinic:polycytidilic acid (Poly (I:C)) (Walter et al., 2015, Nature 520, 549-52) (FIG. 3A), a significant increase in the percentage of Sema4aKO myHSC in G2M phase of cell cycle was observed (FIGS. 3B and 4B) while no cell cycle difference was observed in the baIHSC subset (FIGS. 3C and 4C, see FIG. 4A for gating strategy under inflammatory conditions) (Hirche et al., 2017, Cell Rep 19, 2345-2356). Of note, myHSC and baIHSC cycling was comparable in PBS-injected WT/Sema4aKO animals (FIGS. 4D and 4E) suggesting that the above changes in Sema4aKO myHSC were due to exaggerated response to inflammatory stress. Indeed, subsequent RNA-Seq analysis of myHSC and balHSC from Poly (I:C)-injected animals revealed enrichment for the terms “IL6-JAK STAT3 signaling” and “Interferon alpha response” which was unique to Sema4aKO myHSC dataset (FIGS. 3D, 3E, and 4F). Thus, these results reveal that the absence of Sema4a promotes myHSC cell cycle entry and lead to enhanced myHSC sensitivity to inflammatory signaling.

The next step was to determine if Sema4a deletion differentially impacts long-term reconstitution capacity of the two HSC subsets. To this end, myHSC and balHSC were isolated from WT and Sema4aKO (CD45.2) donors and each subset was transplanted into lethally irradiated WT (CD45.1) recipients (FIG. 3F). As shown in FIG. 3G, Sema4aKO myHSC displayed a significantly higher level of post-transplant reconstitution as compared to WT myHSC controls, with the difference increasing over time (see FIG. 4G for gating strategy used in chimerism analysis). In contrast, this trend was considerably weaker in the recipients of WT/Sema4aKO balHSC and no longer detectable 24 weeks post-transplant (FIG. 3H). In addition, Sema4aKO myHSC (but not balHSC) graft displayed excessive lymphoid skewing (FIGS. 4H and 4I). These data provide further support for the myHSC-specific action of Sema4a, as evidenced by enhanced output and impaired differentiation of transplanted Sema4aKO myHSC.

In order to establish a cellular mechanism for this effect, a functional receptor for Sema4a on myHSC was searched for. Analysis of published HSC gene expression datasets (Cabezas-Wallscheid et al., 2017, Cell 169, 807-823 e19; and Cabezas-Wallscheid et al., 2014, Cell Stem Cell 15, 507-522) revealed that amongst known receptors for Sema4a, Plexin B2 (PlxnB2) and PlexinD1 (PlxnD1) had the highest expression level in HSC (FIG. 3I). However, PlxnB2 has been described as a receptor for Angiogenin (Yu et al., 2017, Cell 171, 849-864 e25) which has no effect on myeloid differentiation (Goncalves et al., 2016, Cell 166, 894-906). Therefore PlxnD1 was considered the most likely candidate. Interestingly, the ability of Sema4a/PlxnD1 signaling to constrain stress-induced proliferation (as it would be for myHSC) has been already shown for endothelial cells (Toyofuku et al., 2007, EMBO J 26, 1373-84). Furthermore, the analysis of PlxnD1-GFP reporter mice (Gong et al., 2003, Nature 425, 917-25) revealed a significantly higher level of PlxnD1 expression in myHSC as compared to baIHSC (FIG. 3J), which was consistent with a predominant functional effect of Sema4a. Of note, a fraction of CD34+CD90+ human HSC also expressed PlxnD1 (FIG. 4J).

Global deletion of PlxnD1 in mice is embryonically lethal due to structural cardiac and vascular defects (Serini et al., 2003, Nature 424, 391-7), thus precluding functional analysis of adult HSC from these animals. Therefore, PlxnD1 was conditionally deleted by crossing PlxnD1 “floxed” (Zhang et al., 2009, Dev Biol 325, 82-93) mice with the Mx1Cre strain (Kuhn et al., 1995, Science 269,1427-9). A complete excision of PlxnD1 was confirmed by PCR and Q-PCR analysis of sorted LKS cells after Poly (I:C) induction (FIGS. 4K and 4L). Baseline analysis of PlxnD^{flox/flox} Mx1Cre⁺ and PlxnD^{flox/flox} Mx1Cre-mice revealed no significant differences in blood counts, HSC cell cycle and HSPC and mature cell frequency, except for a slight increase in MPP2 and lin-Sca1⁻c-kit⁺ myeloid progenitors, suggesting myeloid bias (FIGS. 4M and 4N). However, competitive transplantation of myHSC and baIHSC from PlxnD1^{flox/flox} Mx1Cre⁺ and PlxnD^{flox/flox} Mx1Cre⁻ mice revealed significantly higher reconstitution by PlxnD1-deficient myHSC while their baIHSC counterparts engrafted normally, thus recapitulating the phenotype of transplanted myHSC and baIHSC from Sema4aKO mice (FIGS. 3K-3L and 4O-4Q). In sum, the results are consistent with a previously unrecognized role of PlxnD1 as a functional receptor for Sema4a on myHSC.

Sema4a prevents excessive myHSC expansion and functional loss with age. The observation that Sema4a loss enhances myHSC responsiveness to proliferative challenges, such as acute inflammation and transplantation, prompted the investigation of whether this will lead to impaired myHSC function upon chronic inflammatory stimulation, as occurs during aging (Kovtonyuk et al., 2016, Front Immunol 7, 502). Analysis of peripheral blood in aged Sema4aKO mice revealed progressive anemia, thrombocytosis, and neutrophilia (FIG. 5A). In order to rule out systemic inflammation as a cause of the above abnormalities, plasma levels of 36 proinflammatory cytokines were examined in aged WT and Sema4aKO mice (including thrombopoietin and G-CSF) but no significant differences were detected (FIG. 6A).

In the bone marrow of aged Sema4aKO mice, a higher number of primitive hematopoietic cells and marked myeloid expansion was detected, as evidenced by increased frequency and absolute number of myeloid progenitors and mature myeloid cells (FIGS. 5B and 6B). Critically, a marked (2.5-fold) increase in the absolute number of myHSC was observed while the number of baIHSC was comparable with that of aged-matched WT controls (FIGS. 5C and 6C).

The amplified Sema4aKO myHSC population may represent either expanded bona fide myHSC or a more differentiated progeny which retained immunophenotypic features of myHSC but lost long-term regenerative potential following expansion (Bernitz et al., 2016, Cell 167, 1296-1309 e10). To distinguish between these two possibilities, equal numbers of myHSC from aged Sema4aKO and WT animals were competitively transplanted into lethally irradiated WT recipients (FIG. 5D). Strikingly, the animals transplanted with aged Sema4aKO myHSC displayed barely detectable levels donor-derived long-term reconstitution (range of donor chimerism 0-1.43%) while robust reconstitution (range of donor chimerism 9.93-77.5%) was observed in the recipients of WT myHSC (FIGS. 5E and 6D). In contrast, recipients of baIHSC from both WT and Sema4aKO mice had comparable levels of donor chimerism (FIGS. 5F and 6E). Taken together, the data demonstrate that during aging, Sema4a absence leads to functional attrition of myHSC but not of baIHSC function.

Aiming to understand the molecular events which are responsible for the myHSC-specific effect of Sema4a, single cell RNA-Seq analysis of myHSC and baIHSC from aged WT and Sema4aKO mice was performed using the Smart-Seq2 protocol (Picelli et al., 2014, Nat Protoc 9, 171-81.). [0145]192 cells per group (768 total) were sorted, of which 642 were selected for analysis following quality control (see Methods). As expected, WT myHSC had a higher expression of Slamf1, self-renewal/low-output-associated genes (CD74, Ly6a, vWF, Procr) and lower expression of cell cycle-related genes (Cdk6 and Mki67) as compared to WT baIHSC (FIG. 6F) (Becker-Herman et al., 2021, PLoS Biol 19, e3001121; Morcos et al., 2017, Stem Cell Reports 8, 1472-1478; Kent et al., 2008, Blood 112, 560-7; Kent et al., 2009, Blood 113, 6342-50; Rodriguez-Fraticelli et al., 2020, Nature 583, 585-589; and Laurenti et al., 2015, Cell Stem Cell 16, 302-13).

A transcriptome-wide analysis revealed that within the myHSC fraction, WT and Sema4aKO cells formed distinct, minimally overlapping clusters (FIG. 5G) while baIHSC of both genotypes merged together, indicating that the absence of Sema4a induces transcriptional changes predominantly in myHSC (FIG. 5H). This HSC subset-specific difference was quantified by detecting a greater correlation-based distance between WT/Sema4aKO myHSC compared to WT/Sema4aKO baIHSC (FIG. 5I); Wilcoxon Rank-Sum test, p-value 3.1e-85, see Methods for further details).

Next, the transcriptional features of the above aged HSC subsets were examined in more detail. Consistent with the results of the clustering analysis, the number of genes which were differentially expressed in aged Sema4aKO vs WT myHSC was much greater (431) compared to aged Sema4aKO vs WT baIHSC (30). In the aged Sema4aKO myHSC signature, markedly reduced expression was observed of genes that normally constrain HSC pool and promote HSC self-renewal (CD74, vWF, Ly6a, Mllt3) (Becker-Herman et al., 2021, PLoS Biol 19, e3001121; Kent et al., 2008, Blood 112, 560-7; Kent et al., 2009, Blood 113, 6342-50; Calvanese et al., 2019, Nature 576, 281-286), consistent with their excessive expansion and loss of stemness (FIG. 6G). Moreover, GSEA demonstrated that a significant enrichment for the terms “p53 pathways” (top genes: Jun, Fos, Sesn1) and “TNF-alpha/NFkB signaling” (top genes: Fosb, Egr1, Jun) and as well as recently defined “core aging HSC signature” (Svendsen et al, 2021, The Lancet 398(10310): p1507-1516) (FIGS. 5J, 5K and 6H).

While “TNF-alpha/NFkB signaling” was also enriched in aged Sema4aKO baIHSC (FIG. 6I), no enrichment for the “p53 pathway” was observed, and enrichment for the “core aging HSC signature” was much weaker (FDR=0.09, P=0.047 for baIHSC vs FDR=0.002, P=0.001 for myHSC, (FIG. 6H and data not shown)). These findings suggest that in the absence of Sema4a, aged myHSC sustain a greater degree of stress- and inflammation-induced damage (Walter et al., 2015, Nature 520, 549-52). Consistent with this notion, in silico cell cycle analysis (Scialdone et al., 2015, Methods 85, 54-61) revealed reduced cycling in aged Sema4aKO myHSC but not in baIHSC (FIG. 5L). While loss of proliferative capacity in myHSC occurs during normal aging (Montecino-Rodriguez et al., 2019, Stem Cell Reports 12, 584-596), it was more prominent in aged Sema4aKO myHSC. Together with other phenotypic (expansion and functional loss) and molecular (aging HSC signature) features of normal aging which were exaggerated in aged Sema4aKO myHSC, this suggests that the absence of Sema4a leads to premature myHSC aging.

Given that amplification of aged Sema4aKO myHSC was accompanied by a marked expansion of downstream myeloid progeny, it was pondered if accelerated differentiation was another factor which would explain their functional loss. This question was addressed using diffusion pseudotime (DPT) analysis (Haghverdi et al., 2016, Nat Methods 13, 845-8), which can quantify the differentiation state of each cell going from naive (corresponding to HSC) to more mature (multipotent progenitors, MPP). To this end, 10× Genomics single cell RNA-Seq profiles of lin-kit⁺ HSPC from WT animals were generated, which were age-matched with WT/Sema4aKO animals for the Smart-Seq2 single-cell RNA-Seq experiment described above. In this 10× dataset, by mapping expression of previously described markers (Nestorowa et al., 2016, Blood 128, e20-31) the clusters that correspond to HSC (Cluster 2; Ly6a, Procr, H/f) and MPP (Cluster 0; Cd34, Cebpa, Ctsg) were identified (see Methods and FIG. 6J-6L). Next, the transcriptomes of cells within these clusters was utilized to estimate a differentiation trajectory (FIG. 6M, see Methods for further details), in which higher DPT values correspond to more mature cells. As expected, analysis of known differentiation marker genes revealed downregulation of vWF, Mpl, Fdg5, Ctnnal1, Procr, and upregulation of Ctsg and Cbpa as cells progressed from HSC to MPP (FIG. 6N).

A DPT value was then estimated along this trajectory for myHSC and baIHSC from aged WT and Sema4aKO mice which were profiled in the Smart-Seq2 experiment described above. Consistent with previously reported myHSC/baIHSC hierarchy, the DPT values of WT aged myHSC were lower than WT baIHSC, indicating that myHSC are more primitive than baIHSC (FIG. 60), (Carrelha et al., 2018, Nature 554, 106-111; and Morita et al., 2010, J Exp Med 207, 1173-82). Importantly, the comparison between aged WT myHSC and aged Sema4aKO myHSC revealed that the DPT values for aged Sema4aKO myHSC were higher, suggesting that they became more differentiated (p-value=0.0002, Wilcoxon rank-sum test (FIG. 5M). Conversely, no significant difference was found between the DPT distributions of aged WT and aged Sema4aKO baIHSC (FIG. 5N). Thus, the DPT analysis demonstrates that the absence of Sema4a during aging leads to premature, myHSC-specific activation of the differentiation transcriptional program.

In sum, the immunophenotypic, functional and transcriptional analysis identified Sema4a as a critical regulator of myHSC self-renewal and differentiation. The profound loss of regenerative capacity in aged Sema4aKO myHSC, as observed in the transplant experiments, likely represents a cumulative effect of partially overlapping cellular defects, such as increased inflammatory damage, premature aging and accelerated differentiation.

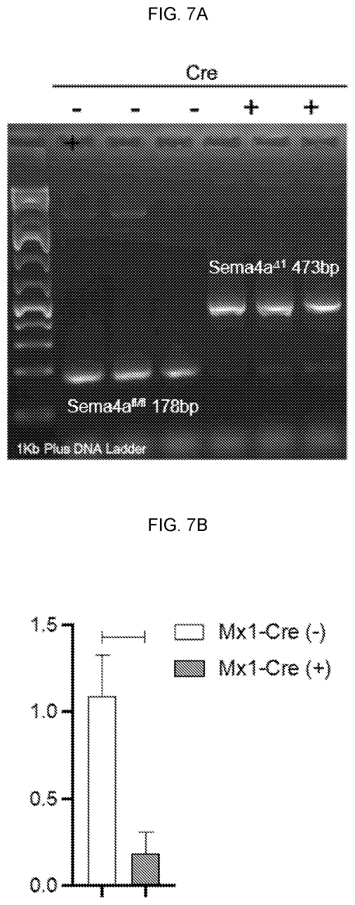

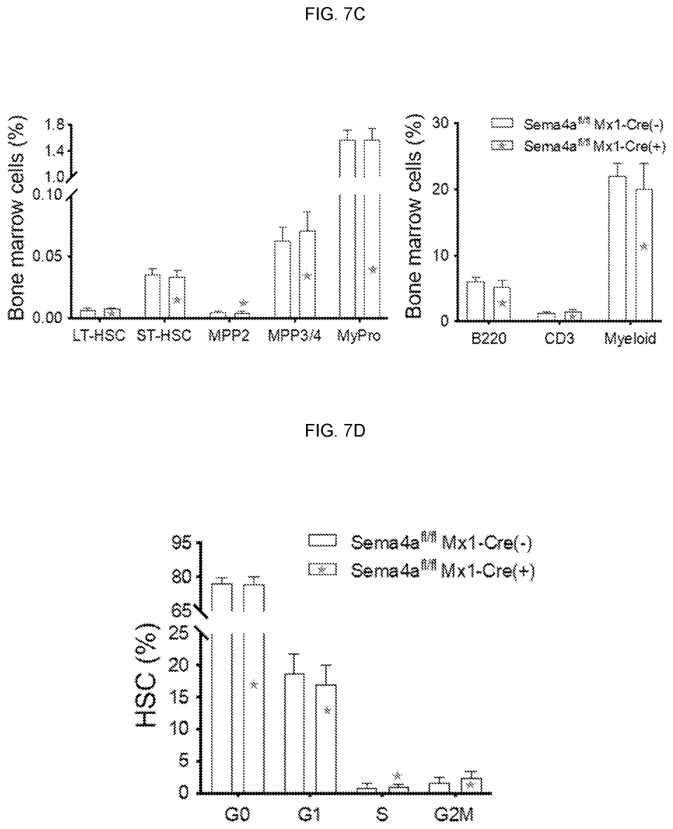

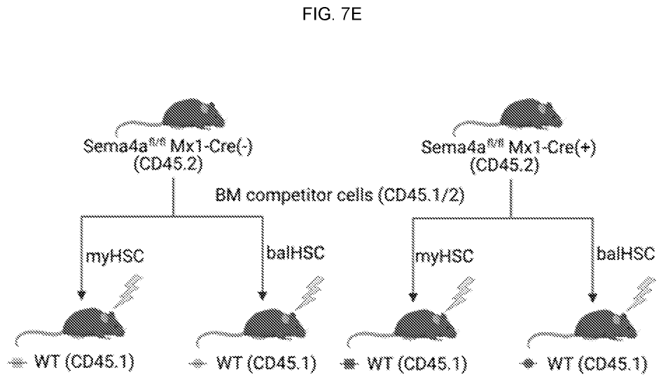

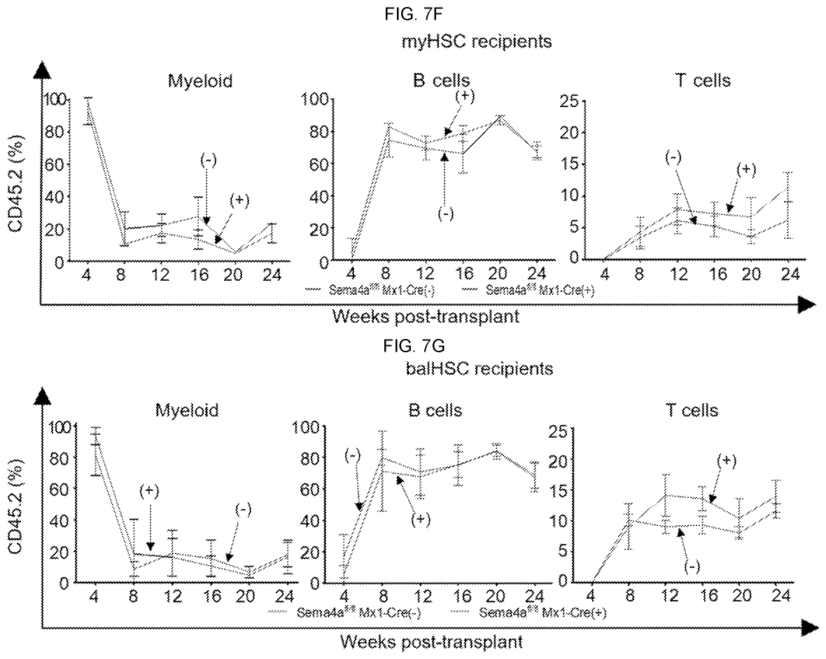

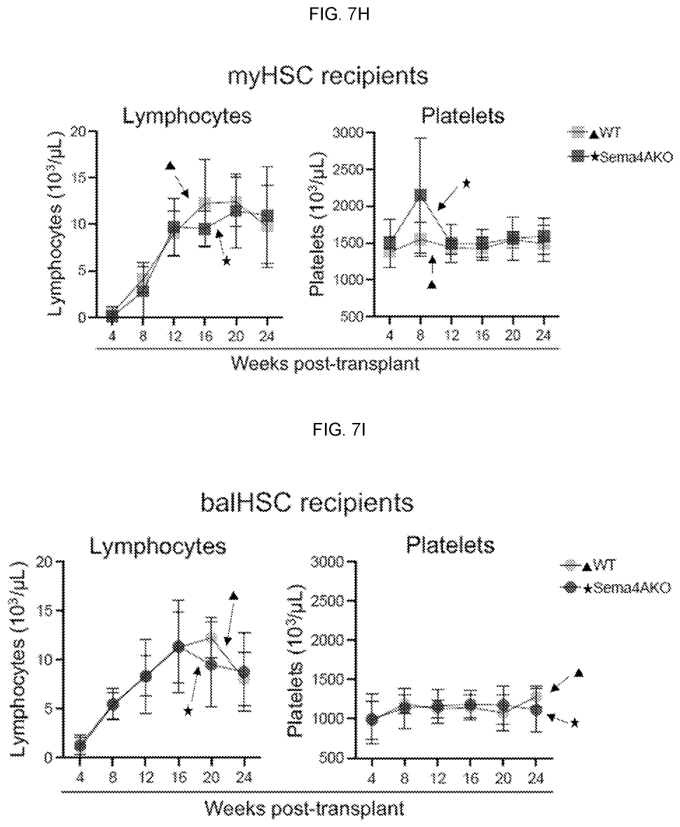

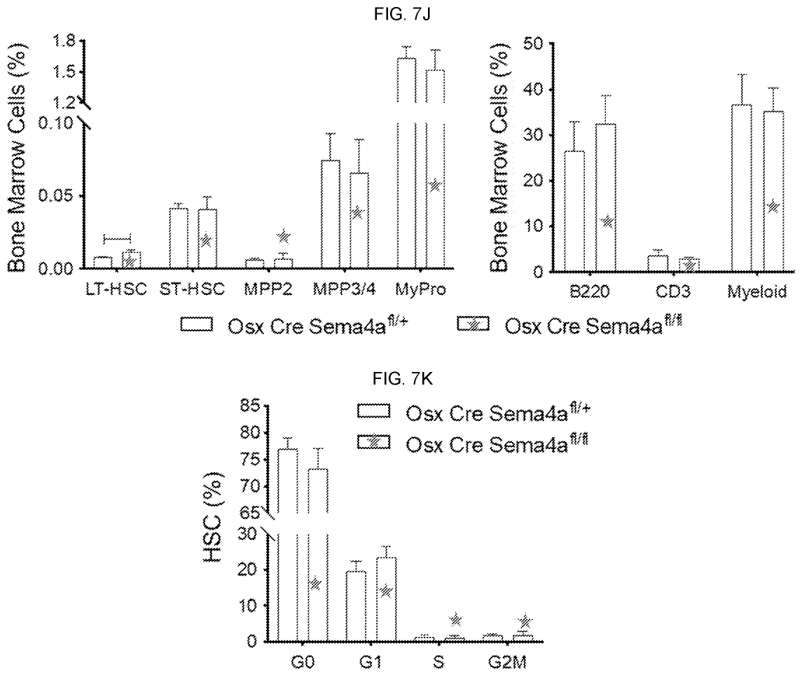

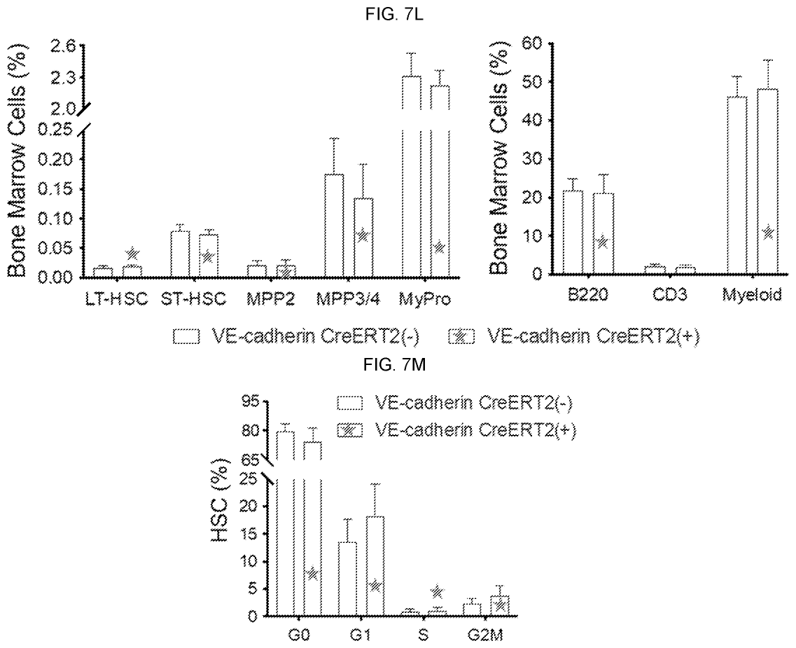

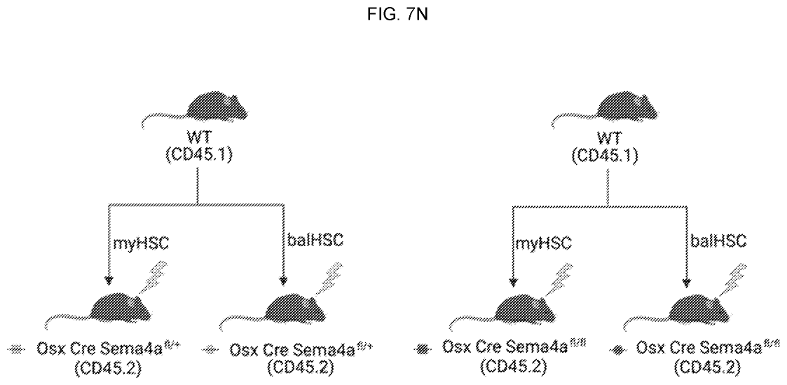

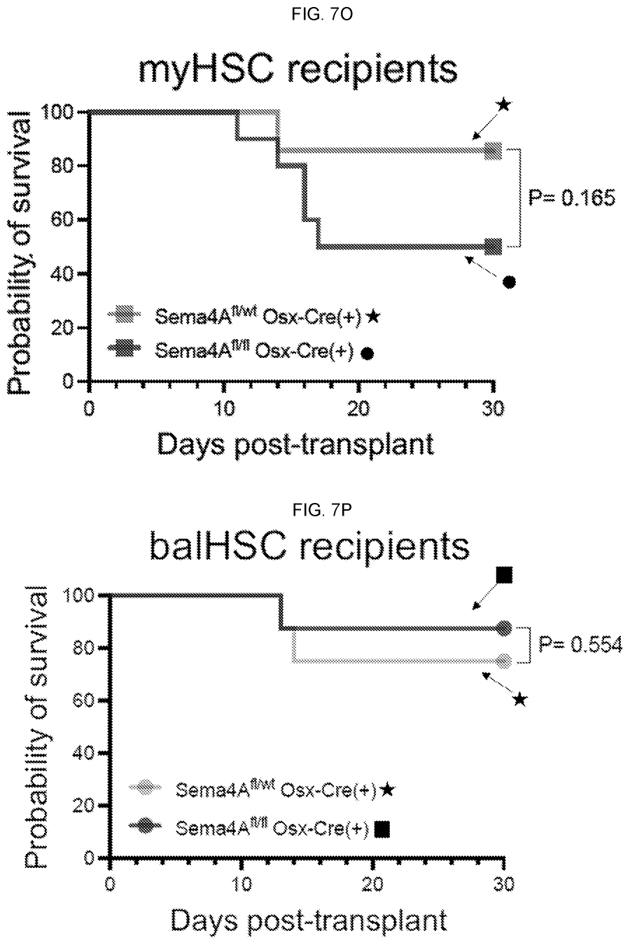

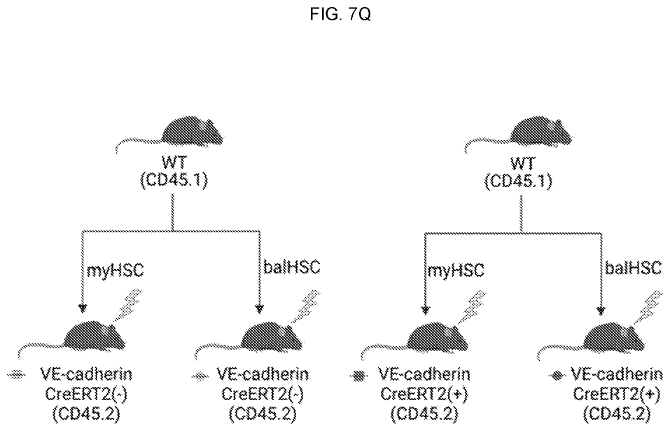

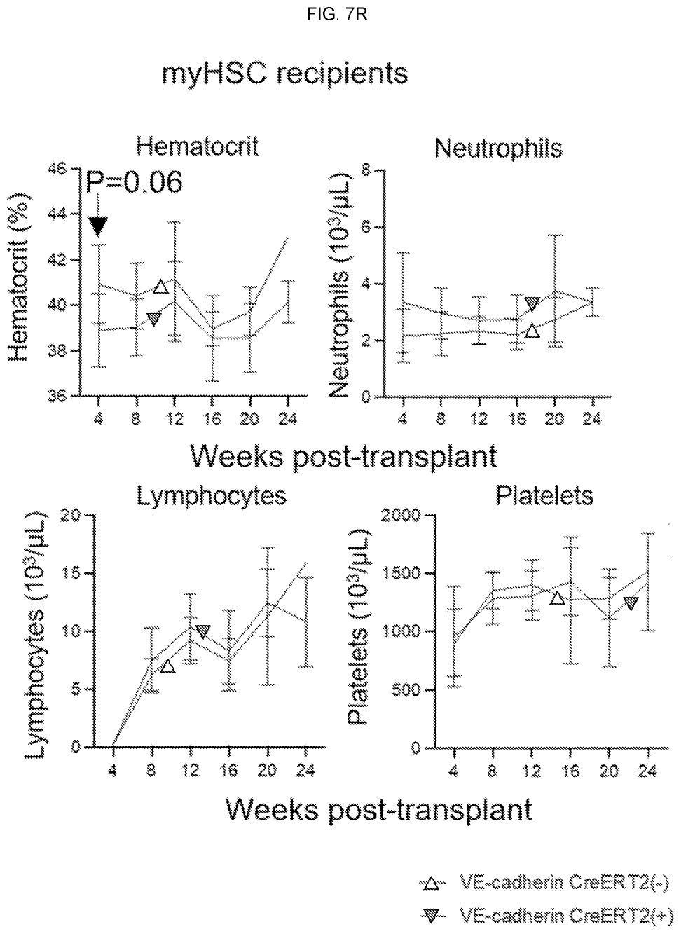

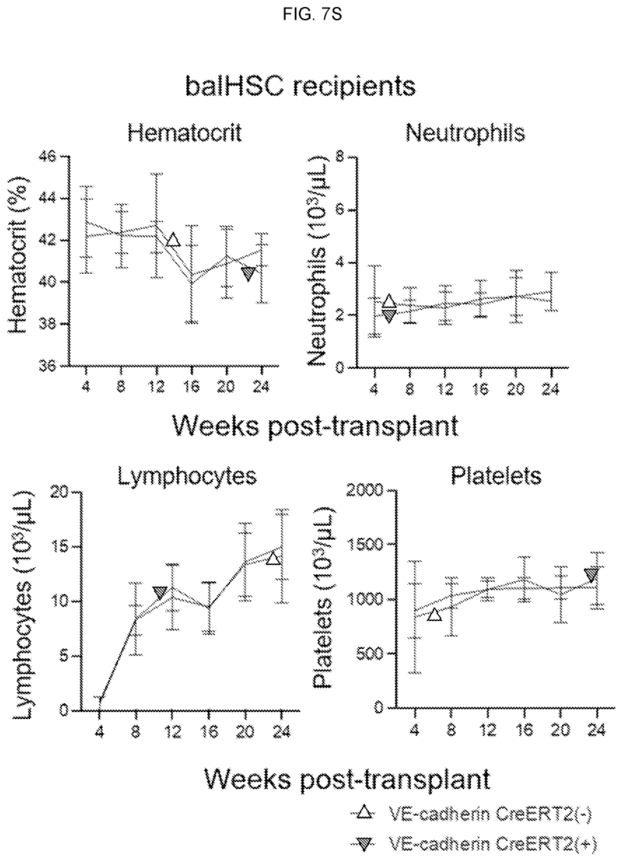

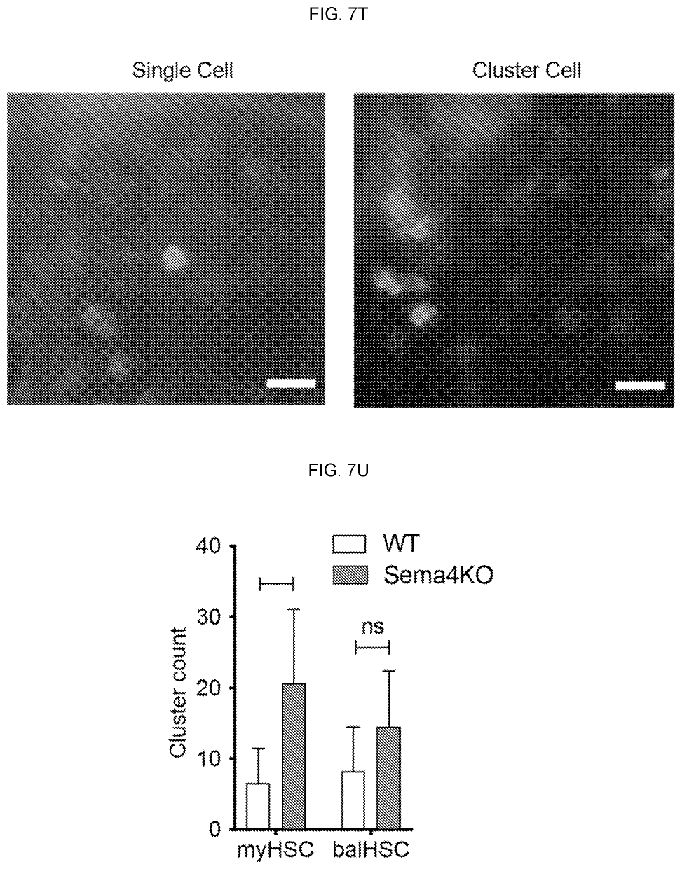

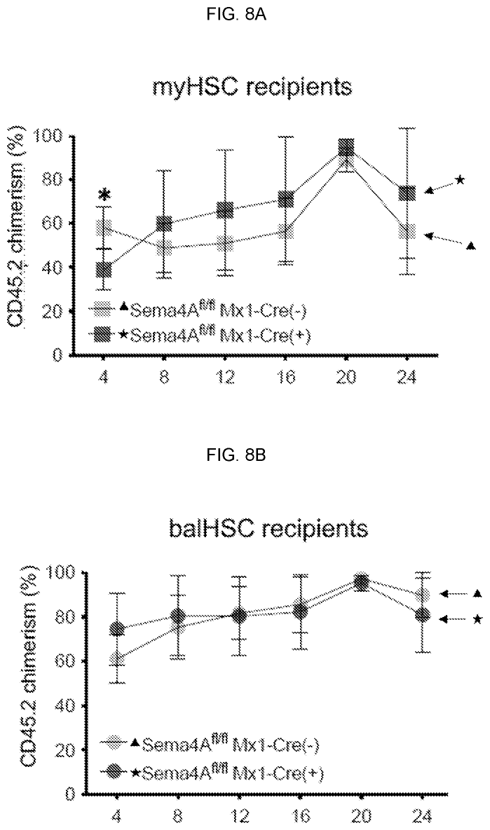

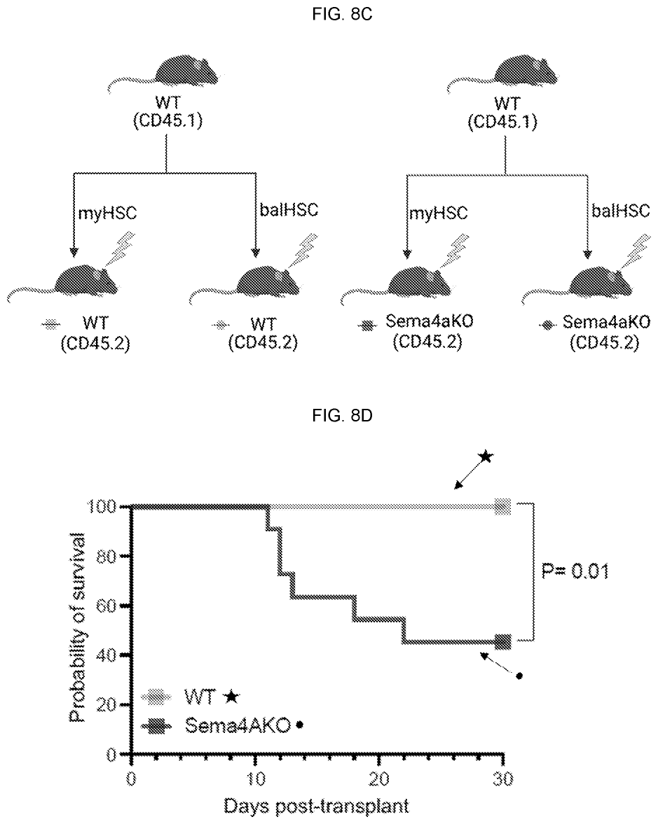

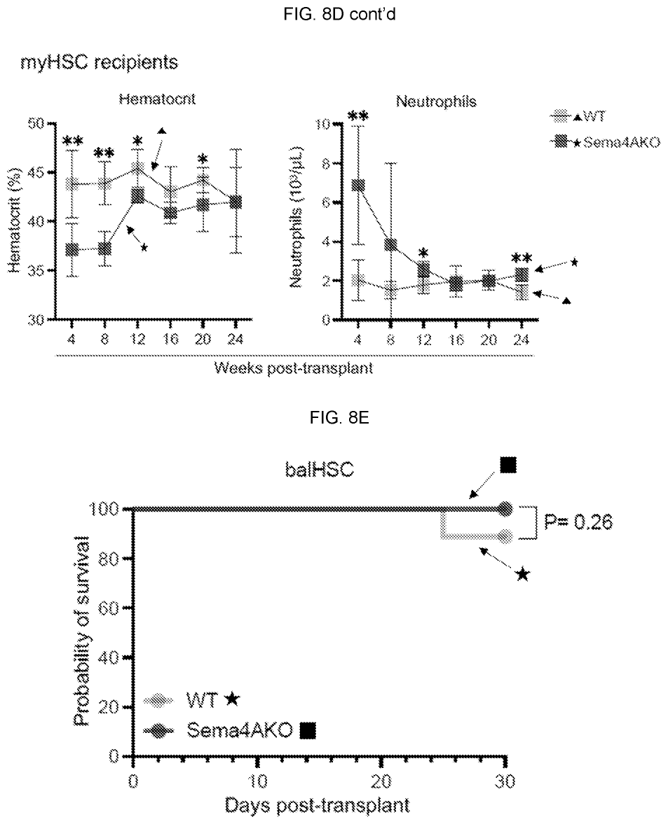

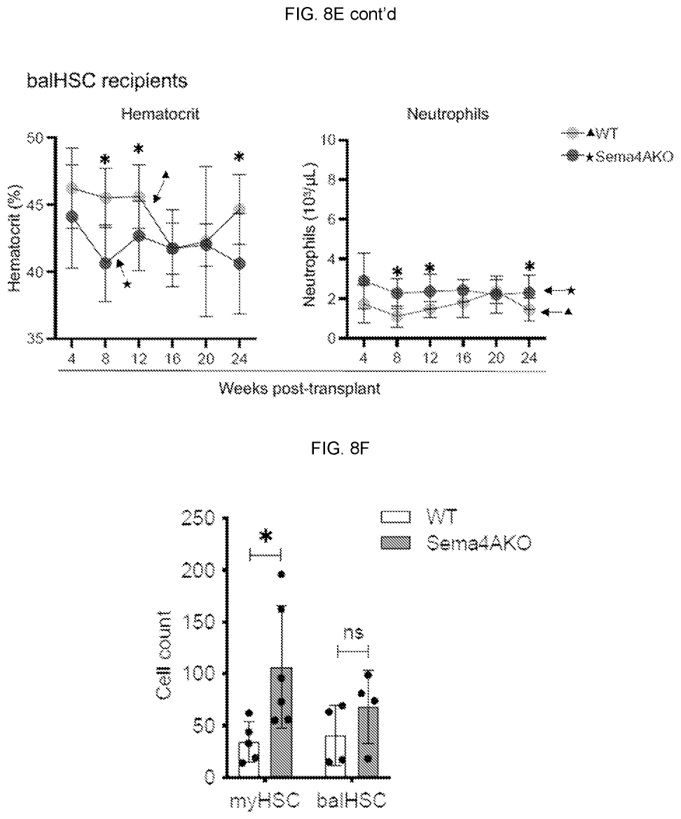

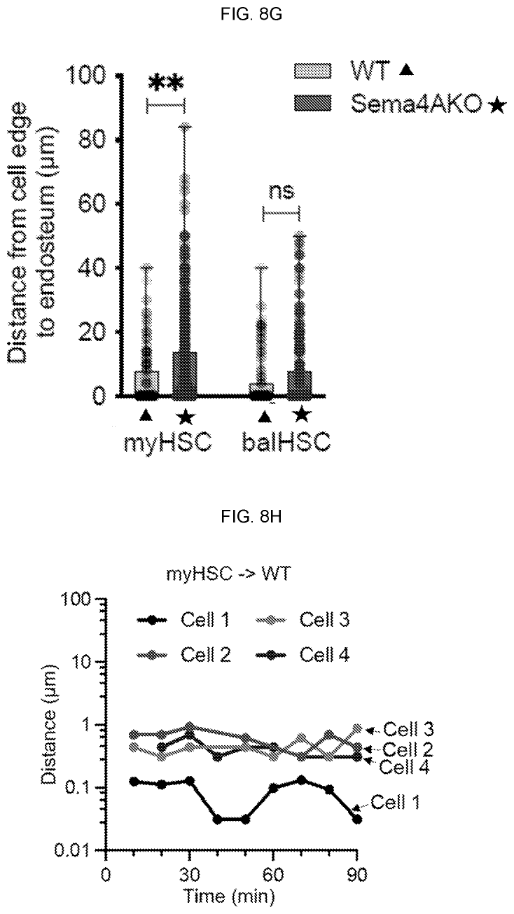

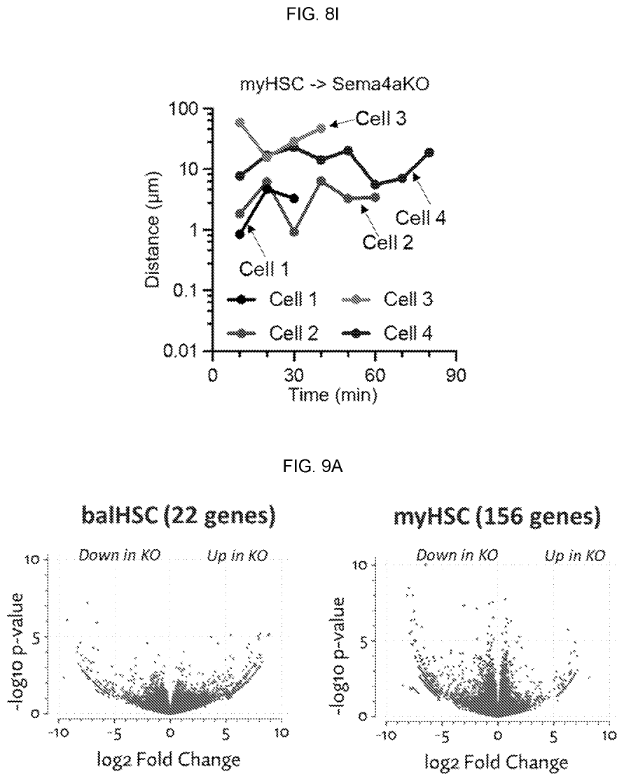

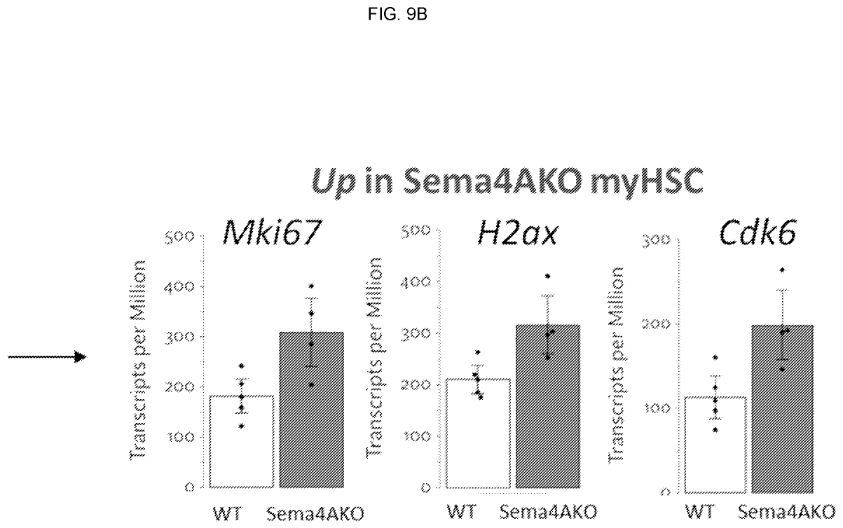

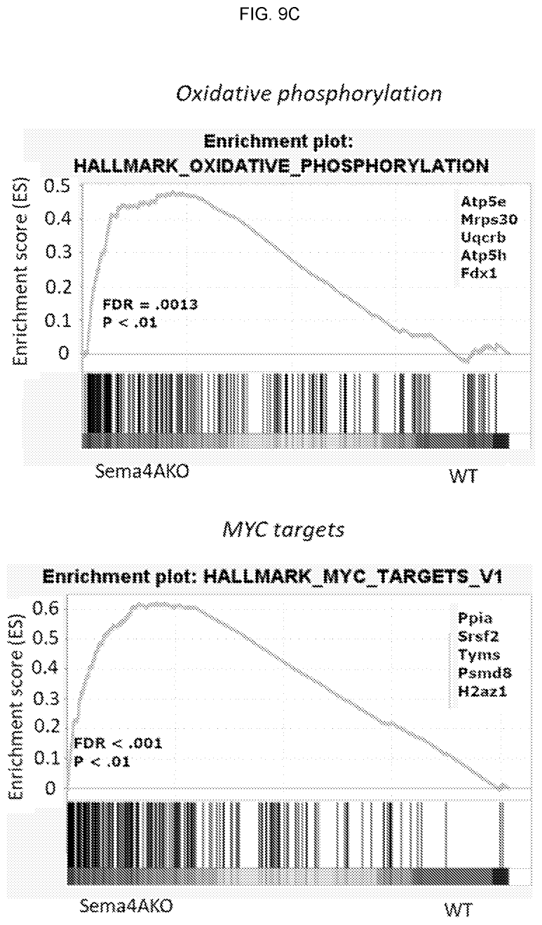

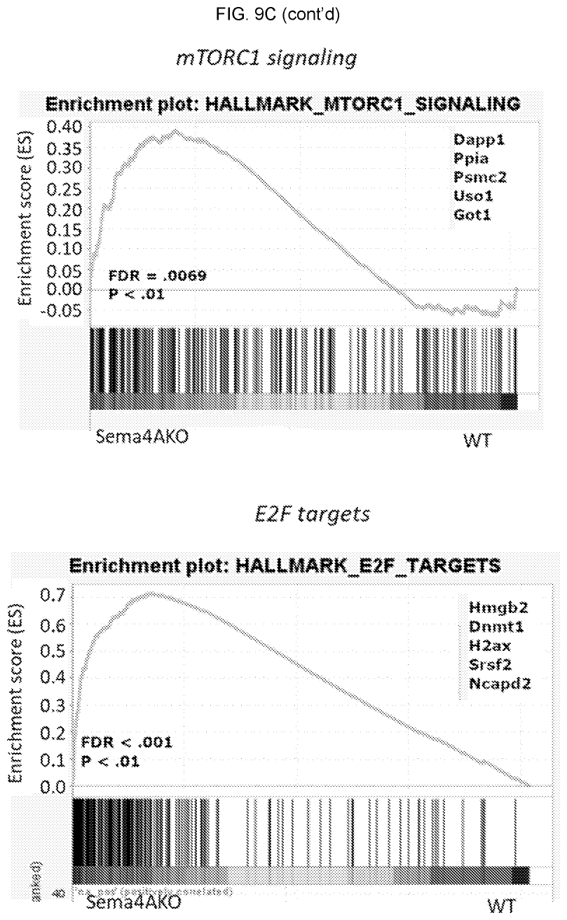

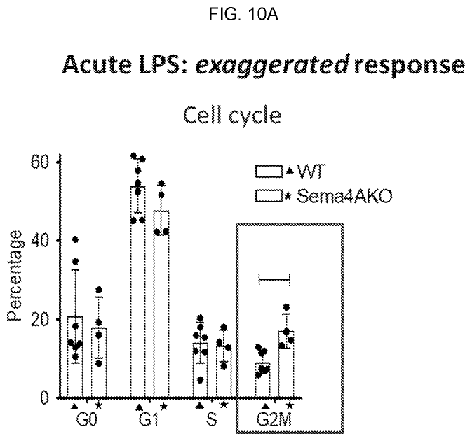

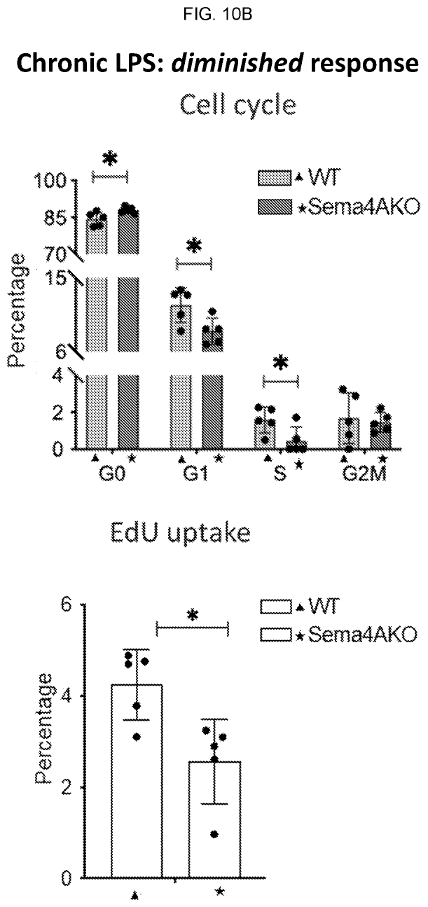

Sema4a from the bone marrow niche controls myHSC proliferation and self-renewal. Since Sema4a is expressed in both non-hematopoietic and hematopoietic cells, including HSCs (Cabezas-Wallscheid et al., 2017, Cell 169, 807-823 e19; Baccin et al., 2020, Nat Cell Biol 22, 38-48; and Cabezas-Wallscheid et al., 2014, Cell Stem Cell 15, 507-522), it was asked which cellular source of Sema4a was functionally indispensable for myHSC function. First, the role of HSC-derived Sema4a was investigated by employing conditional deletion with Mx1-Cre. Cre-induced recombination of “floxed” Sema4a allele was confirmed in HSPC by PCR and Q-PCR analysis (FIGS. 7A and 7B). Analysis of from Sema4a^{fl/fl}Cre⁺ animals at the steady-state revealed no difference in peripheral blood counts, bone marrow cellularity, cell cycle and frequency of HSPC and mature cells, as compared to Sema4a^{fl/fl}Cre⁻ controls (FIGS. 7C and 7D). In competitive transplantation assay, no difference in long-reconstitution capacity of myHSC and balHSC from Sema4a^{fl/fl}Cre⁺ and Sema4a^{fl/fl}Cre mice was observed (FIGS. 8A-8B and 7E-7G). Taken together, these data indicate that hematopoietic-derived Sema4a is dispensable for myHSC and baIHSC function.

In order to elucidate the role of microenvironment-derived Sema4a, lethally irradiated WT and Sema4aKO recipients were non-competitively transplanted with a radioprotective dose of myHSC and baIHSC from WT mice (FIG. 8C). Strikingly, a 50% post-transplant mortality was observed in Sema4aKO recipients of myHSC (FIG. 8D). These animals also developed marked anemia and neutrophilia thus resembling blood count changes in aged Sema4aKO mice (FIG. 8D and FIG. 7H). In contrast, no survival difference was observed in the recipients of baIHSC, which displayed only mild alterations in blood counts (FIG. 8E and FIG. 7I). This experiment revealed that Sema4a from the host hematopoietic niche is critical for myHSC self-renewal under stress but plays no significant role in baIHSC regeneration.

Among the subsets which make up the bone marrow niche, Sema4a is expressed by endothelial and osteoprogenitor cells (FIGS. 2A and 2B). In order to refine their physiological relevance as cellular sources of Sema4a in the niche, Sema4a was conditionally deleted from each of the two cell types by crossing “floxed” Sema4aKO mice to either VECad-CreER^{T2} or Osx-Cre animals. Steady-state analysis of Sema4a^{fl/fl}VECad-CreER^{T2+} and Sema4a^{fl/fl} Osx-Cre⁺ mice revealed no difference in peripheral blood counts, bone marrow cellularity, cell cycle and frequency of HSPC and mature cells, as compared to Sema4a^{fl/fl}CreER^{T2-} (Sorensen et al., 2009, Blood 113, 5680-8) and Sema4a^{fl/wt}Osx-Cre⁺ (Rodda and McMahon, 2006, Development 133, 3231-44) controls, respectively (FIGS. 7J-7M). Transplantation of WT myHSC and balHSC into lethally irradiated donors of the above genotypes showed that both endothelial- and osteoprogenitor-specific deletion of Sema4a only partially recapitulated the effect of a complete Sema4a absence in the host. Specifically, while increased mortality was observed in Sema4a^{fl/fl}Osx-Cre⁺ recipients of myHSC, the difference was not statistically significant (FIGS. 7N-7P), whereas all Sema4a^{fl/fl}CreER^{T2+} survived and developed only mild anemia (FIG. 7Q-7S). Taken together, these studies show that a cumulative production by osteoprogenitors, endothelial cells and likely other cellular source(s) may be responsible for the full functional effect of niche-derived Sema4a on myHSC.

Having demonstrated that a complete absence of Sema4a in the host is critical for myHSC engraftment (FIG. 8D), it was asked how it affects early homing, expansion and motility of transplanted myHSC in real time. To this end, myHSC and baIHSC were isolated from WT mice and fluorescently labeled with DiD (1,1-Dioctadecyl-3,3,3,3-tetramethylindodicarbocyanine). Then, equal numbers of these cells were transferred into lethally irradiated WT and Sema4aKO recipients and intravital time-lapse two-photon microscopy of the calvarial bone marrow was performed (Christodoulou et al., 2020, Nature 578, 278-283). 3D z-stacks and time-lapse movies were recorded 12-15 hours after the cell injection. Single cells and clusters (defined as two or more cells) were detected throughout the calvarial bone marrow in all mice (FIG. 7T). Notably, a 3 times higher number of transplanted cells was observed in Sema4aKO recipients of myHSC compared to WT controls (mean 10⁶ cells vs. 34 cells, respectively [p-value=0.0295]) whereas cell number in WT/Sema4a recipients of baIHSC was not significantly different (mean 68 cells vs. 41 cells, respectively [p-value=0.2793]) (FIG. 8F). These results indicate that the absence of Sema4a in the niche leads to excessive myHSC expansion but is inconsequential for baIHSC. As further evidence for this, a similar 3-fold increase in the number of cell clusters was found in the Sema4aKO recipients of myHSC as compared to WT controls (mean 21 vs. 7 clusters [p-value=0.0219] whereas the trend in the baIHSC recipients was much weaker (15 vs. 8 clusters [p-value =0.2591]) (FIG. 7U).

Next, the effect of Sema4a on myHSC and baIHSC localization was analyzed by measuring the 3D distance to the endosteal surface, an established location of post-transplant HSC niche (Lo Celso et al., 2009, Nature 457, 92-6). Importantly, it was observed that in Sema4aKO recipients of myHSC, transplanted cells were found nearly 2× farther from the endosteum compared to WT controls (mean 8.2 μm vs. 4.9 μm, respectively [p-value=0.0024]) whereas this difference was smaller and not statistically significant in the Sema4aKO/WT baIHSC recipients (mean 5.9 μm vs. 4.0 μm, respectively [p-value=0.0674]) (FIG. 8G). These results suggest that in the absence of host Sema4a, myHSC homed away from the niche, whereas localization of baIHSC was only marginally altered.

Recent intra-vital time-lapsed microscopy studies revealed that upon proliferative challenge, some HSC within the bone marrow niche become motile (Christodoulou et al., 2020, Nature 578, 278-283; and Upadhaya et al., 2020, Cell Stem Cell 27, 336-345 e4), indicating that motility may reflect HSC activation state. In order to investigate if motility of transplanted myHSC and baIHSC is altered in the absence of Sema4a, time-lapse microscopy was performed over the 1.5-hour imaging session. It was found that baIHSC displayed limited motility (defined as <5 μm movement of the cell centroid over the imaging period) regardless of the host genotype (data not shown). In contrast, a small fraction (4.7% of total) of myHSC transplanted into Sema4aKO mice exhibited highly motile behavior, which was consistent with the lack of inhibitory effect of Sema4a (FIGS. 8H and 8I). In sum, the intravital imaging data suggest that host absence of Sema4a leads to myHSC hyperactivation, excessive proliferation and mis-localization, which cumulatively may contribute to the loss of self-renewal and engraftment failure in Sema4aKO recipients of myHSC. Intriguingly, post-transplant behavior of baIHSC was relatively unaffected, indicating that the two HSC subsets may have fundamentally different requirements for engraftment, including specific dependence of myHSC on Sema4a.

Discussion Studies described herein provide substantial experimental support for the concept that functionally diverse subsets of somatic stem cells are controlled by distinct non cell-autonomous signals. Prior studies have indicated that within the HSC pool, myHSC and baIHSC display differential sensitivity to soluble factors, such as TGF-beta, RANTES, CXCL2 and histamine (Challen et al., 2010, Cell Stem Cell 6, 265-78; Ergen et al., 2012, Blood 119, 2500-9; Pinho et al., 2018, Dev Cell 44, 634-641 e4; and Chen et al., 2017, Cell Stem Cell 21, 747-760 e7). However, the impact of these molecules on myHSC longevity and interaction with the bone marrow microenvironment has not been investigated in detail.

In the current example, Sema4a is identified as a potent and specific regulator of myHSC quiescence and self-renewal. Semaphorins and plexins are large protein families (Alto and Terman, 2017, Methods Mol Biol 1493, 1-25) whose role in regulation of adult stem cell quiescence and self-renewal is not known. The absence of Sema4a is demonstrated to lead to myHSC over-proliferation and hyperactivation following acute inflammatory insult, which correlates with a dramatic loss of regenerative function with age, likely due to the unopposed effects of inflammatory signaling over animal's lifetime (Kaschutnig et al., 2015, Cell Cycle 14, 2734-42). Notably, in a wild-type situation, myHSC are preferentially activated by inflammation (Mann et al., 2018, Cell Rep 25, 2992-3005 e5; Matatall et al., 2014, Stem Cells 32, 3023-30; and Mitroulis et al., 2018, Cell 172, 147-161 e12) and become vulnerable to damage, underscoring a physiological need for a dedicated protective signal, such as Sema4a.

Excessive myeloid expansion, as observed in the aged Sema4aKO model, is the cardinal feature of human hematopoietic aging (Pang et al., 2011, Proc Natl Acad Sci USA 108, 20012-7) and clonal hematopoiesis of indetermined significance (CHIP)—a common condition which carries a significant risk of progression to myeloid malignancy over time but lacks effective therapeutic intervention (Jaiswal and Ebert, 2019, Science 366).

Findings disclosed herein indicate that pharmacological augmentation of Sema4a/PlxnD1 signaling can serve as a strategy to constrain proliferation of myHSC at the top of expanding myeloid-biased clones, thereby preventing aging-associated HSC dysfunction and reducing the risk of malignant transformation. In addition, they suggest that aberrant Sema4a/PlxnD1 signaling can contribute to the pathogenesis of myeloid malignancies and chronic inflammatory disorders.

The disclosed results underscore the importance of niche-derived signals in life-long maintenance of tissue stem cell hierarchy, which is topped by myHSC in the hematopoietic system. Given that stem cell hierarchies underlie functional organization of other tissues (Scaramozza et al., 2019, Cell Stem Cell 24, 944-957 e5; Farrelly et al., 2021, Cell Stem Cell 28, 1233-1247 e4; Altshuler et al., 2021, Cell Stem Cell 28, 1248-1261 e8; Rompolas et al., 2013, Nature 502, 513-8; and Hsu et al., 2011, Cell 144, 92-105), the data presented here provide justification for broader efforts to identify subset-specific stem cell regulators, which may lead to development of more precise and effective pro-regenerative therapies.

Bioinformatics Methods. Raw data processing and normalization. The abundance of the transcripts from 768 cells were quantified with salmon (v0.17). First, the mouse transcriptome (GRCm38) was indexed in quasi-mapping-based mode with —seqBias and —gcBias flags. Then, the transcript level abundances were aggregated into gene level abundances, which in turn were transformed into a gene-cell count matrix. Next quality control was performed to filter out cells that satisfy any of the following criteria: 1) less than 4000 genes detected (detection threshold: reads-per-million>10), 2) overall mapping less than 50%, 3) fraction of reads mapping to mitochondrial transcripts larger than 0.2, 4) fraction of reads mapping to ERCC spike-ins higher than 0.1. After quality control, 642 cells were retained for downstream analyses (155 WT myHSC, 157 WT baIHSC, 166 KO myHSC, 164 KO baIHSC). The data were normalized using ‘quickcluster’ and ‘computeSumFactors’ functions from the scran package in R (Lun et al. 2016). Finally, a pseudocount of 1 was added to the count matrix, followed by natural-log transformation.

Batch integration and visualization. Since the data were collected from two separate batches, batch integration was performed before visualizing the data. For this purpose, the Seurat batch integration workflow was used. First, 3000 highly variable genes were selected from each batch. Then, the FindIntegrationAnchors function was used to estimate the anchors to use for the integration with 3000 features (anchor.features=3000) and the first 20 canonical variates (dims =1:20). Finally, the ‘IntegrateData’ function was used with default parameters to integrate the two batches.

To visualize the cells on a low dimensional space, a k-nearest neighbor graph was first built with ‘neighbors’ function from scanpy with 10 principal components (PCs) and k=30. Then, the ‘tl.umap’ function was used to calculate a UMAP representation and the first two UMAP components were plotted. This procedure was applied separately for myHSCs and balHSCs to visualize how these cells are differentially affected by the absence of Sema4a.

To verify if myHSCs and balHSCs are differentially affected by the absence of Sema4a, pairwise Spearman's correlation distance (defined as \sqrt{(1-ρ)/2}, where ρ is the Spearman's correlation coefficient between the cells) was calculated between WT and KO cells for myHSCs and balHSCs separately. Then, the statistical significance of the difference was tested between the two distributions of pairwise distances by using the Wilcoxon rank-sum test.

Cell cycle prediction. The cell cycle phase of each cell was estimated by applying the “pairs” algorithm, described by Scialdone et al. (2015, Methods 85, 54-61). The implementation of the algorithm on R (scran package) was used in this analysis. More specifically, ‘cyclone’ was used with a minimum number of pairs 120 (mm.pairs=120). To test the difference in percentage of cells allocated to G2/M phase between WT and KO myHSC, the number of cells allocated to G1 and S phases were merged for each condition. Finally, a contingency table was built and Pearson's chi-square test was performed with ‘chi2_contingency’ function from the ‘scipy’ module in python.

Diffusion pseudotime analysis. For this analysis, 10× data generated from WT aged mice was considered as the reference. Specifically, HSCs and multipotent progenitors (MPPs) were chosen. The myHSC cells (WT and KO) were integrated with these cells with the Seurat integration pipeline as described elsewhere herein. Next, a diffusion map was used to establish a trajectory. For this purpose, a k-nearest neighbor graph was first built with the first 5 PCs and k=15. The diffusion map was constructed with the ‘tl.diffmap’ function from scanpy. A diffusion pseudotime (dpt) was defined by selecting the root cell that had the lowest value of the first diffusion component (DC1), since the HSC differentiation towards MPPs was assumed to be along the DC1. Finally, in order to test the difference in the distribution of dpt values for both WT and KO myHSCs, the Wilcoxon rank-sum test was used. The procedure for the balHSCs was performed the same way.

Differential gene expression analysis. In order to find genes that were differentially expressed between KO and WT myHSC, the DEseq2 package was used on R. First, genes were removed that were expressed in less than 10 cells for each batch. Then the counts were rounded to integer. A DESeq object was created from the count matrix with the design condition+batch. Fold-changes and p-values were estimated using the DESeq function with default parameters. These results were then visualized with EnhancedVolcano package on R.

10× single cell analysis. Libraries from Kit+ cells from WT (n=1) and Sema4a KO (n=2) young and old mice were prepared using the Single Cell 3′ Reagent Kit v3 (10× Genomics). The libraries were prepared according to the manufacturer's protocol.

The Cell Ranger 3.1.0 (10× Genomics) analysis pipeline was used to process the 10× single cell RNA-Seq output by aligning reads to mm 10-3.0.0 mouse transcriptome (Ensembl). SCANPY was used for the following steps 1-3.
1) Filter out cells with less than 200 genes.
2) Filter out genes that were in less than 3 cells.
3) Filter out cells with a) high percentage mitochondrial genes (>5) and b) a high number of genes (>6500).
4) The tool Scrublet (Wolock et al., 2019) was used to predict and remove doublets from each dataset. The WT and Sema4aKO datasets were concatenated and SCANPY was used for the following steps 5-15.
5) Normalize reads per cell.
6) Logarithmize the data.
7) Regress out the effects of total counts per cell and the percentage mitochondrial genes.
8) Identify highly variable genes for principal component analysis (PCA)
9) Compute PCA
10) Compute the neighborhood graph (n_neighbors=10).
11) Embed the neighborhood graph in 2 dimensions using Uniform Manifold Approximation and Projection (UMAP) (McInnes, Healy and Melville, 2018, http://arxiv.org/abs/1802.03426).
12) Cluster the neighborhood graph using Leiden (Traag et al., 2019, Scientific Reports, 9(5233)).
13) Find marker genes per cluster using a Wilcoxon rank-sum (Mann-Whitney-U) test. Differentially expressed genes between WT and Sema4aKO cells were found within in each cluster.
14) Map gene expression onto the UMAP embedding.
15) Generate additional graphical plots. Partition-based graph abstraction (PAGA) (Wolf et al., 2019, Genome Biology, 20(59) was used to make lineage inferences from the neighborhood graph, using the Leiden clustering.

Pathway analysis of differentially expressed genes between WT and Sema4aKO for each cluster were ranked by significance and pathway enrichment analysis was performed using the GSEA tool from the broad institute (Subramanian et al., 2005, PNAS, 102(43): 15545-15550). In particular annotated gene sets from the following databases were used; Hallmark gene sets (Liberzon et al., 2015, Cell Syst., 1(6):417-425), BioCarta, KEGG (Kanehisa and Goto, 2000, Nucleic Acids Res., 28(1):27-30), Reactome (Croft et al., 2011, Nucleic Acids Res., 39 (Database issue):D691-7) and Gene ontology (GO) (Ashburner et al., 2000; and Gene Ontology Consortium, 2019, Nucleic acids research 47(D1), D330-D338). Genes were input as a ranked list by Wald statistic and pathways were considered significant with a false discovery rate (FDR) of <0.1.

(x) Closing Paragraphs. Section headings are provided for organizational purposes only and do not limit the scope or interpretation of the disclosure.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to modulate the myeloid arm of the immune system, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of protecting myeloid-biased hematopoietic stem cells (myHSC) from a proliferative challenge, the method comprising administering a therapeutically effective amount of a PlexinD1 receptor agonist or a Sema4a agonist to the myHSC.

2. The method of claim 1, comprising administering the therapeutically effective amount of the PlexinD1 receptor agonist to the myHSC.

3. The method of claim 2, wherein the PlexinD1 receptor agonist comprises Sema4a.

4. The method of claim 3, wherein the Sema4a is recombinantly-produced.

5. The method of claim 1, wherein the proliferative challenge comprises acute inflammatory stress, injury, aging, transplant, chemotherapy, or irradiation.

6. The method of claim 5, wherein the infection comprises a bacterial infection, a viral infection, a fungal infection, or a yeast infection.

7. The method of claim 1, wherein the protecting is evidenced by reduced entry of myHSC into the cell cycle as compared to myHSC exposed to the same proliferative challenge in the absence of the therapeutically effective amount of the PlexinD1 receptor agonist or the Sema4a agonist.

8. The method of claim 1, wherein the protecting is evidenced by reduced differentiation of the myHSC as compared to myHSC exposed to the same proliferative challenge in the absence of the therapeutically effective amount of the PlexinD1 receptor agonist or the Sema4a agonist.

9. A method for maintaining myeloid-biased hematopoietic stem cells (myHSC) in a subject in need thereof comprising administering a therapeutically effective amount of a composition that signals the Sema4a/PlexinD1 receptor signaling pathway in the subject, thereby maintaining the myHSC in the subject in need thereof.

10. The method of claim 9, wherein the composition comprises Sema4a.

11. The method of claim 10, wherein the Sema4a is recombinantly-produced.

12. The method of claim 9, wherein the subject is in need thereof due to the presence of a proliferative challenge to the myHSC.

13. The method of claim 9, wherein the proliferative challenge to the myHSC comprises acute inflammatory stress, injury, aging, transplant, infection, chemotherapy, or irradiation.

14. The method of claim 13, wherein the infection comprises a bacterial infection, a viral infection, a fungal infection, or a yeast infection.

15. The method of claim 9, wherein the maintained myHSC are evidenced by reduced myHSC entry of the myHSC into the cell cycle as compared to myHSC in a comparable subject in need thereof that was not administered the composition.

16. The method of claim 9, wherein the maintained myHSC are evidenced by reduced differentiation of the myHSC as compared to myHSC in a comparable subject in need thereof that was not administered the composition.

17. The method of claim 16, wherein the comparable subject in need thereof has a common condition with the subject in need thereof.

18. The method of claim 17, wherein the common condition is acute inflammatory stress, injury, transplant, infection, chemotherapy, or irradiation.

19. The method of claim 18, wherein the infection comprises a bacterial infection, a viral infection, a fungal infection, or a yeast infection

20. The method of claim 16, wherein the comparable subject in need thereof is within an age range of the subject in need thereof.

21. The method of claim 20, wherein the age range is within 2 years.

22. A method for modulating the balance of immune cells in the bone marrow of a subject, the method comprising stimulating Sema4a and/or a PlexinD1 receptor in the subject.

23. The method of claim 22, wherein the stimulating is through administering a PlexinD1 receptor agonist.

24. The method of claim 23, wherein PlexinD1 receptor agonist comprises Sema4a.

25. The method of claim 24, wherein the Sema4a is recombinantly-produced.

26. The method of claim 22, wherein the stimulating increases the percentage of lymphoid cells and decreases the percentage of myeloid cells in the bone marrow as compared to before the stimulating.

27. A method of modulating the activation state of the myeloid arm of immune system comprising stimulating or inhibiting Sema4a and/or a PlexinD1 receptor.

28. The method of claim 27, wherein the modulating down-regulates or dampens the activation state of the myeloid arm of the immune system by stimulating Sema4a and/or a PlexinD1 receptor.

29. The method of claim 28, wherein the stimulating is through administering a PlexinD1 receptor agonist.

30. The method of claim 29, wherein PlexinD1 receptor agonist comprises Sema4a.

31. The method of claim 30, wherein the Sema4a is recombinantly-produced.

32. The method of claim 28, wherein the down-regulating and/or dampening reduces myHSC proliferation and differentiation, as compared to myHSC in the absence of the stimulating.

33. The method of claim 32, wherein the down-regulating and/or dampening reduces myHSC proliferation and differentiation during ex vivo cell manufacturing.

34. The method of claim 27, wherein the modulating dampens a response to a proliferative challenge.

35. The method of claim 34, wherein the proliferative challenge comprises acute inflammatory stress, injury, aging, transplant, infection, chemotherapy, or irradiation.

36. The method of claim 35, wherein the infection comprises a bacterial infection, a viral infection, a fungal infection, or a yeast infection.

37. The method of claim 27, wherein the modulating up-regulates or heightens the activation state of the immune system by inhibiting Sema4a and/or a PlexinD1 receptor.

38. The method of claim 37, wherein the heightening increases the response of myHSC to a physiological challenge.

39. The method of claim 38, wherein the physiological challenge comprises cancer, an infection, or a vaccine.

40. The method of claim 39, wherein the cancer is leukemia.

41. The method of claim 40, wherein the leukemia is acute myeloid leukemia (AML).

42. The method of claim 39, wherein the infection comprises a bacterial infection, a viral infection, a fungal infection, or a yeast infection.

43. The method of claim 37, wherein the up-regulating and/or heightening results in increased entry myHSC into the cell cycle as compared to myHSC not exposed to the inhibiting.

44. The method of claim 37, wherein the up-regulating and/or heightening results in increased differentiation of myHSC as compared to myHSC not exposed to the inhibiting.

45. A composition comprising a therapeutically effective amount of Sema4a and a pharmaceutically acceptable carrier.

46. The composition of claim 45, wherein the Sema4a is recombinantly-produced.
